(19) United States
(12) Patent Application Publication
(10) Pub. No.: US 2004/0161817 A1
(43) Pub. Date: Aug. 19, 2004

(54) Compositions and methods for high-level, large-scale production of recombinant proteins

(75) Inventors: Trish Benton, Moss Beach, CA (US); Christopher Robert Bebbington, San Mateo, CA (US); Karla Ann Henning, Burlingame, CA (US); David J. King, Belmont, CA (US); Robert Crombie, Barbridge (GB); Xiang Shao, Santa Clara, CA (US)

Correspondence Address: COZEN O'CONNOR, P.C., 1900 MARKET STREET, PHILADELPHIA, PA, 19103, US

(73) Assignee: Corixa Corporation, Seattle, WA (US)

(21) Appl. No.: 10/163,863

(22) Filed: Jun. 4, 2002

Related U.S. Application Data
(60) Provisional application No. 60/352,404, filed on Jan. 29, 2002.
(60) Provisional application No. 60/333,620, filed on Nov. 26, 2001.
(60) Provisional application No. 60/295,961, filed on Jun. 4, 2001.

(51) Int. Cl. C12P 21/02; C12N 5/06; C12N 15/85

(52) U.S. Cl. 435/69.1; 435/455; 435/325

(57) ABSTRACT

Compositions and methods for the high-level, large-scale production of recombinant proteins are disclosed. Illustrative compositions comprise one or more expression vectors capable of high-level protein and/or polypeptide expression in combination with an immortalized host cell-line capable of growth in serum-free, suspension culture. Bi-directional UCOE vectors that permit the simultaneous, high-level expression of two or more recombinant proteins and/or polypeptides from a single UCOE based plasmid vector.

### CROSS REFERENCE TO RELATED APPLICATIONS

This application is related to U.S. Provisional Application No. 60/352,404 filed Jan. 29, 2002, U.S. Provisional Application No. 60/333,620 filed Nov. 26, 2001, and U.S. Provisional Application No. 60/295,961 filed Jun. 4, 2001, which are hereby incorporated in their entirety by reference.

### BACKGROUND OF THE INVENTION

1. Field of the Invention

The present invention relates generally to gene expression and protein production and, more specifically, to compositions and methods for the overexpression of recombinant proteins. Such compositions and methods are useful in the high-level, large-scale production of recombinant proteins.

2. Description of Related Art

A major goal of the biotechnology industry is the development of stable cell-line based systems for the large-scale expression of recombinant proteins such as, e.g., recombinant antibodies. Standard methodologies require time consuming and labor intensive development of suitable recombinant host cell-lines. Conventionally, cells, such as, e.g., CHO-K1 or CHO DUX, are grown in the presence of fetal bovine serum and transfected by the expression vector of interest. The entire population of cells subsequently undergoes a process of selection to remove cells that failed to take up the expression vector. The vector containing pool is then, typically, subcloned and screened for high-level expression. Each of the resulting high-level expressing clones is then expanded and slowly adapted to serum-free, suspension culture which adaptation often results in the loss of expression of the recombinant protein and/or polypeptide.

In addition to these general limitations in recombinant protein expression, efficient functional expression of multi-subunit proteins, such as, e.g., antibodies, requires appropriately balanced expression of both subunit chains. For example, traditional methodologies for the expression of antibody heavy and light chains rely on the co-transfection of plasmids independently carrying a heavy and light chain coding region makes the maintenance of an equal copy number difficult and provides the potential for transcriptional interference between the genes if the vectors integrate close to one another in the genome.

Thus, in spite of considerable research, there remains a need in the art for improved compositions and methods for high-level, large-scale expression of recombinant proteins and/or polypeptides including antibody heavy and light chains. The present invention fulfills these needs and further provides other related advantages by utilizing host cell-lines that are pre-adapted for serum-free, suspension culture in combination with suitable expression vectors for recombinant protein expression. Also provided herein are bi-directional UCOE vectors that permit the simultaneous, high-level expression of two or more recombinant proteins and/or polypeptides from a single UCOE based plasmid vector.

### SUMMARY OF THE INVENTION

The present invention is directed, generally, to compositions and methods for the rapid and efficient development of recombinant cell-lines that are suitable for high-level, large-scale development and manufacture of recombinant proteins and/or polypeptides.

In one aspect, the present invention provides compositions, comprising: (a) an immortalized host cell-line, capable of continuous growth in culture, which host cell-line is capable of growth in serum-free suspension culture, and (b) a vector for sustained overexpression of a recombinant protein and/or polypeptide, such as a UCOE-based vector described herein.

The present invention, in another aspect, provides methods for the high-level, large-scale production of polypeptides. Particular methods comprise the steps of (a) obtaining an immortalized host cell-line capable of growth in suspension; (b) adapting the host cell-line for growth in serum-free medium; (c) transfecting the resulting immortalized host cell-line capable of growth in suspension and serum-free medium with a vector suitable for overexpression of a recombinant protein and/or polypeptide.

According to the compositions and methods of the present invention, suitable immortalized host cell-lines may possess one or more of the following properties: (a) doubling times of no more than 16 hours, preferably between 12 and 16 hours; (b) transfection efficiency of at least 70%, preferably at least 75%, 80%, 85%, 90% or 95%; (c) susceptible to standard selection agents such as, for example, hygromycin, G418, and puromycin; (d) absence of gal-gal glycosylation of recombinant protein and/or polypeptide.

Exemplary immortalized host cell-lines that may be adapted for use in the presently claimed invention include, but are not limited to, the following commercially available host cell-lines: (a) CHO-S (a Chinese hamster ovary host cell-line); (b) 293-F (a human host cell-line); (c) 293-H (a human host cell-line); (d) COS-7L (a monkey host cell-line); (e) D.Mel-2 (an insect host cell-line); (f) Sf21 (an insect host cell-line); and (g) Sf9 (an insect host cell-line). Alternatively, suitable host cell-lines may be obtained through routine experimentation following the methodologies disclosed herein.

Vectors for overexpression of recombinant proteins and/or polypeptides suitable for use in the compositions and methods of the present invention may possess one or more of the following properties: (a) contains one or more elements that facilitate high-level, large-scale expression in the immortalized host cell-line and (b) are resistant to repression of the recombinant protein and/or polypeptide.

Within certain embodiments, vectors of the present invention may further comprise one or more universal chromatin opening elements (UCOEs) as defined herein below. Additionally or alternatively, vectors as disclosed herein may comprise one or more transcriptional promoters such as, for example, the CMV promoter.

Preferred compositions and methods of the present invention are capable of achieving expression levels of at least 50 mg recombinant protein and/or polypeptide per liter of culture, more preferably at least 100 mg recombinant protein and/or polypeptide per liter, and still more preferably at least 200 mg recombinant protein and/or polypeptide per liter.

The present invention further provides compositions and methods that are capable of scale-up to at least 100 liter scale with yields (per 100 liter culture) of at least 1 gram of protein and/or polypeptide, more preferably at least 5 grams of protein and/or polypeptide, still more preferably at least 10 grams of protein and/or polypeptide, and most preferably at least 20 grams of protein and/or polypeptide.

The present invention still further provides compositions and methods employing bi-directional vector systems for the high-level expression of two or more recombinant proteins on a single UCOE-based plasmid vector. Exemplary bi-directional vector systems may comprise one or more transcriptional promoter selected from the group consisting of the murine CMV promoter, the human CMV promoter, and the human beta-actin promoter.

The present invention also provides compositions and methods for improved expression of one or more recombinant protein comprising an RNP UCOE-based plasmid vector, such as, e.g., CET720GFP, optionally comprising one or more deletions within the 8 kb RNP UCOE portion. Illustrative UCOE deletion constructs will preferably retain significant UCOE activity, e.g., at least about 50%, preferably at least about 75%, and more preferably at least 90% or more of UCOE activity relative to the activity of the 8 kb RNP UCOE element described herein. Exemplary deletions may, optionally, comprise deletions within regions of the RNP UCOE selected from the group consisting of ΔBS, ΔEcoNI, ΔEM, ΔMluI, and ΔRV, as depicted in Table 4 and FIG. 14. Deletions within the scope of the present invention are preferably at least 100 bp, more preferably at least 250 bp, still more preferably at least 1000 bp, still more preferably at least 2500 bp and still more preferably at least 4000 bp. Particularly illustrative UCOE vectors of the present invention will thus minimally comprise at least one or more UCOE portions, wherein the UCOE portions retain a desired level of UCOE activity. In one illustrative embodiment, at least about a 4.1 kb UCOE portion corresponding to nucleotide residues 5152-9254 of CET720GFP (SEQ ID NO: 9) is employed. This UCOE portion, for example, has been demonstrated herein to retain a level of UCOE activity comparable to that observed the full 8 kb UCOE element corresponding to nucleotide residues 2225-10525 of CET720GFP (SEQ ID NO: 9). These and other UCOE portions can be readily identified, and their activities evaluated, via routine and art-recognized techniques in view of the disclosure provided herein.

These and other aspects of the present invention will become apparent upon reference to the following detailed description and attached drawings. All references disclosed herein are hereby incorporated by reference in their entirety as if each was incorporated individually.

### BRIEF DESCRIPTION OF THE DRAWINGS AND SEQUENCE IDENTIFIERS

FIG. 1 is a diagrammatic representation of UCOE-based antibody expression cassettes.

FIGS. 2A and 2B are plasmid maps of vectors that may be used for expression of recombinant human antibodies. FIG. 2A shows a plasmid for expression of recombinant human Ig heavy chain. FIG. 2B shows a plasmid for expression of recombinant human Ig kappa light chain.

FIG. 3 is a graph depicting antibody expression levels in CHO cells transfected with and without UCOEs.

FIG. 4 shows the results of scale-up of a CHO-S cell line transfected with vectors expressing the Heavy and Light chains of antibody Ab1 in shake-flask culture and in a 2 liter bioreactor. The left-hand panel shows antibody titer determined by ELISA. The right-hand panel shows cell growth.

FIG. 5 is a graph depicting the levels of Gal-Gal residues on the surface of murine hybridoma, CHO-K1, and CHO-S cells.

FIG. 6 is a diagrammatic representation of the bi-directional UCOE plasmid vector pBDUneo100.

FIG. 7 is a diagrammatic representation of the bi-directional UCOE plasmid vector pBDUneo200.

FIG. 8 is a diagrammatic representation of the bi-directional UCOE plasmid vector pBDUpuro300.

FIG. 9 is a diagrammatic representation of the bi-directional UCOE plasmid vector pBDUpuro400.

FIG. 10 is a diagrammatic representation of the bi-directional UCOE plasmid vector pBDUneo500.

FIG. 11 is a diagrammatic representation of the bi-directional UCOE plasmid vector pBDUneo600.

FIG. 12 is a diagrammatic representation of the bi-directional UCOE plasmid vector pBDUpuro700.

FIG. 13 is a diagrammatic representation of the bi-directional UCOE plasmid vector pBDUpuro800.

FIG. 14 is a diagrammatic representation of deletions within the 8 kb RNP UCOE of CET720GFP.

FIG. 15 is a diagrammatic representation of the bi-directional UCOE plasmid vector pBDUpuro350.

FIG. 16 is a diagrammatic representation of the bi-directional UCOE plasmid vector pBDUpuro450.

FIG. 17 is a diagrammatic representation of the bi-directional UCOE plasmid vector pBDUneo1200.

FIG. 18 is a diagrammatic representation of the bi-directional UCOE plasmid vector pBDUpuro1450.

FIG. 19 is a diagrammatic representation of the bi-directional UCOE plasmid vector pBDUneo1600.

FIG. 20 is a diagrammatic representation of the bi-directional UCOE plasmid vector pBDUpuro1800.

FIG. 21 is a graph depicting the antibody production rates for illustrative cell lines containing bi-directional UCOE plasmid vectors.

### BRIEF DESCRIPTION OF THE SEQUENCE IDENTIFIERS

SEQ ID NO:1 is the polynucleotide sequence of pBDUneo100.

SEQ ID NO:2 is the polynucleotide sequence of pBDUneo200.

SEQ ID NO:3 is the polynucleotide sequence of pBDUpuro300.

SEQ ID NO:4 is the polynucleotide sequence of pBDUpuro400.

SEQ ID NO: 5 is the polynucleotide sequence of pBDUneo500.

SEQ ID NO: 6 is the polynucleotide sequence of pBDUneo600

SEQ ID NO: 7 is the polynucleotide sequence of pBDUpuro700.

SEQ ID NO: 8 is the polynucleotide sequence of pBDUpuro800.

SEQ ID NO: 9 is the polynucleotide sequence of vector CET720GFP.

SEQ ID NOs: 10-26 represent illustrative primer sequences employed in Example 4 for the production of improved UCOE vectors according to the invention.

SEQ ID NO: 27 is the polynucleotide sequence of pBDUpuro350.

SEQ ID NO: 28 is the polynucleotide sequence of pBDUpuro450.

SEQ ID NO: 29 is the polynucleotide sequence of pBDUneo1200.

SEQ ID NO: 30 is the polynucleotide sequence of pBDUpuro1450.

SEQ ID NO: 31 is the polynucleotide sequence of pBDUneo1600.

SEQ ID NO: 32 is the polynucleotide sequence of pBDUpuro1800.

### DETAILED DESCRIPTION OF THE INVENTION

The present invention is directed generally to compositions and methods for use in high-level, large-scale production of recombinant proteins and/or polypeptides. As described further below, illustrative compositions of the present invention include, but are not restricted to, immortalized, serum-free, suspension host cell-lines in combination with one or more expression vectors suitable for the high-level, large-scale expression of recombinant proteins and or polypeptides.

The practice of the present invention will employ, unless indicated specifically to the contrary, conventional methods of virology, immunology, microbiology, molecular biology and recombinant DNA techniques within the skill of the art, many of which are described below for the purpose of illustration. Such techniques are explained fully in the literature. See, e.g., Sambrook, et al. Molecular Cloning: A Laboratory Manual (2nd Edition, 1989); Maniatis et al. Molecular Cloning: A Laboratory Manual (1982); DNA Cloning: A Practical Approach, vol. I & II (D. Glover, ed.); Oligonucleotide Synthesis (N. Gait, ed., 1984); Nucleic Acid Hybridization (B. Hames & S. Higgins, eds., 1985); Transcription and Translation (B. Hames & S. Higgins, eds., 1984); Animal Cell Culture (R. Freshney, ed., 1986); Perbal, A Practical Guide to Molecular Cloning (1984).

All publications, patents and patent applications cited herein, whether supra or infra, are hereby incorporated by reference in their entirety.

As used in this specification and the appended claims, the singular forms “a,” “an” and “the” include plural references unless the content clearly dictates otherwise.

Preparation and Selection of Serum-free, Suspension Host Cell-lines

Host cell-lines ideally suitable for use in the compositions and methods of the present invention may have one or more of the following attributes: (a) capable of immortal, continuous growth in culture; (b) adapted for growth in suspension; (c) rapid growth, preferably 12-16 hour doubling time; (d) high transfection efficiency, preferably at least 70%; (e) susceptibility to selection by standard selection agents, preferably hygromycin, G418 or puromycin; (f) protein glycosylation patterns consistent with use as a human therapeutic, preferably the absence of gal-gal glycosylation pattern; and (g) adapted for growth in serum-free medium, preferably chemically-defined, protein-free growth without indirect animal-derived components.

A host cell-line having one or more of these attributes may be used to develop a system for the rapid development of recombinant host cell-lines that may be transferred into development and manufacturing with reduced effort and time as compared to existing methodologies for the high-level, large-scale production of recombinant proteins and/or polypeptides.

For long-term, high-yield production of recombinant proteins, stable expression is generally preferred. For example, cell-lines that stably express a polynucleotide of interest may be transfected using expression vectors which may contain endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells that successfully express the introduced sequences. Resistant clones of stably transformed cells may be proliferated using tissue culture techniques appropriate to the cell type.

Any number of selection systems may be used to recover transformed cell-lines. These include, but are not limited to, the herpes simplex virus thymidine kinase (Wigler, M. et al. (1977) Cell 11:223-32) and adenine phosphoribosyltransferase (Lowy, I. et al. (1990) Cell 22:817-23) genes which can be employed in tk.sup.- or aprt.sup.-cells, respectively. Also, antimetabolite, antibiotic or herbicide resistance can be used as the basis for selection; for example, dhfr which confers resistance to methotrexate (Wigler, M. et al. (1980) Proc. Natl. Acad. Sci. 77:3567-70); glutamine synthetase (GS) which confers glutainine—independent growth and resistance to methionine sulphoximine (Bebbington et al. (1992) Biotechnology 10(2):169-75; and Cockett et al. (1991) Nucleic Acids Res. 25;19(2):319-25; npt, which confers resistance to the aminoglycosides, neomycin and G-418 (Colbere-Garapin, F. et al (1981) J. Mol. Biol. 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan (1988) Proc. Natl. Acad. Sci. 85:8047-51). The use of visible markers has gained popularity with such markers as anthocyanins, beta-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells that contain and express a desired polynucleotide sequence may be identified by a variety of procedures known to those of skill in the art. These procedures include, but are not limited to, DNA-DNA or DNA-RNA hybridizations and protein bioassay or immunoassay techniques which include, for example, membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton, R. et al. (1990; Serological Methods, a Laboratory Manual, APS Press, St Paul. Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen) between the purification domain and the encoded polypeptide may be used to facilitate purification. One such expression vector provides for expression of a fusion protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath, J. et al. (1992, Prot. Exp. Purif. 3:263-281) while the enterokinase cleavage site provides a means for purifying the desired polypeptide from the fusion protein. A discussion of vectors which contain fusion proteins is provided in Kroll, D. J. et al. (1993; DNA Cell Biol. 12:441-453).

Serum-free, immortal host cell-lines are readily available from a variety of public and/or commercial sources such as, for example, the American Type Culture Collection (ATCC; Manassas, Va.); Celox (St. Paul, Minn.); Invitrogen (Carlsbad, Calif.); the European and Japanese Cell Banks (ECACC, Salisbury, Wiltshire (UK) and JCRB, Shinjuky, Japan, respectively).

Suitable host cell-lines may be obtained by selecting an existing host cell-line that possesses one or more of the above attributes and adapt and/or select for variants of that host cell-line to obtained the remaining attributes. The use of pre-adapted host cell-lines ensures that the cells are capable of achieving the desired conditions prior to beginning the process of transfection and recombinant protein expression. As noted below, such cell-lines are ideally suited for use in conjunction with UCOE containing expression vectors because these vector systems are characterized by stable, long-term, high-level protein expression.

Exemplary suitable host cell-lines that may be modified and/or adapted for use according to the compositions and methods of the present invention include, but are not limited to, the following: (a) 293-F, a human host cell-line; (b) 293-H, a human host cell-line; (c) COS-7L, a monkey host cell-line; (d) D.MEL-2, an insect host cell-line; (e) SF21, an insect host cell-line; (f) SF9, an insect host cell-line; and (g) CHO-S, a Chinese hamster ovary host cell-line.

For example, a Chinese hamster ovary subcloned (CHO-S; Invitrogen/Gibco) that has been adapted to a commercially available chemically defined, protein free media may be suitably employed in the compositions and methods of the present invention. See, D'Anna et al., Radiation Research 148:260-271 (1997); D'Anna et al., Methods in Cell Science 18:115-125 (19960; Deaven et al., Chromosoma 41:129-144 (1973); Gorfein et al., Animal Cell Technology: Basic & Applied Aspects 9:247-252 (Kluwer Academic Publishers, Netherlands, 1998). The CHO-S host cell-line has a 12 to 16 hour doubling time in shaker flask cultures reaching a peak cell density of 9-11×10⁶ viable cells/ml. They are susceptible to hygromycin at 400 ug/ml and geneticin (G418) at 600 ug/ml. The cells grow as attachment independent single cells even in a stationary culture.

The presence of the Galα1→3Galβ1→4GlcNAc-R (Gal-Gal) carbohydrate residue on recombinant proteins used clinically has been associated with rapid protein clearance from the serum. Rodent cells typically introduce the terminal Gal-Gal disaccharide into the carbohydrate structures of secreted glycoproteins although the Gal-Gal residue is not found in human glycoproteins. As a result, the ability to produce recombinant protein without this particular carbohydrate structure is advantageous.

The CHO-S host cell-line is particularly well suited for use in conjunction with expression vectors comprising one or more UCOE elements, as noted herein below. This host cell-line possesses favorable growth characteristics and generates undetectable levels of the Gal-Gal carbohydrate moiety in its surface glycoproteins. Thus, the CHO-S host cell-line is suitable for expression of recombinant proteins and/or polypeptides produced for clinical use.

Preparation and Selection of Expression Vectors

Suitable vector systems for expression of recombinant proteins and/or polypeptides according to the present invention may include one or more of the following attributes: (a) ease of manipulation; (b) elements that make high-level expression site-of-integration independent; (c) elements that make expression resistant to silencing/repression thereby allowing for sustained, stable expression over long periods of time; and (d) elements that express at high-levels in different cell types and in different species.

In order to express a desired protein and/or polypeptide, the nucleotide sequences encoding the polypeptide, or functional equivalents, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described, for example, in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.

A variety of expression vector/host systems may be utilized to contain and express polynucleotide sequences. These include, but are not limited to plasmid or cosmid DNA expression vectors; insect cell systems infected with virus expression vectors (e.g., baculovirus); plant cell systems transformed with virus expression vectors (e.g., cauliflower mosaic virus, CaMV; tobacco mosaic virus, TMV); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. In mammalian cell systems, promoters from mammalian genes or from mammalian viruses are generally preferred. If it is necessary to generate a cell-line that contains multiple copies of the sequence encoding a polypeptide, vectors containing GS or DHFR selectable markers or vectors based on SV40 or EBV may be advantageously used with an appropriate selectable marker.

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard, E. K. et al. (1994) Proc. Natl. Acad. Sci. 91 :3224-3227).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

Exemplary preferred elements suitable for making high-level expression site-of-integration independent include, for example, universal chromatin opening elements (UCOEs). UCOEs are polynucleotide sequences that maintain chromatin in an “open” configuration. See, e.g., Crombie et al., PCT Patent Application No. WO0005393 (2000). Inclusion of a UCOE in an expression vector upsteam of the promoter provides high-levels of expression that are independent of integration site and are resistant to silencing. Efficient expression can be derived from a single copy of an integrated gene site resulting in a higher percentage of cells expressing the marker gene in the selected pool in comparison to standard non-UCOE containing vectors. This, in combination with the utilization of a serum free, suspension adapted parent cell-line allows for rapid production of large quantities of protein in a short period of time. The increased efficiency obtained with the UCOE vector significantly reduces the number of transfectants which need to be screened in order to obtain a high productivity subclone.

Utilization of vectors containing one or more UCOEs in a suspension-adapted host cell-line allows for rapid development and scale-up for production protein and/or polypeptide such as, for example, antibody or fragment thereof. UCOEs allow for screening of a small number of subclones to obtain a clone capable of producing at least 50 mg/L of protein and/or polypeptide, more preferably at least 100 mg/L of protein and/or polypeptide, and still more preferably at least 200 mg/L of protein and/or polypeptide in a 5 week period in serum free conditions.

Preferably, expression vector systems suitable for use in the compositions and methods of the present invention are capable of yielding expression levels in excess of 1 g protein and/or polypeptide per liter of suspension culture. More preferably, expression vectors are capable of use in stable host cell-lines wherein least 20 pg protein and/or polypeptide per cell are achieved per day.

As discussed in detail herein below, within certain embodiments of the present invention, the protein and/or polypeptide may comprise one or more subunits such as, for example, antibody heavy and light chains or fragments thereof. As is well understood in the art, efficient functional antibody production requires appropriately balanced expression of the heavy and light chains. Transfection of the two chains on separate plasmids makes maintenance of an equal copy number difficult and provides the potential for transcriptional interference between the genes if the vectors integrate close to one another in the genome. Consequently, bi-directional vectors for the co-expression of two genes on the same vector may be employed. As disclosed in further detail in the Examples herein below, exemplary bi-directional UCOE-based vector systems, within the scope of the present invention, may, optionally, be constructed based on the “hybrid” RNP/beta-actin UCOE (Cobra Therapeutics). Vectors may comprise one or more antibiotic resistance markers such as, e.g., the neomycin or puromycin resistance markers, and/or may comprise one or more mammalian promoter such as, e.g., the murine CMV promoter (mCMV), the human CMV promoter (hCMV), or the human actin promoters to drive light or heavy chain expression.

Transfection of Host Cell-lines with Expression Vectors of the Present Invention

Transfection of a standard host cell-line, preadapted to grow in a large scale setting, allows for more rapid cell-line development thereby increasing the transition rate from research into development and manufacturing. In contrast, the traditional approach of using a parent cell-line which requires serum free and suspension adaptation after transfection further increases the need for screening a large number of subclones, because many of the subclones will not be able to grow under conditions that allow large scale protein production. Use of a preadapted cell-line can reduce the time required to develop a cell-line from months to weeks. The cell-line is preadapted to a chemically defined, protein free media and grows rapidly to high cell densities in a shaker flask or bioreactor.

Suitable transfection protocols are readily known and/or available to those of skill in the art. Exemplary transfection protocols that are suitable for achieving high-level, large-scale transfection are those recommended by Invitrogen/Gibco for transfection of the CHO-S host cell-line. Generally, positive selection of transfected cells may be achieved using agents such as, for example, hygromycin, G418, and puromycin. Transfection efficiencies are typically at least 70%, more preferably at least 75%, 80%, 85%, 90% or 95%. Following transfection and selection, the pool of resulting clones may, optionally, be further subcloned to identify individual clones with the highest levels of protein expression.

Selection of Cell Culture Conditions

Selection and testing of serum-free media suitable for culture of the immortalized suspension cells according to the present invention may be achieved by the skilled artisan by routine experimentation. For CHO-S cells, described herein above, the CD-CHO media is suitable. (e.g, available from Invitrogen or Gibco).

Exemplary Proteins and/or Polypeptides Suitable for High-level, Large-scale Expression

As used herein, the terms “protein” and “polypeptide” are used in their conventional meaning, i.e., as a sequence of amino acids. The polypeptides are not limited to a specific length of the product; thus, peptides, oligopeptides, and proteins are included within the definition of polypeptide, and such terms may be used interchangeably herein unless specifically indicated otherwise. This term also does not refer to or exclude post-expression modifications of the polypeptide, for example, glycosylations, acetylations, phosphorylations and the like, as well as other modifications known in the art, both naturally occurring and non-naturally occurring. As noted above, however, preferred proteins and/or polypeptides according to the present invention lack Gal-Gal glycosylation. A polypeptide may be an entire protein, or a subsequence thereof. Particular polypeptides of interest in the context of this invention are amino acid subsequences comprising epitopes, i.e., antigenic determinants substantially responsible for the immunogenic properties of a polypeptide and being capable of evoking an immune response.

In certain preferred embodiments, the polypeptides produced and/or employed according to the present invention are immunogenic, i.e., they react detectably within an immunoassay (such as an ELISA or T-cell stimulation assay) with antisera and/or T-cells from a patient with a cancer. Screening for immunogenic activity can be performed using techniques well known to the skilled artisan. For example, such screens can be performed using methods such as those described in Harlow and Lane, Antibodies: A Laboratory Manual, Cold Spring Harbor Laboratory, 1988. In one illustrative example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

As would be recognized by the skilled artisan, immunogenic portions of the polypeptides produced according to the disclosure provided herein are also encompassed by the present invention. An “immunogenic portion,” as used herein, is a fragment of an immunogenic polypeptide of the invention that itself is immunologically reactive (i.e., specifically binds) with the B-cells and/or T-cell surface antigen receptors that recognize the polypeptide. Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell-lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well-known techniques.

In one preferred embodiment, an immunogenic portion of a polypeptide of the present invention is a portion that reacts with antisera and/or T-cells at a level that is not substantially less than the reactivity of the full-length polypeptide (e.g., in an ELISA and/or T-cell reactivity assay). Preferably, the level of immunogenic activity of the immunogenic portion is at least about 50%, preferably at least about 70% and most preferably greater than about 90% of the immunogenicity for the full-length polypeptide. In some instances, preferred immunogenic portions will be identified that have a level of immunogenic activity greater than that of the corresponding full-length polypeptide, e.g., having greater than about 100% or 150% or more immunogenic activity.

In certain other embodiments, illustrative immunogenic portions may include peptides in which an N-terminal leader sequence and/or transmembrane domain have been deleted. Other illustrative immunogenic portions will contain a small N- and/or C-terminal deletion (e.g., 1-30 amino acids, preferably 5-15 amino acids), relative to the mature protein.

In another embodiment, a protein and/or polypeptide made and/or used according to the present invention may also comprise one or more polypeptides that are immunologically reactive with T cells and/or antibodies generated against a polypeptide of the invention, particularly a polypeptide having an amino acid sequence disclosed herein, or to an immunogenic fragment or variant thereof.

A polypeptide “variant,” as the term is used herein, is a polypeptide that typically differs from a polypeptide specifically disclosed herein in one or more substitutions, deletions, additions and/or insertions. Such variants may be naturally occurring or may be synthetically generated, for example, by modifying one or more of the above polypeptide sequences of the invention and evaluating their activity as described herein and/or using any of a number of techniques well known in the art. Illustrative variant sequences according to the present invention are those sequences related by homology to the 8 kb RNP UCOE sequence provided herein, or a subsequence thereof, which retain a desired degree of UCOE activity.

In one embodiment, for example, particularly illustrative variant sequences of the invention comprise polynucleotide sequences having at least 70%, 75%, 80%, 85%, 90%, 95% or 99% or more identity with a UCOE polynucleotide specifically disclosed herein. Preferably such variants exhibit at least 70%, 75%, 80%, 85%, 90%, 95% or 100% or more UCOE activity when compared with the UCOE activity exhibited by the 8 kb RNP UCOE element disclosed herein.

In many instances, a variant will contain conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. As described above, modifications may be made in the structure of the polynucleotides and polypeptides of the present invention and still obtain a functional molecule that encodes a variant or derivative polypeptide with desirable characteristics, e.g., with immunogenic characteristics. When it is desired to alter the amino acid sequence of a polypeptide to create an equivalent, or even an improved, variant or portion of a polypeptide of the invention, one skilled in the art will typically change one or more of the codons of the encoding DNA sequence according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated that various changes may be made in the peptide sequences of the disclosed compositions, or corresponding DNA sequences which encode said peptides without appreciable loss of their biological utility or activity.

TABLE 1

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

In addition, any polynucleotide may be further modified to increase stability in vivo. Possible modifications include, but are not limited to, the addition of flanking sequences at the 5′ and/or 3′ ends; the use of phosphorothioate or 2′ O-methyl rather than phosphodiesterase linkages in the backbone; and/or the inclusion of nontraditional bases such as inosine, queosine and wybutosine, as well as acetyl-methyl-, thio- and other modified forms of adenine, cytidine, guanine, thymine and uridine.

Amino acid substitutions may further be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

When comparing polypeptide sequences, two sequences are said to be “identical” if the sequence of amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Saitou, N. Nei, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al. (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment.

In one preferred approach, the “percentage of sequence identity” is determined by comparing two optimally aligned sequences over a window of comparison of at least 20 positions, wherein the portion of the polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical amino acid residue occurs in both sequences to yield the number of matched positions, dividing the number of matched positions by the total number of positions in the reference sequence (i.e., the window size) and multiplying the results by 100 to yield the percentage of sequence identity.

Within other illustrative embodiments, a polypeptide produced and/or employed according to the present invention may be a xenogeneic polypeptide that comprises a polypeptide having substantial sequence identity, as described above, to the human polypeptide (also termed autologous antigen) which served as a reference polypeptide, but which xenogeneic polypeptide is derived from a different, non-human species. One skilled in the art will recognize that “self” antigens are often poor stimulators of CD8+ and CD4+ T-lymphocyte responses, and therefore efficient immunotherapeutic strategies directed against tumor polypeptides require the development of methods to overcome immune tolerance to particular self tumor polypeptides. For example, humans immunized with prostase protein from a xenogeneic (non human) origin are capable of mounting an immune response against the counterpart human protein, e.g. the human prostase tumor protein present on human tumor cells. Therefore, one aspect of the present invention provides xenogeneic variants of the protein and/or polypeptides described herein.

More particularly, the invention is directed to mouse, rat, monkey, porcine and other non-human polypeptides which can be used as xenogeneic forms of human polypeptides set forth herein.

Within other illustrative embodiments, the present invention may employ and/or produce a fusion polypeptide that comprises multiple polypeptides and/or polypeptide subunits, as described herein, or that comprises at least one polypeptide as described herein and an unrelated sequence. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the polypeptide or to enable the polypeptide to be targeted to desired intracellular compartments. Still further fusion partners include affinity tags, which facilitate purification of the polypeptide.

Fusion polypeptides may generally be prepared using standard techniques, including chemical conjugation. Preferably, a fusion polypeptide is expressed as a recombinant polypeptide employing compositions and methods of the present invention, and allowing the production of increased levels in an expression system. Briefly, for example, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion polypeptide that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion polypeptide using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. No. 4,935,233 and U.S. Pat. No. 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

The fusion polypeptide can comprise a polypeptide made and/or described herein together with an unrelated protein, such as an immunogenic protein capable of eliciting a recall response. Examples of such proteins include tetanus, tuberculosis and hepatitis proteins (see, for example, Stoute et al. New Engl. J. Med., 336:86-91, 1997).

In one preferred embodiment, the immunological fusion partner is derived from a Mycobacterium sp., such as a Mycobacterium tuberculosis-derived Ra12 fragment. Ra12 compositions and methods for their use in enhancing the expression and/or immunogenicity of heterologous polynucleotide/polypeptide sequences is described in U.S. patent application Ser. No. 60/158,585, the disclosure of which is incorporated herein by reference in its entirety. Briefly, Ra12 refers to a polynucleotide region that is a subsequence of a Mycobacterium tuberculosis MTB32A nucleic acid. MTB32A is a serine protease of 32 KD molecular weight encoded by a gene in virulent and avirulent strains of M. tuberculosis. The nucleotide sequence and amino acid sequence of MTB32A have been described (for example, U.S. patent application Ser. No. 60/158,585; see also, Skeiky et al., Infection and Immun. (1999) 67:3998-4007, incorporated herein by reference). C-terminal fragments of the MTB32A coding sequence express at high levels and remain as a soluble polypeptides throughout the purification process. Moreover, Ra12 may enhance the immunogenicity of heterologous immunogenic polypeptides with which it is fused. One preferred Ra12 fusion polypeptide comprises a 14 KD C-terminal fragment corresponding to amino acid residues 192 to 323 of MTB32A. Other preferred Ra12 polynucleotides generally comprise at least about 15 consecutive nucleotides, at least about 30 nucleotides, at least about 60 nucleotides, at least about 100 nucleotides, at least about 200 nucleotides, or at least about 300 nucleotides that encode a portion of a Ra12 polypeptide. Ra12 polynucleotides may comprise a native sequence (i.e., an endogenous sequence that encodes a Ra12 polypeptide or a portion thereof) or may comprise a variant of such a sequence. Ra12 polynucleotide variants may contain one or more substitutions, additions, deletions and/or insertions such that the biological activity of the encoded fusion polypeptide is not substantially diminished, relative to a fusion polypeptide comprising a native Ra12 polypeptide. Variants preferably exhibit at least about 70% identity, more preferably at least about 80% identity and most preferably at least about 90% identity to a polynucleotide sequence that encodes a native Ra12 polypeptide or a portion thereof.

Within other preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion polypeptide. A repeat portion is found in the C-terminal region starting at residue 178. A particularly preferred repeat portion incorporates residues 188-305.

Yet another illustrative embodiment involves fusion polypeptides, and the polynucleotides encoding them, wherein the fusion partner comprises a targeting signal capable of directing a polypeptide to the endosomal/lysosomal compartment, as described in U.S. Pat. No. 5,633,234. An immunogenic polypeptide of the invention, when fused with this targeting signal, will associate more efficiently with MHC class II molecules and thereby provide enhanced in vivo stimulation of CD4⁺ T-cells specific for the polypeptide.

In general, protein and/or polypeptides (including fusion polypeptides) of the invention are isolated. An “isolated” polypeptide is one that is removed from its original environment. For example, a naturally-occurring protein or polypeptide is isolated if it is separated from some or all of the coexisting materials in the natural system. Preferably, such polypeptides are also purified, e.g., are at least about 90% pure, more preferably at least about 95% pure and most preferably at least about 99% pure.

Particularly preferred polypeptides produced by the methods of the present invention include binding agents, such as antibodies and antigen-binding fragments thereof, that exhibit immunological binding to a target polypeptide of interest, such as a polypeptide associated with a particular disease state, or to a portion, variant or derivative thereof. An antibody, or antigen-binding fragment thereof, is said to “specifically bind,” “immunogically bind,” and/or is “immunologically reactive” to a polypeptide of the invention if it reacts at a detectable level (within, for example, an ELISA assay) with the polypeptide, and does not react detectably with unrelated polypeptides under similar conditions.

Immunological binding, as used in this context, generally refers to the non-covalent interactions of the type which occur between an immunoglobulin molecule and an antigen for which the immunoglobulin is specific. The strength, or affinity of immunological binding interactions can be expressed in terms of the dissociation constant (K_{d}) of the interaction, wherein a smaller K_{d} represents a greater affinity. Immunological binding properties of selected polypeptides can be quantified using methods well known in the art. One such method entails measuring the rates of antigen-binding site/antigen complex formation and dissociation, wherein those rates depend on the concentrations of the complex partners, the affinity of the interaction, and on geometric parameters that equally influence the rate in both directions. Thus, both the “on rate constant” (Kₒₙ) and the “off rate constant” (K_{off}) can be determined by calculation of the concentrations and the actual rates of association and dissociation. The ratio of K_{off}/Kₒₙ enables cancellation of all parameters not related to affinity, and is thus equal to the dissociation constant K_{d}. See, generally, Davies et al. (1990) Annual Rev. Biochem. 59:439-473.

An “antigen-binding site,” or “binding portion” of an antibody refers to the part of the immunoglobulin molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains are referred to as “hypervariable regions” which are interposed between more conserved flanking stretches known as “framework regions,” or “FRs”. Thus the term “FR” refers to amino acid sequences which are naturally found between and adjacent to hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.”

Certain binding agents, such as those specific for a tumor-associated protein, will be further capable of differentiating between patients with and without a cancer using the representative assays provided herein and known in the art. For example, antibodies or other binding agents that bind to a tumor protein will preferably generate a signal indicating the presence of a cancer in at least about 20% of patients with the disease, more preferably at least about 30% of patients. Alternatively, or in addition, the antibody will generate a negative signal indicating the absence of the disease in at least about 90% of individuals without the cancer. To determine whether a binding agent satisfies this requirement, biological samples (e.g., blood, sera, sputum, urine and/or tumor biopsies) from patients with and without a cancer (as determined using standard clinical tests) may be assayed as described herein for the presence of polypeptides that bind to the binding agent. Preferably, a statistically significant number of samples with and without the disease will be assayed. Each binding agent should satisfy the above criteria; however, those of ordinary skill in the art will recognize that binding agents may be used in combination to improve sensitivity. Other binding agents produced according to the present invention will also have therapeutic value based on their specificity for tumor-associated polypeptide sequences.

Any agent that satisfies the above requirements may be a binding agent. For example, a binding agent may be a ribosome, with or without a peptide component, an RNA molecule or a polypeptide. In a preferred embodiment, a binding agent is an antibody or an antigen-binding fragment thereof. Antibodies may be prepared by any of a variety of techniques known to those of ordinary skill in the art. See, e.g., Harlow and Lane, Antibodies: A Laboratory Manual, Cold Spring Harbor Laboratory, 1988. In addition to the methods exemplified herein according to the present invention, numerous antibody production techniques are available to the skilled artisan. For example, antibodies can also be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J. Immunol. 6:511-519, 1976, and improvements thereto. Briefly, these methods involve the preparation of immortal cell-lines capable of producing antibodies having the desired specificity (i.e., reactivity with the polypeptide of interest). Such cell-lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell-line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of therapeutically useful molecules are known in the art which comprise antigen-binding sites that are capable of exhibiting immunological binding properties of an antibody molecule. The proteolytic enzyme papain preferentially cleaves IgG molecules to yield several fragments, two of which (the “F(ab)” fragments) each comprise a covalent heterodimer that includes an intact antigen-binding site. The enzyme pepsin is able to cleave IgG molecules to provide several fragments, including the “F(ab′)₂” fragment which comprises both antigen-binding sites. An “Fv” fragment can be produced by preferential proteolytic cleavage of an IgM, and on rare occasions IgG or IgA immunoglobulin molecule. Fv fragments are, however, more commonly derived using recombinant techniques known in the art. The Fv fragment includes a non-covalent V_{H}::V_{L} heterodimer including an antigen-binding site which retains much of the antigen recognition and binding capabilities of the native antibody molecule. Inbar et al. (1972) Proc. Nat. Acad. Sci. USA 69:2659-2662; Hochman et al. (1976) Biochem 15:2706-2710; and Ehrlich et al. (1980) Biochem 19:4091-4096.

A single chain Fv (“sFv”) polypeptide is a covalently linked V_{H}::V_{L} heterodimer which is expressed from a gene fusion including V_{H}- and V_{L}-encoding genes linked by a peptide-encoding linker. Huston et al. (1988) Proc. Nat. Acad. Sci. USA 85(16):5879-5883. A number of methods have been described to discern chemical structures for converting the naturally aggregated—but chemically separated—light and heavy polypeptide chains from an antibody V region into an sFv molecule which will fold into a three dimensional structure substantially similar to the structure of an antigen-binding site. See, e.g., U.S. Pat. Nos. 5,091,513 and 5,132,405, to Huston et al.; and U.S. Pat. No. 4,946,778, to Ladner et al.

Each of the above-described molecules includes a heavy chain and a light chain CDR set, respectively interposed between a heavy chain and a light chain FR set which provide support to the CDRS and define the spatial relationship of the CDRs relative to each other. As used herein, the term “CDR set” refers to the three hypervariable regions of a heavy or light chain V region. Proceeding from the N-terminus of a heavy or light chain, these regions are denoted as “CDR1,” “CDR2,” and “CDR3” respectively. An antigen-binding site, therefore, includes six CDRs, comprising the CDR set from each of a heavy and a light chain V region. A polypeptide comprising a single CDR, (e.g., a CDR1, CDR2 or CDR3) is referred to herein as a “molecular recognition unit.” Crystallographic analysis of a number of antigen-antibody complexes has demonstrated that the amino acid residues of CDRs form extensive contact with bound antigen, wherein the most extensive antigen contact is with the heavy chain CDR3. Thus, the molecular recognition units are primarily responsible for the specificity of an antigen-binding site.

As used herein, the term “FR set” refers to the four flanking amino acid sequences which frame the CDRs of a CDR set of a heavy or light chain V region. Some FR residues may contact bound antigen; however, FRs are primarily responsible for folding the V region into the antigen-binding site, particularly the FR residues directly adjacent to the CDRS. Within FRs, certain amino residues and certain structural features are very highly conserved. In this regard, all V region sequences contain an internal disulfide loop of around 90 amino acid residues. When the V regions fold into a binding-site, the CDRs are displayed as projecting loop motifs which form an antigen-binding surface. It is generally recognized that there are conserved structural regions of FRs which influence the folded shape of the CDR loops into certain “canonical” structures—regardless of the precise CDR amino acid sequence. Further, certain FR residues are known to participate in non-covalent interdomain contacts which stabilize the interaction of the antibody heavy and light chains.

A number of “humanized” antibody molecules comprising an antigen-binding site derived from a non-human immunoglobulin have been described, including chimeric antibodies having rodent V regions and their associated CDRs fused to human constant domains (Winter et al. (1991) Nature 349:293-299; Lobuglio et al. (1989) Proc. Nat. Acad. Sci. USA 86:4220-4224; Shaw et al. (1987) J Immunol. 138:4534-4538; and Brown et al. (1987) Cancer Res. 47:3577-3583), rodent CDRs grafted into a human supporting FR prior to fusion with an appropriate human antibody constant domain (Riechmann et al. (1988) Nature 332:323-327; Verhoeyen et al. (1988) Science 239:1534-1536; and Jones et al. (1986) Nature 321:522-525), and rodent CDRs supported by recombinantly veneered rodent FRs (European Patent Publication No. 519,596, published Dec. 23, 1992). These “humanized” molecules are designed to minimize unwanted immunological response toward rodent antihuman antibody molecules which limits the duration and effectiveness of therapeutic applications of those moieties in human recipients.

As used herein, the terms “veneered FRs” and “recombinantly veneered FRs” refer to the selective replacement of FR residues from, e.g., a rodent heavy or light chain V region, with human FR residues in order to provide a xenogeneic molecule comprising an antigen-binding site which retains substantially all of the native FR polypeptide folding structure. Veneering techniques are based on the understanding that the ligand binding characteristics of an antigen-binding site are determined primarily by the structure and relative disposition of the heavy and light chain CDR sets within the antigen-binding surface. Davies et al. (1990) Ann. Rev. Biochem. 59:439-473. Thus, antigen binding specificity can be preserved in a humanized antibody only wherein the CDR structures, their interaction with each other, and their interaction with the rest of the V region domains are carefully maintained. By using veneering techniques, exterior (e.g., solvent-accessible) FR residues which are readily encountered by the immune system are selectively replaced with human residues to provide a hybrid molecule that comprises either a weakly immunogenic, or substantially non-immunogenic veneered surface.

The process of veneering makes use of the available sequence data for human antibody variable domains compiled by Kabat et al., in Sequences of Proteins of Immunological Interest, 4th ed., (U.S. Dept. of Health and Human Services, U.S. Government Printing Office, 1987), updates to the Kabat database, and other accessible U.S. and foreign databases (both nucleic acid and protein). Solvent accessibilities of V region amino acids can be deduced from the known three-dimensional structure for human and murine antibody fragments. There are two general steps in veneering a murine antigen-binding site. Initially, the FRs of the variable domains of an antibody molecule of interest are compared with corresponding FR sequences of human variable domains obtained from the above-identified sources. The most homologous human V regions are then compared residue by residue to corresponding murine amino acids. The residues in the murine FR which differ from the human counterpart are replaced by the residues present in the human moiety using recombinant techniques well known in the art. Residue switching is only carried out with moieties which are at least partially exposed (solvent accessible), and care is exercised in the replacement of amino acid residues which may have a significant effect on the tertiary structure of V region domains, such as proline, glycine and charged amino acids.

In this manner, the resultant “veneered” murine antigen-binding sites are thus designed to retain the murine CDR residues, the residues substantially adjacent to the CDRs, the residues identified as buried or mostly buried (solvent inaccessible), the residues believed to participate in non-covalent (e.g., electrostatic and hydrophobic) contacts between heavy and light chain domains, and the residues from conserved structural regions of the FRs which are believed to influence the “canonical” tertiary structures of the CDR loops. These design criteria are then used to prepare recombinant nucleotide sequences which combine the CDRs of both the heavy and light chain of a murine antigen-binding site into human-appearing FRs that can be used to transfect mammalian cells for the expression of recombinant human antibodies which exhibit the antigen specificity of the murine antibody molecule.

In another embodiment of the invention, antibodies produced according to the present invention may be coupled to one or more therapeutic agents. Suitable agents in this regard include radionuclides, differentiation inducers, drugs, toxins, and derivatives thereof. Preferred radionuclides include ⁹⁰Y, ¹²³I, ¹²⁵I, ¹³¹I, ¹⁸⁶Re, ¹⁸⁸Re, ²¹¹At, and ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group that is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

Polynucleotides Suitable for Expressing Proteins and/or Polypeptides

The present invention, in other aspects, provides polynucleotides that encode the recombinant proteins and/or polypeptides disclosed herein above. The terms “DNA” and “polynucleotide” are used essentially interchangeably herein to refer to a DNA molecule that has been isolated free of total genomic DNA of a particular species. “Isolated,” as used herein, means that a polynucleotide is substantially away from other coding sequences, and that the DNA molecule does not contain large portions of unrelated coding DNA, such as large chromosomal fragments or other functional genes or polypeptide coding regions. Of course, this refers to the DNA molecule as originally isolated, and does not exclude genes or coding regions later added to the segment by the hand of man.

Polynucleotides may comprise a native sequence (i.e. an endogenous sequence that encodes a protein and/or polypeptide, for example an antibody, or portion thereof) or may comprise a sequence that encodes a variant or derivative, preferably and immunogenic variant or derivative, of such a sequence. In certain embodiments, the polynucleotide sequences may encode immunogenic polypeptides, as described above.

Typically, polynucleotide variants will contain one or more substitutions, additions, deletions and/or insertions, preferably such that the immunogenicity of the polypeptide encoded by the variant polynucleotide is not substantially diminished relative to a polypeptide encoded by a polynucleotide sequence specifically set forth herein). The term “variants” should also be understood to encompass homologous genes of xenogeneic origin.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

Polynucleotides suitable for high-level, large-scale expression according to the present invention may be identified, prepared and/or manipulated using any of a variety of well established techniques (see generally, Sambrook et al., Molecular Cloning: A Laboratory Manual, Cold Spring Harbor Laboratories, Cold Spring Harbor, N.Y., 1989, and other like references). For example, a polynucleotide may be identified by screening a microarray of cDNAs for tumor-associated expression. Such screens may be performed, for example, using the microarray technology of Affymetrix, Inc. (Santa Clara, Calif.) according to the manufacturer's instructions (and essentially as described by Schena et al., Proc. Natl. Acad. Sci. USA 93:10614-10619, 1996 and Heller et al., Proc. Natl. Acad. Sci. USA 94:2150-2155, 1997). Alternatively, polynucleotides may be amplified from cDNA prepared from cells expressing the proteins described herein, such as tumor cells.

Many template dependent processes are available to amplify a target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

Any of a number of other template dependent processes, many of which are variations of the PCR™ amplification technique, are readily known and available in the art. Illustratively, some such methods include the ligase chain reaction (referred to as LCR), described, for example, in Eur. Pat. Appl. Publ. No. 320,308 and U.S. Pat. No. 4,883,750; Qbeta Replicase, described in PCT Intl. Pat. Appl. Publ. No. PCT/US87/00880; Strand Displacement Amplification (SDA) and Repair Chain Reaction (RCR). Still other amplification methods are described in Great Britain Pat. Appl. No. 2 202 328, and in PCT Intl. Pat. Appl. Publ. No. PCT/US89/01025. Other nucleic acid amplification procedures include transcription-based amplification systems (TAS) (PCT Intl. Pat. Appl. Publ. No. WO 88/10315), including nucleic acid sequence based amplification (NASBA) and 3SR. Eur. Pat. Appl. Publ. No. 329,822 describes a nucleic acid amplification process involving cyclically synthesizing single-stranded RNA (“ssRNA”), ssDNA, and double-stranded DNA (dsDNA). PCT Intl. Pat. Appl. Publ. No. WO 89/06700 describes a nucleic acid sequence amplification scheme based on the hybridization of a promoter/primer sequence to a target single-stranded DNA (“ssDNA”) followed by transcription of many RNA copies of the sequence. Other amplification methods such as “RACE” (Frohman, 1990), and “one-sided PCR” (Ohara, 1989) are also well-known to those of skill in the art.

An amplified portion of a polynucleotide of the present invention may be used to isolate a full length gene from a suitable library (e.g., a tumor cDNA library) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences. Alternatively, or in addition, essentially any amplified polynucleotide may be employed in routine subcloning techniques in order to arrive at a UCOE-based vector according to this invention.

For hybridization techniques, a partial sequence may be labeled (e.g., by nick-translation or end-labeling with ³²P) using well known techniques. A bacterial or bacteriophage library is then generally screened by hybridizing filters containing denatured bacterial colonies (or lawns containing phage plaques) with the labeled probe (see Sambrook et al., Molecular Cloning: A Laboratory Manual, Cold Spring Harbor Laboratories, Cold Spring Harbor, N.Y., 1989). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences can then assembled into a single contiguous sequence. A full length cDNA molecule can be generated by ligating suitable fragments, using well known techniques.

Alternatively, amplification techniques, such as those described above, can be useful for obtaining a full length coding sequence from a partial cDNA sequence. One such amplification technique is inverse PCR (see Triglia et al., Nucl. Acids Res. 16:8186, 1988), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al., PCR Methods Applic. 1:111-19, 1991) and walking PCR (Parker et al., Nucl. Acids. Res. 19:3055-60, 1991). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

In certain preferred embodiments of the invention, polynucleotide sequences or fragments thereof are employed in the construction and/or use of UCOE-based vectors and encode one or more polypeptides of interest, such as antibodies or fusion proteins or functional equivalents thereof. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, and/or expression of the gene product. For example, DNA shuffling by random fragmentation and PCR reassembly of gene fragments and synthetic oligonucleotides may be used to engineer the nucleotide sequences. In addition, site-directed mutagenesis may be used to insert new restriction sites, alter glycosylation patterns, change codon preference, produce splice variants, or introduce mutations, and so forth.

A newly synthesized peptide may be substantially purified, for example, by preparative high performance liquid chromatography (e.g., Creighton, T. (1983) Proteins, Structures and Molecular Principles, WH Freeman and Co., New York, N.Y.) or other comparable techniques available in the art. The composition of the synthetic peptides may be confirmed by amino acid analysis or sequencing (e.g., the Edman degradation procedure). Additionally, the amino acid sequence of a polypeptide, or any part thereof, may be altered during direct synthesis and/or combined using chemical methods with sequences from other proteins, or any part thereof, to produce a variant polypeptide.

The following Examples are offered by way of illustration not limitation.

### EXAMPLES

### Example 1

### Expression of Recombinant Antibody in a UCOE-Based Expression Vector System

This example discloses a comparison between the expression levels of recombinant antibodies using vectors with and without UCOEs.

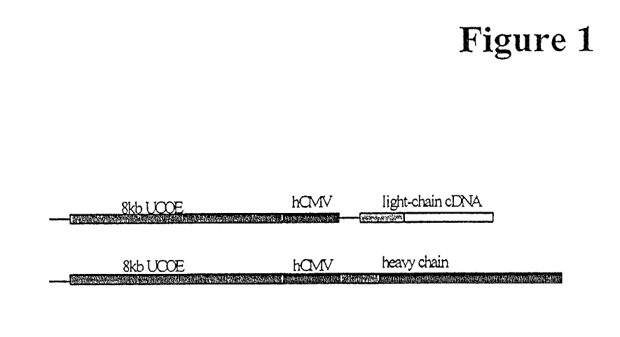

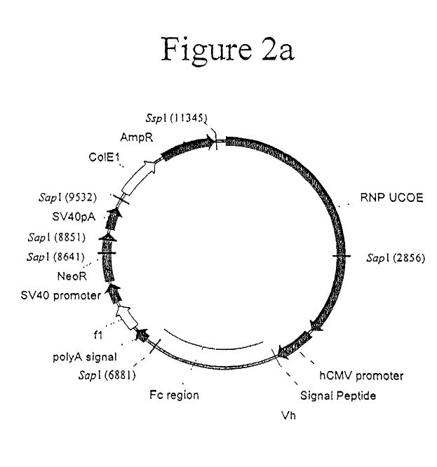

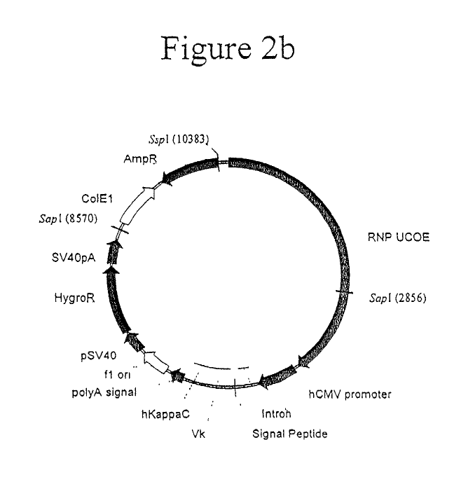

Engineered human antibody Ab3 was expressed from vectors containing a human RNP UCOE as shown in FIG. 1. Identical vectors, but without the UCOE element, were also constructed. The Ig heavy chain coding sequence in this example comprises an engineered human V-region sequence introduced upstream of and in frame with a genomic DNA fragment encoding a human Ig gamma-1 constant region. The Ig light chain coding sequence comprises an engineered human V-region sequence introduced upstream of and in frame with a cDNA fragment encoding a human Ig kappa constant region. The vector for expression of the Ig heavy chain additionally contains a neo selectable marker gene and the vector for expression of the Ig light chain contains a hygromycin selectable marker. See FIG. 2A.

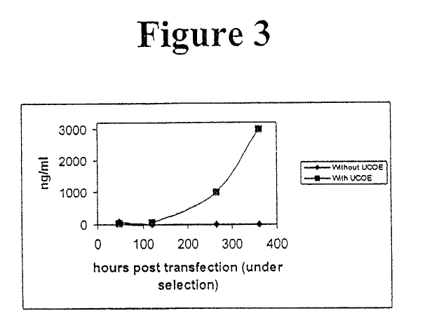

CHO-K1 cells were co-transfected with the light-chain and heavy-chain vectors using lipofectamine (Life Technologies) according to the manufacturers' instructions. Cells were selected using hygromycin and G418. Pools of transfectants were maintained and levels of assembled immunoglobulin secreted into culture medium were determined by ELISA at various times post-transfection. (FIG. 3). In the absence of the RNP UCOE, antibody expression levels were low (approximately 48 ng/ml) 48 hours after transfection and declined thereafter. In contrast, in transfection pools from expression vectors containing the RNP UCOE, antibody levels continued to accumulate as the transfected cultures were expanded, reaching 3 micrograms/ml 15 days post-transfection. Thus, use of UCOEs permited rapid generation of pools of transfected cells that express high levels of recombinant immunoglobulin.

### Example 2

### High-level, Large-scale Expression Achieved in CHO Host Cell-line Transfected with UCOE-Based Expression Vector System

CHO-S cells were co-transfected with vectors containing UCOE antibody expression cassettes (shown in FIG. 1) to produce the engineered human antibody Ab1. The Ig heavy chain coding sequence comprises an engineered human V-region sequence introduced upstream of and in frame with a genomic DNA fragment encoding a human Ig gamma-4 constant region. The Ig light chain coding sequence comprises an engineered human V-region sequence introduced upstream of and in frame with a cDNA fragment encoding a human Ig kappa constant region. The vector for expression of the Ig Heavy chain additionally contains a neo selectable marker gene and the vector for expression of the Ig light chain contains a hygromycin selectable marker. See FIG. 2B.

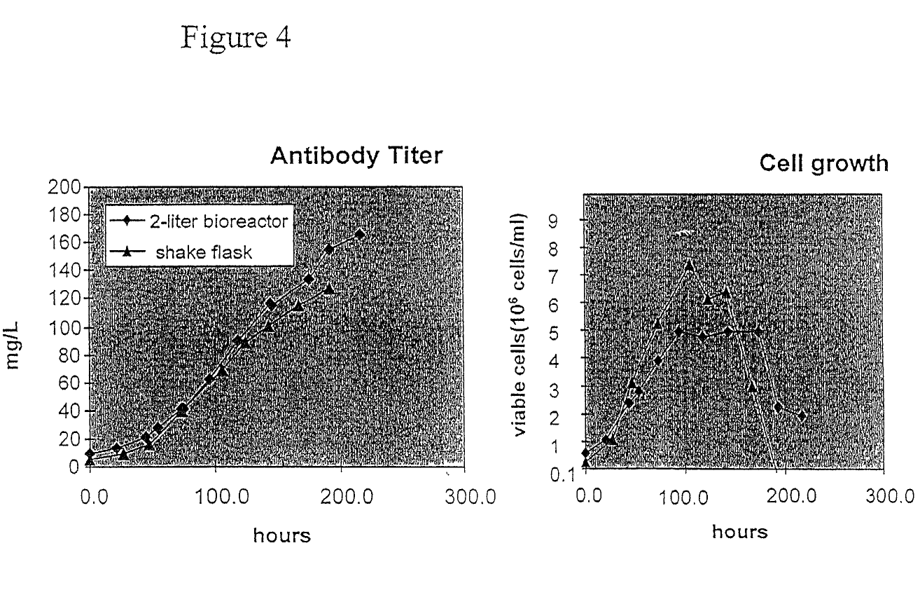

Transfections were carried out using lipofectamine (Life Technologies) according to the manufacturers' instructions. Cells were selected using hygromycin and G418 in CD-CHO medium (Life Technologies) and subclones were selected. This process took approximately 5 weeks. One subclone was scaled into a 2L bioreactor to perform final parameter optimization before being scaled into a 100L bioreactor. Production rates from the majority of transfectants expressing recombinant antibodies were typically approximately 5 pg/cell/day using this approach. Yields of one antibody in suspension culture reached approximately 200 mg/l. See FIG. 4. The inclusion of the UCOE in the two expression vectors co-transfected into CHO-S cells resulted in rapid isolation of a transfectant clone that could immediately be cultured in suspension in a defined medium.

### Example 3

### Low Levels of Gal-Gal Residues on CHO-K1 and CHO-S Host Cell-lines

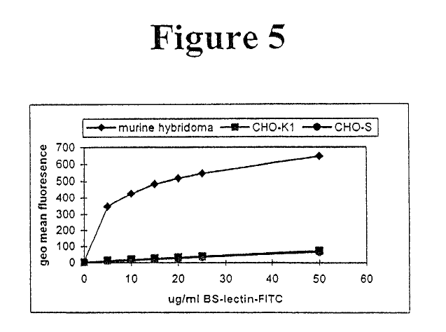

As discussed hereinabove, the presence of the Galα1→3Galβ1→4GlcNAc-R (Gal-Gal) carbohydrate residue on antibodies used as human therapeutics has been associated with rapid protein clearance from the serum. As a result, the ability to produce recombinant protein without this residue is advantageous. See, e.g., Borrebaeck et al., Immunology Today 14:477-479 (1993) and Kagawa et al., J. Biol. Chem. 263:17508-17515 (1988). Utilizing the FITC labeled IB₄ lectin and flow cytometry it was demonstrated that the Gal-Gal residue is not present on the surface of CHO-S cells. See FIG. 5; methodology disclosed in Cho et al., J. Biol. Chem. 272:13622-13628 (1997) and Gorelik et al., Cancer Res. 55:4185-4173 (1995). In this respect, CHO-S resembles the other widely used CHO line tested, CHO-K1. In contrast, the mouse hybridoma cell-line tested in this experiment showed high levels of cell-surface associated Gal-Gal carbohydrate. Mass spectroscopy of a purified recombinant protein produced in the cell-line demonstrated the absence of the Gal-Gal residue (data not shown).

### Example 4

### Bi-Directional UCOE Vectors for Improved Expression Levels of Multi-Subunit Recombinant Proteins

This Example discloses improved expression of recombinant antibody heavy and light protein chains on bi-directional UCOE vector systems.

The two Sfi I sites of pORT1 (Cobra Therapeutics) were changed to Mfe I sites by introduction of adapter molecules comprised of annealed oligos Mfe.F, 5′-AACAATTGGCGGC (SEQ ID NO: 10) and Mfe.R, 5′-GCCAATTGTTGCC (SEQ ID NO: 11). The HSV TK polyA site was then amplified from pVgRXR (Invitrogen) with primers TK.F, 5′ACGCGTCGACGGAAGGAGACAATACCGGAAG (SEQ ID NO: 12) and TK.R, 5′-CCGCTCGAGTTGGGGTGGGGAAAAGGAA (SEQ ID NO: 13), and the Sal I to Xho I fragment was inserted into the Sal I site. Following this, the murine PGK polyA site was amplified from male BALB/c genomic DNA (Clontech) using primers mPGK.F, 5′-CGGGATCCGCCTGAGAAAGGAAGTGAGCTG (SEQ ID NO: 14) and mPGK.R, 5′-GAAGATCTGGAGGAATGAGCTGGCCCTTA (SEQ ID NO: 15), and the BamH I to Bgl II fragment was cloned into the BamH I site. The Ase I to Sal I fragment of pcDNA3.1 containing the neo expression cassette was treated with T4 DNA polymerase, ligated to Spe I linkers (5′-GACTAGTC; SEQ ID NO: 16) and the Spe I fragment was then cloned into the Spe I site to give pORTneoF; or the EcoR I to Not I fragment of CET700 (Cobra Therapeutics) carrying the puromycin resistance cassette was treated with T4 DNA polymerase, ligated to Xba I linkers, and the Xba I fragment was cloned into the Xba I site to give pORTpuroF. The Hind III to BamH I murine CMV promoter fragment from pCMVEGFPN-1 (Cobra) was subcloned into the Hind III to BamH I sites of the Hybrid UCOE in BKS+ (Cobra). The human CMV promoter was then amplified from plasmid pIRESneo (Clontech) using primers hCMVF, 5′-CTCGAGTTATTAATAGTAATCAATTACGGGGTCAT (SEQ ID NO: 17) and hCMVR, 5′-GTCGACGATCTGACGGTTCACTAAACCAGCTCT (SEQ ID NO: 18) and the Xho I to Sal I fragment was cloned into the Sal I site. The BamH I to Sal I fragment was then cloned into the BamH I to Sal I sites of pORTneoF to give pBDUneo100, or into pORTpuroF to give pBDUpuro300. The two ATG codons upstream of the Sal I cloning site in the Hybrid UCOE in BKS+ were altered by site-directed mutagenesis, then the BamH I to Sal I fragment was cloned into the BamH I to Sal I sites of pORTneoF to give pBDUneo200, or into pORTpuroF to give pBDUpuro400.

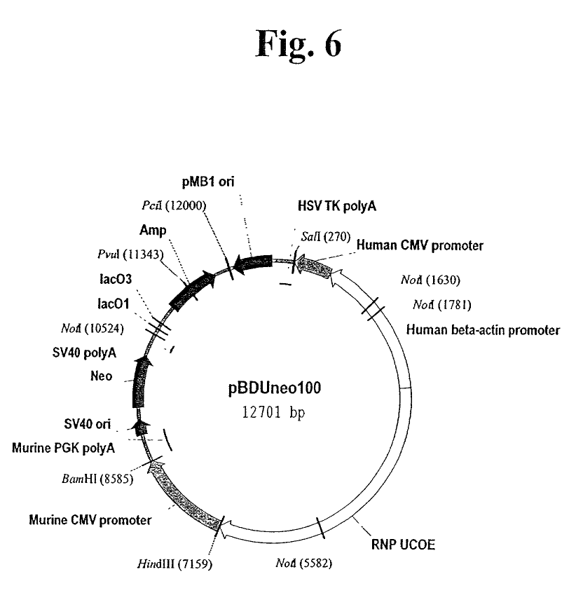

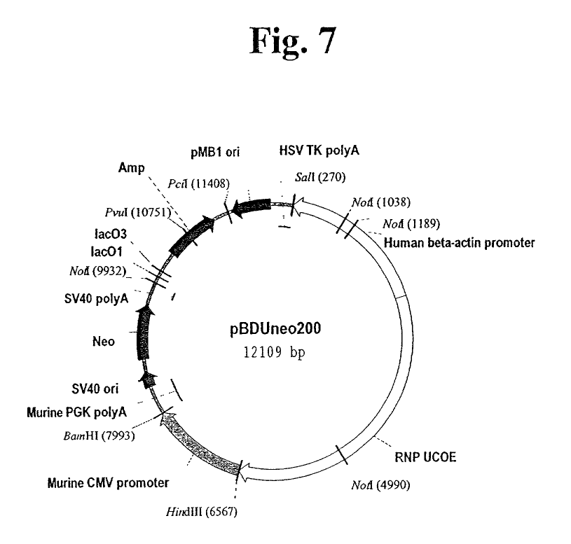

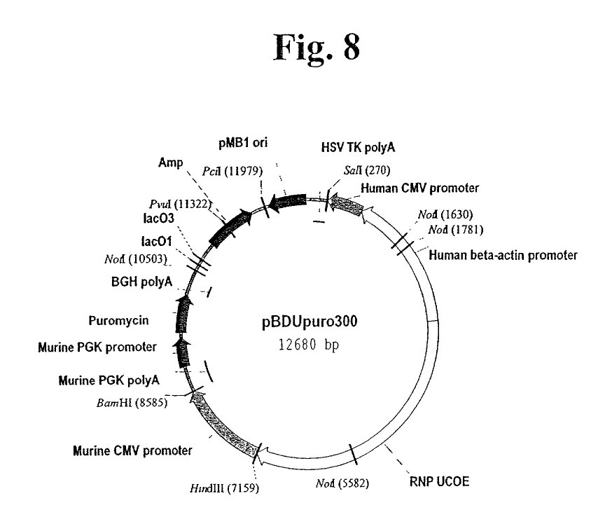

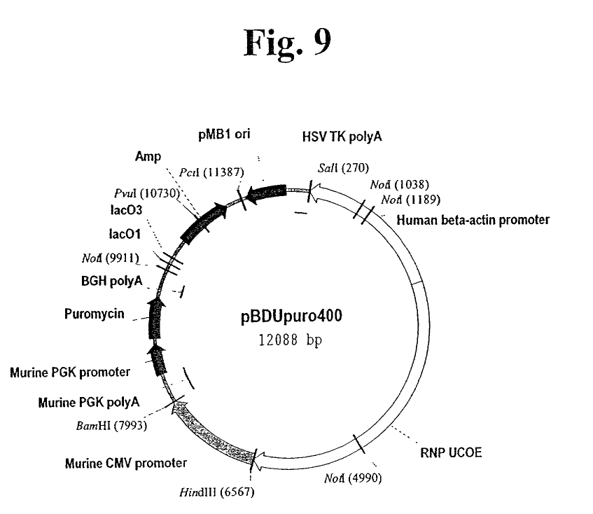

Human antibody light chains were cloned into either the BamH I or Sal I sites of all four bi-directional UCOE vectors (pBDUneo100, pBDUneo200, pBDUpuro300 and pBDUpuro400; FIGS. 6-9 and SEQ ID NOs: 1-4, respectively), followed by the heavy chain at the remaining BamH I or Sal I cloning site to give pBDUneo112, pBDUneo121, pBDUneo212, pBDUneo221, pBDUpuro112, pBDUpuro12l, pBDUpuro212 and pBDUpuro221.

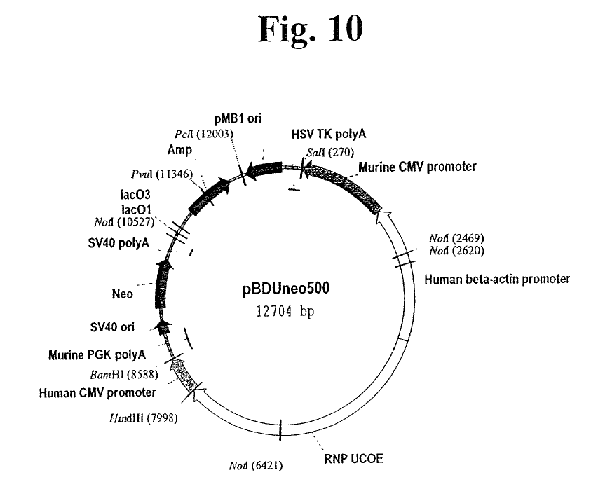

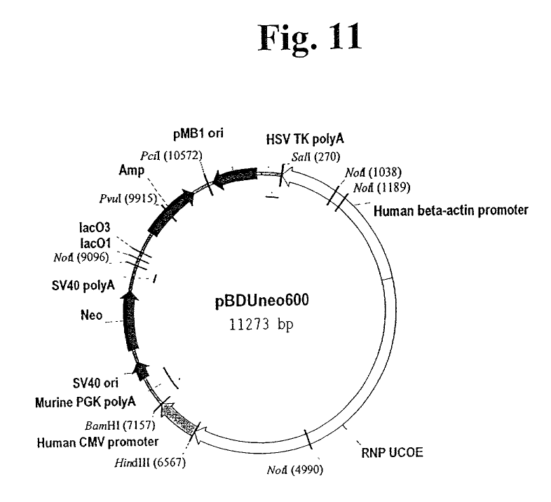

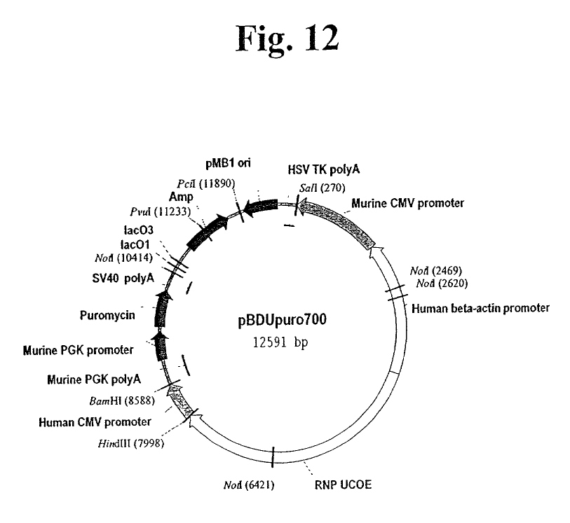

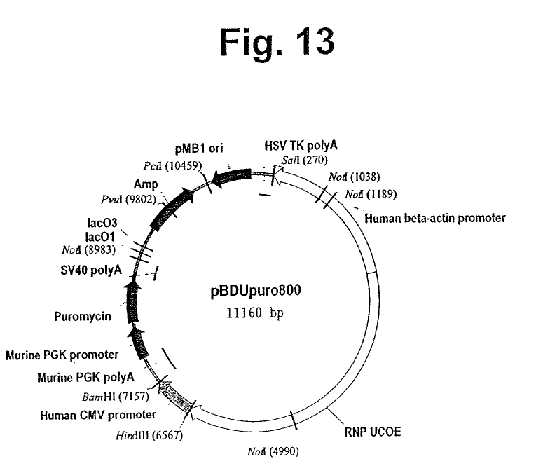

Additional bi-directional UCOE vectors suitable for co-expression of two or more recombinant proteins are disclosed in FIGS. 10-13 (SEQ ID NOs: 5-8) and are referred to as pBDUneo500, pBDUneo600, pBDUpuro700 and pBDUpuro800, respectively. These vectors may be employed, for example, to optimize the hybrid UCOE orientation for antibody expression, as well as to provide alternative promoter combinations for optimization.

Plasmid pORTpuroF was digested with XbaI (partial) and NsiI to remove the bovine growth hormone polyA site, then ligated to the SV40 early polyA site which was amplified with primers 14506, 5′-CCAATGCATAGGTTGGGCTTCGGGAATCGT (SEQ ID NO: 19) and 14507, 5′-GCTCTAGATCTCGACGGTATACAGACATGAT (SEQ ID NO: 20) followed by digestion with XbaI and NsiI, to give plasmid pORTpuroF2. The Hybrid UCOE vector containing the murine CMV promoter downstream of the human RNP UCOE and with the two mutated ATG codons between the actin promoter and the Sal I site, was digested with BamHI and HindIII to remove the murine CMV promoter, then ligated to the human CMV promoter that had been amplified with primers 14425, 5′-CCCAAGCTTATTAATAGTAATCAATTACGGGGTCAT (SEQ ID NO: 21) and 14426, 5′-CAAGGATCCGATCTGACGGTTCACTAAACCAGCTCT (SEQ ID NO: 22) followed by digestion with BamHI and HindIII. An adapter comprised of annealed oligos 14466, 5′-TCGAGTCGTTTAAACTCTAG (SEQ ID NO: 23) and 14465, 5′-TCGACTAGAGTTTAAACGAC (SEQ ID NO: 24) was then inserted at the SalI site, digested with PmeI and SalI, and ligated to the murine CMV promoter that had been amplified with primers 14435, 5′-GAATTCGAGCTCGCCCAACTCCGCCCGTTTTAT (SEQ ID NO: 25) and 14436, 5′-ATTTGTCGACTCTAGACCCGGGCTGCAGCGAGGAGCTCT (SEQ ID NO: 26) followed by digestion with SalI. The plasmid either with, or without, the murine CMV promoter was then digested with BamHI and SalI, and ligated to BamHI and SalI digested pORTneoF to give plasmids pBDUneo500 and pBDUneo600; or was ligated to BamHI and SalI digested plasmid pORTpuroF2 to give plasmids pBDUpuro700 and pBDUpuro800, respectively.

G418 or puromycin-resistant bi-directional UCOE vectors expressing antibody heavy and light chains were transfected into CHO-K1 or CHO-S cells using Lipofectamine or DMRIE-C (Invitrogen), respectively, following the manufacturer's instructions, and selected with 500 ug/ml G418 (neo vectors) or 12.5 ug/ml puromycin (puro vectors). Pools were selected and antibody production rates compared between the different constructs to determine the optimal promoter and selectable marker combination for antibody expression in CHO cells.

The results of expression studies in CHO-S suspensions cells are depicted in Table 2. These data demonstrated that vectors containing the light chain expressed from the murine CMV promoter gave the best antibody expression. Vectors containing puromycin or G418-resistance markers were used. Additionally, two bi-directional vectors, one containing a puromycin-resistance marker and one containing a G418-resistance marker, were co-transfected. Pools were selected, and antibody production rates determined. Separately, the G418 or puromycin-resistant transfecant pools displayed similar production rates, but the production rate of the co-transfected pool was significantly higher. This suggests that it may be possible to increase production rate by having two copies of the antibody expression vector, maintained with different selectable markers. Selecting pools with higher levels of puromycin (25-50 μg/ml versus 12.5 μg/ml) did not correlate with increased production.

Clonal lines were isolated from the puromycin-resistant pool carrying pBDUpuro421. Fifteen out of twenty-two clonal cell lines expressed measurable amounts of antibody. Initial production-rate determinations indicated that the cell lines had antibody secretion rates of up to 16 pg/cell/day (Table 3). Southern blot analysis identified at least one clone having a production rate of 13 pg/cell/day and has approximately a single copy of the vector DNA (clone S421.7). Clones from this pool were isolated with production rates of 3-18 pg/cell/day. Clones expressing approx. 5 pg/cell/day were used for initial fermentation experiments.

TABLE 2
Expression of hAb1 (IgG4) from bi-directional UCOE vectors

| | | | Production |
|---|---|---|---|
| | | | Rate |
| Vector | H3 Promoter | K1 Promoter | (pg/cell/day) |
| pBDUneo112 | murine CMV | human CMV | 0.3 |
| pBDUneo121 | human CMV | murine CMV | 1.5 |
| pBDUneo212 | murine CMV | human beta-actin | 0.06 |
| pBDUneo221 | human beta-actin | murine CMV | 1.3 |
| pBDUpuro312 | murine CMV | human CMV | 0.5 |
| pBDUpuro321 | human CMV | murine CMV | 1.4 |
| pBDUpuro412 | murine CMV | human beta-actin | 0.05 |
| pBDUpuro421 | human beta-actin | murine CMV | 2.3 |
| Cotransfection** | human CMV | human CMV | 0.7 |
| pBDUneo221 | human beta-actin | murine CMV | 1.3 |
| pBDUpuro421 | human beta-actin | murine CMV | 1 |
| pBDUneo221+ | human beta-actin | murine CMV | 5 |
| pBDUpuro421 | | | |

**Cotransfection was carried out previously using the same antibody genes each driven from 4 kb UCOE CMV vectors (hygromycin and neomycin selection)

TABLE 3
Expression of hAb1 in clonal CHO-S cell lines
transfected with pBDUpuro421

| | Production Rate |
|---|---|
| Puromycin^{R} Cell Line | (pg/cell/day) |
| S421.2 | 5.4 |
| S421.3 | 0.5 |
| S421.4 | 0.5 |
| S421.7 | 13.4 |
| S421.8 | 5.4 |
| S421.9 | 0.04 |
| S421.12 | 1.4 |
| S421.14 | 6.7 |
| S421.15 | 0.3 |
| S421.16 | 7.2 |
| S421.17 | 5 |
| S421.18 | 0.8 |
| S421.20 | 1.2 |
| S421.21 | 0.3 |
| S421.22 | 16 |

### Example 5

### Deletion Analysis of the RNP UCOE

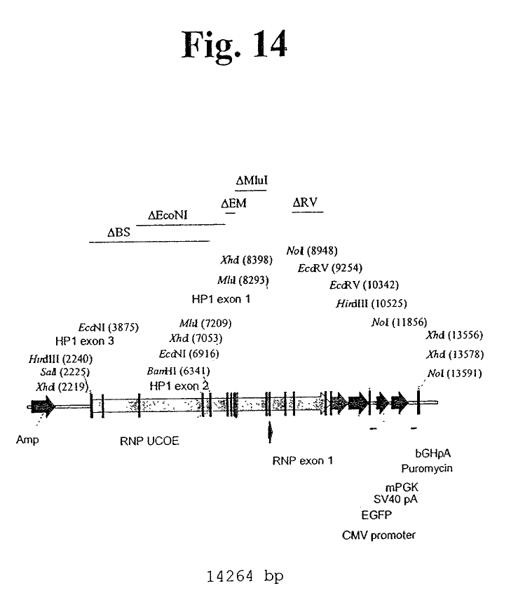

This Example discloses polynucleotide deletions within an RNP UCOE plasmid vector for improved expression of recombinant proteins. Briefly, a series of deletions within the 8 kb RNP UCOE were prepared to identify both important functional elements and regions that may be removed without affecting UCOE function. A green fluorescent protein gene (GFP) was cloned into plasmid CET720 (Cobra Therapeutics), and deletions were subsequently introduced into the UCOE region (FIG. 14). The first set of these deletions was transfected into CHO-S cells, and examined for the ability to express GFP. In a transient assay (two days post transfection), all of the plasmids were able to express GFP as determined by fluorescence microscopy. Stable pools carrying the different constructs were then selected, and GFP expression determined by FACS analysis. One month post-transfection, all of the deletions displayed both a higher percentage of positive cells than a control plasmid which did not contain the UCOE (>50% versus 10% without the UCOE), and a higher mean fluorescence for the positive population than the control vector that did not contain the UCOE (Table 4).

These data defined more precisely the region of the human RNP UCOE required for full activity and identified a shorter (approximately 7 kb) UCOE element with full activity. This new 7 kb UCOE element was defined by deletion ARV and extends from nucleotide 2225-9254 in FIG. 14.

TABLE 4
GFP expression from plasmids containing
deletions within the 8 kb RNP UCOE

| | | Percent | Mean Fluorescence of |
|---|---|---|---|
| Plasmid | Region Deleted | Positive | Positive Population |
| CET720GFP (8 kb | None | 68 | 516 |
| UCOE) | | | |
| CET700GFP (no | nt. 2225-10525 | 10 | 136 |
| UCOE) | | | |
| ΔBS (4 kb UCOE) | nt. 2225-6341 | 61 | 370 |
| ΔEcoNI | nt. 3875-6916 | 65 | 439 |
| ΔEX2 | nt. 6916-7053 | 53 | 384 |
| ΔEM | nt. 6916-7209 | 66 | 423 |
| ΔMX | nt. 7053-7209 | 66 | 464 |
| ΔMluI | nt. 7209-8293 | 58 | 448 |
| ΔRV | nt. 9254-10342 | 72 | 548 |

Vector CET720GFP (represented by SEQ ID NO: 9, which contains the 8 kb human RNP UCOE) was digested with EcoRV, MluI, EcoNI, or BamHI plus SalI, the ends were blunted with T4 DNA polymerase and religated to produce vectors deltaRV, delta MluI, deltaEcoNI and deltaBS, respectively. CET720 was digested with PflMI and blunted with T4 DNA polymerase, then cut with BamHI. The blunt to BamHI fragment was cloned into the EcoRV to BamHI sites of pBluescript II SK (+) to give pPB720. pPB720 was digested with EcoNI and MluI, MluI and XhoI (partial), or EcoNI and XhoI (partial), the ends were treated with T4 DNA polymerase and recircularized. The PshAI fragment from each of the resulting vectors was cloned into the PshAI sites of CET720GFP to give illustrative vectors deltaEM, deltaEX and deltaMX, respectively.

### Example 6

### Additional Deletion Analysis of the RNP UCOE

Previous examples have identified via deletion analysis that the UCOE regions from nucleotides 2225-6916 and 9254-10342 of vector CET720GFP (SEQ ID NO:9) can be removed without loss of UCOE activity (see Example 5 above). In this example, minimal regions of the 8 kb RNP UCOE that are important for its activity are further defined. Importantly, this analysis more precisely defined an illustrative 4.1 kb region of the human RNP UCOE that retains for full activity.

Briefly, fragments of the 8 kb RNP UCOE were blunted and ligated to HindIII linkers (New England Biolabs; Catalog Number S1098S), digested with HindIII and ligated to HindIII digested and calf-intestinal alkaline phosphatase-treated vector CET700GFP. Vectors were transfected into CHO-S cells using DMRIE-C (Invitrogen), where all constructs were capable of expressing GFP in a transient assay (data not shown). After 2 weeks in puromycin selection, the geometric mean fluorescence of the positive population was determined by FACS, and expressed as a percentage of the control (CET720GFP), the results of which are summarized in Table 5 below. Vector 700FRV, which contains a 4.1 kb MfeI to EcoRV fragment of the RNP UCOE, corresponding to nucleotide residues 5152-9254 of CET720GFP, retained full UCOE activity relative to the 8 kb UCOE region of nucleotide residues 2225-10525 of CET720GFP. Thus, this 4.1 kb UCOE fragment represents a new minimal UCOE element that retains activity at levels comparable to that for the full 8 kb UCOE element.

TABLE 5

| | | Percent |
|---|---|---|
| Plasmid | UCOE Region Present | of Control |
| CET720GFP (8 kb UCOE) | Nucleotides 2225-10525 | 100 |
| CET700GFP (no UCOE) | None | 10 |
| delta RV | Nucleotides 2225-9254 | 99 |
| | Nucleotides 10342-10525 | |
| 700HRV.R | Nucleotides 2240-9254 | 121 |
| 700FRV.R | Nucleotides 5152-9254 | 122 |
| 700BRV.R | Nucleotides 6341-9254 | 73 |

Activity was also determined for the three UCOE fragments contained within 700HRV.R, 700FRV.R and 700BRV.R, but with the UCOE fragments inserted in the opposite orientation, to give plasmids 700HRV.F, 700FRV.F and 700BRV.F, respectively. Again, all plasmids were capable of expressing GFP in a transient assay. After 3 weeks in puromycin selection, the geometric mean fluorescence of the positive population was determined by FACS, and expressed as a percentage of the control (CET720GFP), the results of which are summarized in Table 6 below. While lower levels of activity were observed for plasmids containing UCOE in the opposite orientation, all fragments nonetheless retained UCOE activity.

TABLE 6

| | | Percent |
|---|---|---|
| Plasmid | UCOE Region Present | of Control |
| CET720GFP (8 kb UCOE) | Nucleotides 2225-10525 | 100 |
| CET700GFP (no UCOE) | None | 6 |
| 700HRV.F | Nucleotides 2240-9254 | 59 |
| 700FRV.F | Nucleotides 5152-9254 | 43 |
| 700BRV.F | Nucleotides 6341-9254 | 30 |

### Example 7

### Preparation of Additional Illustrative Bi-Directional UCOE Vectors

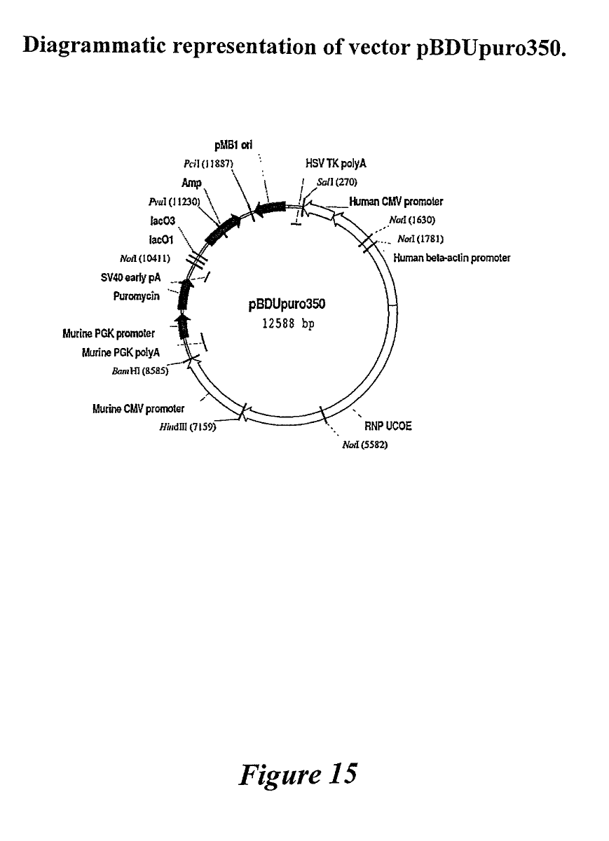

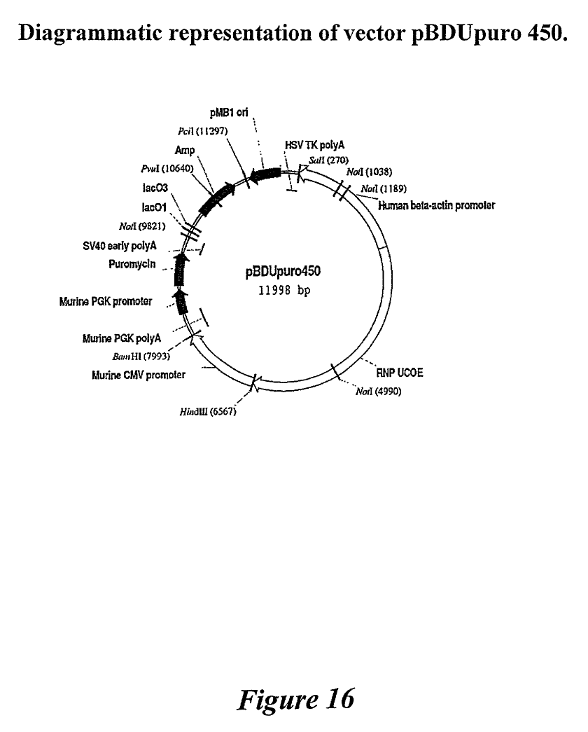

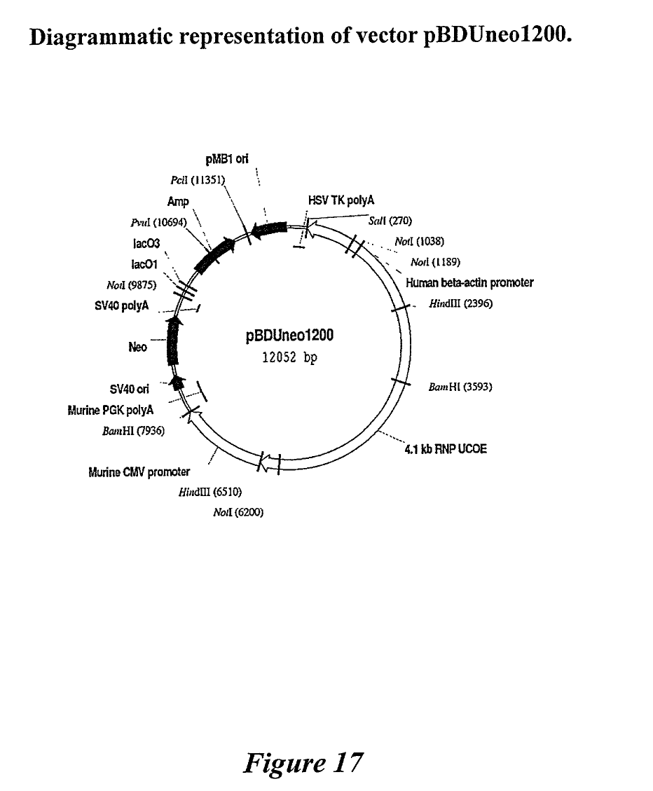

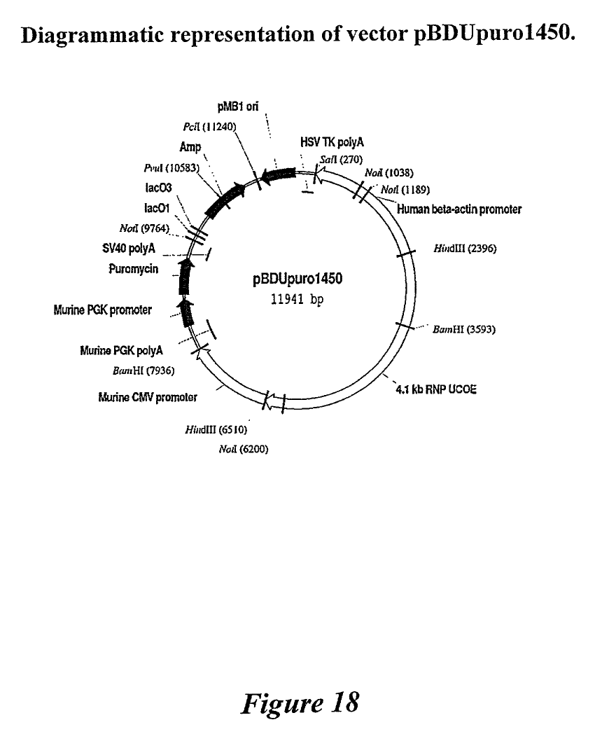

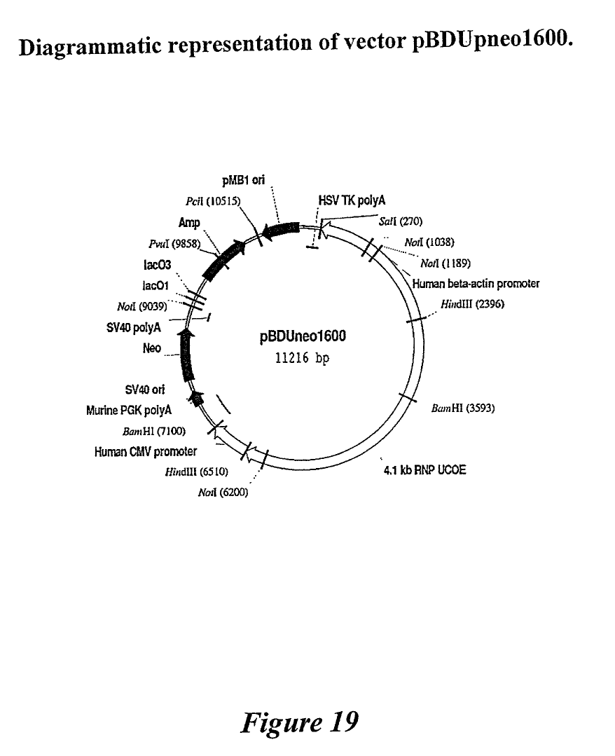

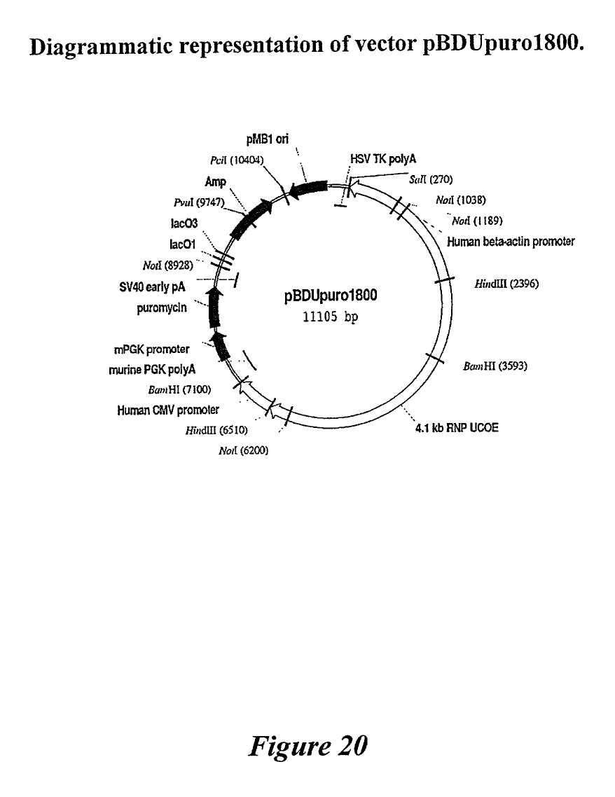

Previous examples have described the preparation and evaluation of numerous illustrative UCOE vectors. In this example, additional UCOE vectors were constructed. For example, vectors pBDUpuro350 (SEQ ID NO: 27) and pBDUpuro450 (SEQ ID NO: 28) were prepared so as to be equivalent to the previously described vectors pBDUpuro300 and pBDUpuro400, with the exception that the polyA site following the puromycin resistance gene was replaced with the SV40 polyA site (see also FIGS. 15 and 16). Several additional vectors will replace the 8 kb RNP UCOE element with the 4.1 kb MfeI-EcoRV fragment identified hereinabove by deletion analysis to retain full UCOE activity. To alter the polyA site of the puromycin resistance cassette of the pBDUpuro vector series, the SV40 polyA site was amplified from pBSneo.23 by polymerase chain reaction and the reaction product was digested with NsiI and XbaI and inserted into the NsiI to XbaI site of pORTpuroF to replace the BGH polyA site. This new vector, pORTpuroF′ was sequentially digested with BamHI and SalI, and cloned into the BamHI to SalI sites of HUCMV (hybrid UCOE with murine CMV promoter) to give plasmid pBDUpuro350 (SEQ ID NO: 27; see also FIG. 15), or cloned into the BamHI site of pUCOEact3 (hybrid UCOE with site directed mutagenesis of the ATG codons in the actin promoter) to give pBDUpuro450 (SEQ ID NO: 28; see also FIG. 16). Addditional UCOE vectors are constructed by inserting a HindIII site at the position of the KpnI site at the border between the human beta-actin and RNP UCOE fragments in plasmids pUCOEact3 and pUCOEact3hCMV. The 4 kb HindIII fragment carrying the RNP UCOE is then removed and replaced with the 4.1 kb RNP UCOE fragment from 700FRV.R. The SalI to BamHI (partial) fragments are then cloned into the SalI to BamHI sites of pORTneoF and pORTpuroF′ to give pBDUpuro1200 (SEQ ID NO: 29; see also FIG. 17), pBDUpuro1450 (SEQ ID NO: 30; see also FIG. 18), pBDUneo1600 (SEQ ID NO: 31; see also FIG. 19) and pBDUpuro1800 (SEQ ID NO: 32; see also FIG. 20).

### Example 8

### Evaluation of Vector Features Important for Bi-Directional UCOE Activity

1. Effect of Bi-directional UCOE Vector Copy Number on Antibody Production Rate in CHO-S Cells:

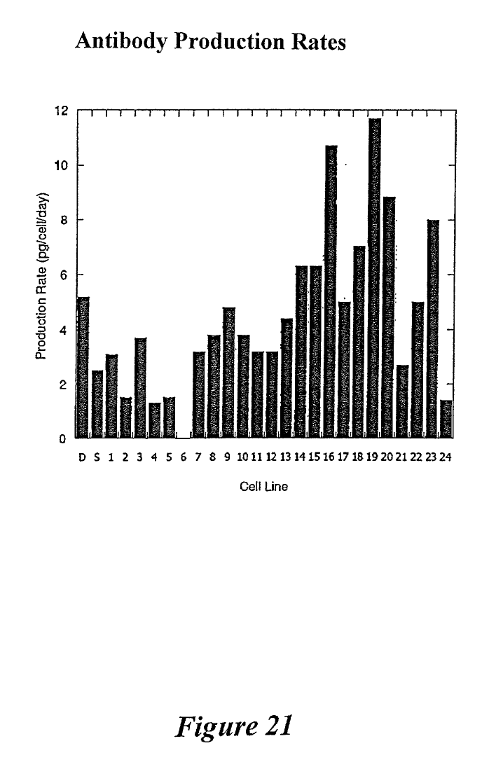

CHO-S cell line S421.7 have been shown to contain a single copy of vector pBDUpuro421, which expresses hAb1 (IgG4). To determine if additional vector copies could increase antibody expression levels, S421.7 was retransfected with vector pBDUneo221 that also expresses hAb1, but carries a different selectable marker (G418 resistance). Clonal cell lines were isolated and analyzed for production rate (FIG. 21). Many cell lines appear to have higher production rates than the parental line S421.7, indicating that additional vector copies can increase production. Initial copy number analysis indicated that cell lines S7.16, S7.20 and S7.23 contain 1-2 copies of vector pBDUneo221 (data not shown).

2. Effect of Hybrid UCOE Orientation and Promoter Choice on Antibody production in CHO-S Cells

Stable pools of CHO-S cells carrying various bi-directional UCOE vectors expressing hAb1 (IgG4) were analyzed to determine both the effect of the orientation of the hybrid UCOE relative to the antibody genes, and the effect of different promoters on antibody expression rates. CHO-S cells were transfected with a series of bi-directional UCOE vectors expressing hAb1 (IgG4), and stable pools were selected with either 12.5 μg/ml puromycin or 500 μg/ml G418. The location of the heavy chain (H) and the light chain (K) relative to the hybrid UCOE element (actin end versus RNP end) and the promoters used are shown in Table 7 below. Antibody production rates were measured by ELISA, and western blot analysis was performed to determine the distribution of light chain and heavy chain in the supernatant (supe) versus the cell lysate (lysate). The orientation of the hybrid UCOE showed only minor effects on antibody expression levels, however the choice of promoter combination resulted in some differences in production rates. The highest production rates were obtained in these experiments using illustrative vectors expressing the heavy chain from the human beta-actin promoter, and the light chain from either the murine CMV or human CMV promoters (e.g., pBDUpuro454 and pBDUpuro804).

TABLE 7

| | | | Heavy | Heavy | Kappa | Kappa | |
|---|---|---|---|---|---|---|---|
| | Actin | | Chain | Chain | Chain | Chain | Prod. Rate |
| Vector | End | RNP end | (supe) | (lysate) | (supe) | (lysate) | (pg/cell/day) |
| pBDUpuro352 | hCMV-K | mCMV-H | + | ++ | + | − | 0.159 |
| pBDUpuro354 | hCMV-H | mCMV-K | + | + | +++ | + | 0.256 |
| pBDUpuro452 | actin-K | mCMV-H | +/− | ++ | +/− | − | 0.0056 |
| pBDUpuro454 | actin-H | mCMV-K | ++ | + | +++ | ++ | 0.657 |
| pBDUpuro702 | hCMV-K | mCMV-H | ++ | ++ | ++ | + | 0.391 |
| pBDUpuro704 | hCMV-H | mCMV-K | ++ | ++ | ++ | +/− | 0.170 |
| pBDUpuro802 | actin-K | mCMV-H | +/− | +++ | +/− | − | 0.020 |
| pBDUpuro804 | actin-H | mCMV-K | +++ | +++ | +++ | ++ | 0.608 |

3. Transcription Versus Production Rates in CHO-S Cells

Clonal cell lines were isolated from the puromycin resistant pools carrying pBDUpuro452, pBDUpuro454 and pBDUpuro804. Approximately two thirds of clonal lines carrying pBDUpuro454 and pBDUpuro804 had measurable antibody production rates from 1 to 10 pg/cell/day, similar to previous results obtained with vector pBDUpuro421 (data not shown). TaqMan assays on genomic DNA samples suggested that clonal lines S452.3, S454.5 and S804.4 carried single copies of the bidirectional UCOE vectors pBDUpuro452, pBDUpuro454 and pBDUpuro804, respectively. Cell line S421.7, previously shown by Southern analysis to have a single copy of pBDUpuro421 (pBDUpuro400 with the heavy chain expressed from the human actin promoter, and the light chain from the murine CMV promoter) was included as a control. To study the correlation between production rate and transcription of the antibody chains, TaqMan RT-PCR assays were carried out on these lines, the results of which are summarized in Table 8 below. Both heavy and light chain RNA levels in line S452.3 were significantly lower than those observed in the control lines D6 and S421.7, that have been shown to express antibody well. However, lines S454.5 and S804.4 had RNA levels as well as production levels similar to the positive control lines. Together with western blot analysis (data not shown), these results indicate that the RNA levels of antibody heavy and light chains observed in these lines correlates with the production rates observed.

TABLE 8

| | Production Rate | Light Chain | Heavy Chain |
|---|---|---|---|
| Cell Line | (pg/cell/day) | (Ct) | (Ct) |
| CHO-S | 0 | 40 | 40 |
| D6 | 5.5 | 20.39 | 22.86 |
| S421.7 | 4.57 | 21.91 | 23.90 |
| S454.5 | 3.52 | 22.12 | 23.96 |
| S804.4 | 3.62 | 22.40 | 24.11 |
| S452.3 | 0.07 | 29.62 | 26.47 |

Ct, cycle number threshold; CHO-S, parental cell line; D6, clonal cell line carrying two pieces of a vector expressing the light chain and 4-8 copies of the heavy chain expressed from the hCMV promoter for hAb1; S421.7, clonal cell line carrying a single copy pBDupuro421; S454.5, clonal cell line carrying a single copy of pBDUpuro454; S804.4 clonal cell line carrying a single copy of pBDUpuro804; and S452.3, clonal cell line carrying a single copy of pBDUpuro452.

U.S. patents, U.S. patent application publications, U.S. patent applications, foreign patents, foreign patent applications and non-patent publications referred to in this specification and/or listed in the Application Data Sheet are incorporated herein by reference in their entirety.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed:

1. A composition for achieving high-level, large scale protein and/or polypeptide expression, said composition comprising:
(a) an immortalized host cell-line, capable of continuous growth in culture wherein said host cell-line is capable of growth in serum-free suspension culture, and
(b) a vector for sustained overexpression of a recombinant protein and/or polypeptide,
wherein said host cell-line is transfected with said vector.

2. The composition of claim 1 wherein said immortalized host cell-line has a doubling time of no more than 16 hours.

3. The composition of claim 2 wherein said doubling time is no more than 12 hours.

4. The composition of claim 1 having an efficiency of transfection of at least 70%.

5. The composition of claim 4 wherein said efficiency of transfection is at least 75%.

6. The composition of claim 4 wherein said efficiency of transfection is at least 85%.

7. The composition of claim 4 wherein said efficiency of transfection is at least 95%.

8. The composition of claim 1 wherein said host cell-line is susceptible to selection agents selected from the group consisting of: hygromycin, G418, and puromycin.

9. The composition of claim 1 wherein said host cell-line is characterized by the absence of gal-gal glycosylation of said recombinant protein and/or polypeptide.

10. The composition of claim 1 wherein said host cell-line is selected from the group consisting of CHO-S, 293-F, 293-H, COS-7L, D.Mel-2, Sf21, and Sf9.

11. The composition of claim 1 wherein said vector further comprises a property selected from the group consisting of (a) containing one or more elements that facilitate high-level, large-scale expression in the immortalized host cell-line and (b) resistance to repression of the recombinant protein and/or polypeptide.

12. The composition of claim 1 wherein said vector further comprises one or more universal chromatin opening elements (UCOEs).

13. The composition of claim 1 wherein said composition is characterized in being capable of achieving expression levels of at least 50 mg recombinant protein and/or polypeptide per liter of culture.

14. The composition of claim 13 wherein said composition is characterized in being capable of achieving expression levels of at least 100 mg recombinant protein and/or polypeptide per liter of culture.

15. The composition of claim 13 wherein said composition is characterized in being capable of achieving expression levels of at least 200 mg recombinant protein and/or polypeptide per liter of culture.

16. The composition of claim 1 wherein said composition is capable of scale-up to at least 100 liter scale and wherein said composition is capable of yields of at least 1 gram of protein and/or polypeptide.

17. The composition of claim 16 wherein said composition is capable of yields of at least 10 grams of protein and/or polypeptide.

18. The composition of claim 16 wherein said composition is capable of yields of at least 20 grams of protein and/or polypeptide.

19. A method for the high-level, large-scale production of a protein and/or polypeptide, said method comprising the steps of
(a) obtaining an immortilized host cell-line capable of growth in suspension;
(b) adapting said immortilized host cell-line for growth in serum-free medium;
(c) transfecting said serum-free growth adapted immortalized cell-line with a vector suitable for high-level expression of a recombinant protein and/or polypeptide.

20. The method of claim 19 wherein said immortalized host cell-line has a doubling time of no more than 16 hours.

21. The method of claim 20 wherein said doubling time is no more than 12 hours.

22. The method of claim 19 having an efficiency of transfection of at least 70%.

23. The method of claim 22 wherein said efficiency of transfection is at least 75%.

24. The method of claim 22 wherein said efficiency of transfection is at least 85%.

25. The method of claim 22 wherein said efficiency of transfection is at least 95%.

26. The method of claim 19 wherein said host cell-line is susceptible to selection agents selected from the group consisting of: hygromycin, G418, and puromycin.

27. The method of claim 19 wherein said host cell-line is characterized by the absence of gal-gal glycosylation of said recombinant of protein and/or polypeptide.

28. The method of claim 19 wherein said host cell-line is selected from the group consisting of CHO-S, 293-F, 293-H, COS-7L, D.Mel-2, Sf21, and Sf9.

29. The method of claim 19 wherein said vector further comprises a property selected from the group consisting of (a) containing one or more elements that facilitate high-level, large-scale expression in the immortalized host cell-line and (b) resistance to repression of the recombinant protein and/or polypeptide.

30. The method of claim 19 wherein said vector further comprises one or more universal chromatin opening elements (UCOEs).

31. The method of claim 19 wherein said method is characterized in being capable of achieving expression levels of at least 50 mg recombinant protein and/or polypeptide per liter of culture.

32. The method of claim 31 wherein said method is characterized in being capable of achieving expression levels of at least 100 mg recombinant protein and/or polypeptide per liter of culture.

33. The method of claim 31 wherein said method is characterized in being capable of achieving expression levels of at least 200 mg recombinant protein and/or polypeptide per liter of culture.

34. The method of claim 19 wherein said method is capable of scale-up to at least 100 liter scale and wherein said method is capable of yields of at least 1 gram of protein and/or polypeptide.

35. The method of claim 34 wherein said method is capable of yields of at least 10 grains of protein and/or polypeptide.

36. The method of claim 34 wherein said method is capable of yields of at least 20 grams of protein and/or polypeptide.

37. A bi-directional vector for high-level, large-scale expression, of a multisubunit protein and/or polypeptide, said composition comprising:
(a) at least one UCOE element; and
(b) a first transcriptional promoter; and
(c) a second transcriptional promoter;
wherein said UCOE element is operably linked to said first and said second transcriptional promoter and wherein said first transcriptional promoter is oriented in the opposite direction as said second transcriptional promoter

38. The bidirectional vector of claim 37 wherein said UCOE element is an RNP UCOE.

39. The bi-directional vector of claim 37 wherein said first transcriptional promoter is selected from the group consisting of a human CMV promoter, a murine CMV promoter and a human beta-actin promoter.

40. A composition for achieving high-level, large scale protein and/or polypeptide expression, said composition comprising:
(a) an immortalized host cell-line, capable of continuous growth in culture wherein said host cell-line is capable of growth in serum-free suspension culture, and
(b) the bi-directional vector of claim 37,
wherein said host cell-line is transfected with said vector.

41. A method for the high-level, large-scale production of a protein and/or polypeptide, said method comprising the steps of
(a) obtaining a host cell-line capable of continuous growth;
(b) adapting said host cell-line for growth in serum-free medium to create a cell-line capable of continuous growth in serum-free medium;
(c) transfecting said cell-line capable of continuous growth in serum-free medium with a vector of claim 37.

42. The method of claim 41 wherein said host cell-line capable of continuous growth is also capable of growth in suspension.

43. The method of claim 42 wherein said host cell-line capable of continuous growth in suspension is a CHO-S cell-line.

44. A vector for high-level, large scale expression, of a multisubunit protein and/or polypeptide, said composition comprising:
(a) at least one UCOE element; and
(b) a transcriptional promoter;
said vector further comprising one or more deletion within regions of the RNP UCOE selected from the group consisting of ΔBS, ΔEcoNI, ΔEM, ΔMluI, and ΔRV as depicted in Table 4 and FIG. 14.

45. The vector of claim 44 wherein said deletion is within the region of the RNP UCOE depicted by ΔBS in Table 4 and FIG. 14.

46. The vector of claim 44 wherein said deletion is at least 100 bp.

47. The vector of claim 44 wherein said deletion is at least 1,000 bp.

48. The vector of claim 44 wherein said deletion is at least 4,000 bp.


SEQUENCE LISTING (32 sequences)

SEQ ID NO: 1 - DNA, 12701 bp, Artificial Sequence
  Artificial Sequence containing human UCOE elements and vector sequence
acgttgtaaaacgacggccagtgaattgtaatacgactcactatagggcgaattgggtaccgggccccccctcgaggtcgagttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtcgacgatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactcgacggtatcatggtggcgaccggcatggtgagctgcgagaatagccgggcgcgctgtgagccgaagtcgcccccgccctggccacttccggcgcgccgagtccttaggccgccagggggcgccggcgcgcgcccagattggggacaaaggaagccgggccggccgcgttattaccataaaaggcaaacactggtcggaggcgtccccgcggcgcgcggcaggaagccaggccccaaccccctcccaaccgggcgccagccccgcctccgcccggttcaaacagcgaccgggtcgcgcgcgcgcacgcagcggccacaccctcgggcgccagcggctcgggcaggaagtggcgcaagcgcccgggccccagaacgcacgcgcgattagcgccattgagtcccagcgcgcacgcgcaattagcgccaattcccagcgcgcacgcagttagcgcccaaaggaccagcgcgcacgcgcatggcgccccagcccccaccgggcctgacgggggctacgccgcgcccaccgtgcgatccccattggcaagagcccggctcagacaaagaccccgccggttgcccccgccccgagagcggcacccccggagcgcgcccgcccgagcgcggcctcgcgcctgcgaactggcgtggggtgtcccccatctccggaggcccaggggcttctcccgcgccccccacggcggtccggttccgccccatgcgccccccgctgcggcccagacggcggctctgcacgggcgaagggccgcggccgcatgccccggtcggctggccgggcttacctggcggcgggtgtggacgggcggcggatcggcaaaggcgaggctctgtgctcgcgggcggacgcggtctcggcggtggtggcgcgtcgcgccgctgggttttatagggcgccgccgcggccgctcgagccataaaaggcaactttcggaacggcgcacgctgattggccccgcgccgctcactcaccggcttcgccgcacagtgcagcatttttttaccccctctcccctccttttgcgaaaaaaaaaaagagcgagagcgagattgaggaagaggaggagggagagttttggcgttggccgccttggggtgctgggcccgggggctgggggcgcgcgccgtggcccccgcgccccacgctgggcagtgcccggttcggccccgcatggccaggcctgcccccggcctgcccgtctctcgggccccccacccaccgcgggacatcctaggtgtggacatctcttgggcactgagcgcccaggtggggtgggccagggtctgcacgggtgccagggccctgggttctgtacgctcctgcagaaggagctcttggagggcatggagtggccaggcagtcactcccccttgccgacttcagagcaactgccctgaaagcagggcctgaggacctctggctgtggggctcagctagctaaatgtgctgggtgggtcactagggagagacctgggcttgagaggtagagtgtggtgttgggggagtcaggtggcttgcggccattagagtcgcaggaccacactccccaggacagggcaggggccagcggtccagtggctggaggtggcccgtgatgaaggctacaaacctacccagccgcagccctgggaaggaagtgggctctacagggcagggcaccttttaccctggagctgcctgcttttgagggtaacagtcacgcccagccaagaccaggcctggggcgttagtgggtgacctaggcactgcggggcgggggggctgggtctacacagcctgggtctgggcccaccgtccgttgtatgtctgctatgcgcagccacagctgaactgccctcccagaccatctggaggccgctgggggactctggggaccaagactccatgtgccacagaggattgggggcggggcggtgctaggaactcaaagccagcctgggaagaccctgtccttgtcaccctttcttgccttgggtctgtccactgagtagcacacaagaccgggtgggcagggtccgttctgctccgggaatcacagactgtgtgtacccaggtggtgggcatgcagcgatcagtggcgtgggaccacagagggggcccgcggtacctaaaacagcttcacatggcttaaaataggggaccaatgtcttttccaatctaagtcccatttataataaagtccatgttccatttttaaaggacaatcctttcggtttaaaaccaggcacgattacccaaacaactcacaacggtaaagcactgtgaatcttctctgttctgcaatcccaacttggtttctgctcagaaaccctccctctttccaatcggtaattaaataacaaaaggaaaaaacttaagatgcttcaaccccgtttcgtgacactttgaaaaaagaatcacctcttgcaaacacccgctcccgacccccgccgctgaagcccggcgtccagaggcctaagcgcgggtgcccgcccccacccgggagcgcgggcctcgtggtcagcgcatccgcggggagaaacaaaggccgcggcacgggggctcaagggcactgcgccacaccgcacgcgcctacccccgcgcggccacgttaactggcggtcgccgcagcctcgggacagccggccgcgcgccgccaggctcgcggacgcgggaccacgcgccgccctccgggaggcccaagtctcgacccagccccgcgtggcgctgggggagggggcgcctccgccggaacgcgggtgggggaggggagggggaaatgcgctttgtctcgaaatggggcaaccgtcgccacagctccctaccccctcgagggcagagcagtccccccactaactaccgggctggccgcgcgccaggccagccgcgaggccaccgcccgaccctccactccttcccgcagctcccggcgcggggtccggcgagaaggggaggggaggggagcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcctcggcgagcgtttcggcgggcagcaggtcctcgtgagcgaggctgcggagcttcccctccccctctctcccgggaaccgatttggcggccgccattttcatggctcgccttcctctcagcgttttccttataactcttttattttcttagtgtgctttctctatcaagaagtagaagtggttaactattttttttttcttctcgggctgttttcatatcgtttcgaggtggatttggagtgttttgtgagcttggatctttagagtcctgcgcacctcattaaaggcgctcagccttcccctcgatgaaatggcgccattgcgttcggaagccacaccgaagagcggggagggggggtgctccgggtttgcgggcccggtttcagagaagatatcaccacccagggcgtcgggccgggttcaatgcgagccgtaggacaaagaaaccattttatgtttttcctgtcttttttttcctttgagtaacggttttatctgggtctgcagtcagtaaaacgacagatgaaccgcggcaaaataaacataaattggaagccatcggccacgaggggcagggacgaaggtggttttctgggcgggggagggatattcgcgtcagaatcctttactgttcttaaggattccgtttaagttgtagagctgactcattttaagtaatgttgttactgagaagtttaacccttacgggacagatccatggacctttatagatgattacgaggaaagtgaaataacgattttgtccttagttatacttcgattaaaacatggcttcagaggctccttcctgtaatgcgtatggattgatgtgcaaaactgttttgggcctgggccgctctgtatttgaactttgttacttttctcattttgtttgcaatcttggttgaacattacattgataagcataaggtctcaagcgaagggggtctacctggttatttttctttgaccctaagcacgtttataaaataacattgtttaaaatcgatagtggacatcgggtaagtttggataaattgtgaggtaagtaatgagtttttgctttttgttagtgatttgtaaaacttgttataaatgtacattatccgtaatttcagtttagagataacctatgtgctgacgacaattaagaataaaaactagctgaaaaaatgaaaataactatcgtgacaagtaaccatttcaaaagactgctttgtgtctcataggagctagtttgatcatttcagttaattttttctttaatttttacgagtcatgaaaactacaggaaaaaaaatctgaactgggttttaccactactttttaggagttgggagcatgcgaatggagggagagctccgtagaactgggatgagagcagcaattaatgctgcttgctaggaacaaaaaataattgattgaaaattacgtgtgactttttagtttgcattatgcgtttgtagcagttggtcctggatatcactttctctcgtttgaggttttttaacctagttaacttttaagacaggtttccttaacattcataagtgcccagaatacagctgtgtagtacagcatataaagatttcagctctgaggtttttcctattgacttggaaaattgttttgtgcctgtcgcttgccacatggccaatcaagtaagcttcgaattcgagctcgcccaactccgcccgttttatgactagaaccaatagtttttaatgccaaatgcactgaaatcccctaatttgcaaagccaaacgccccctatgtgagtaatacggggactttttacccaatttcccaagcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcagcccgggtctagaggatccgcctgagaaaggaagtgagctgtaaaggctgagctctctctctgacgtatgtagcctctggttagcttcgtcactcactgttcttgactcagcatggcaatctgatgaaatcccagctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctccagatccactagtaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcstggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgagactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagggggtacctaggccgggcaacaattggcggccggccgcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccctaggccgggcaacaattggcggccggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactc

SEQ ID NO: 2 - DNA, 12109 bp, Artificial Sequence
  Artificial Sequence containing human UCOE elements and vector sequence
acgttgtaaaacgacggccagtgaattgtaatacgactcactatagggcgaattgggtaccgggccccccctcgaggtcgagttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtcgacggtatcaaggtggcgaccggaatggtgagctgcgagaatagccgggcgcgctgtgagccgaagtcgcccccgccctggccacttccggcgcgccgagtccttaggccgccagggggcgccggcgcgcgcccagattggggacaaaggaagccgggccggccgcgttattaccataaaaggcaaacactggtcggaggcgtccccgcggcgcgcggcaggaagccaggccccaaccccctcccaaccgggcgccagccccgcctccgcccggttcaaacagcgaccgggtcgcgcgcgcgcacgcagcggccacaccctcgggcgccagcggctcgggcaggaagtggcgcaagcgcccgggccccagaacgcacgcgcgattagcgccattgagtcccagcgcgcacgcgcaattagcgccaattcccagcgcgcacgcagttagcgcccaaaggaccagcgcgcacgcgcatggcgccccagcccccaccgggcctgacgggggctacgccgcgcccaccgtgcgatccccattggcaagagcccggctcagacaaagaccccgccggttgcccccgccccgagagcggcacccccggagcgcgcccgcccgagcgcggcctcgcgcctgcgaactggcgtggggtgtcccccatctccggaggcccaggggcttctcccgcgccccccacggcggtccggttccgccccatgcgccccccgctgcggcccagacggcggctctgcacgggcgaagggccgcggccgcatgccccggtcggctggccgggcttacctggcggcgggtgtggacgggcggcggatcggcaaaggcgaggctctgtgctcgcgggcggacgcggtctcggcggtggtggcgcgtcgcgccgctgggttttatagggcgccgccgcggccgctcgagccataaaaggcaactttcggaacggcgcacgctgattggccccgcgccgctcactcaccggcttcgccgcacagtgcagcatttttttaccccctctcccctccttttgcgaaaaaaaaaaagagcgagagcgagattgaggaagaggaggagggagagttttggcgttggccgccttggggtgctgggcccgggggctgggggcgcgcgccgtggcccccgcgccccacgctgggcagtgcccggttcggccccgcatggccaggcctgcccccggcctgcccgtctctcgggccccccacccaccgcgggacatcctaggtgtggacatctcttgggcactgagcgcccaggtggggtgggccagggtctgcacgggtgccagggccctgggttctgtacgctcctgcagaaggagctcttggagggcatggagtggccaggcagtcactcccccttgccgacttcagagcaactgccctgaaagcagggcctgaggacctctggctgtggggctcagctagctaaatgtgctgggtgggtcactagggagagacctgggcttgagaggtagagtgtggtgttgggggagtcaggtggcttgcggccattagagtcgcaggaccacactccccaggacagggcaggggccagcggtccagtggctggaggtggcccgtgatgaaggctacaaacctacccagccgcagccctgggaaggaagtgggctctacagggcagggcaccttttaccctggagctgcctgcttttgagggtaacagtcacgcccagccaagaccaggcctggggcgttagtgggtgacctaggcactgcggggcgggggggctgggtctacacagcctgggtctgggcccaccgtccgttgtatgtctgctatgcgcagccacagctgaactgccctcccagaccatctggaggccgctgggggactctggggaccaagactccatgtgccacagaggattgggggcggggcggtgctaggaactcaaagccagcctgggaagaccctgtccttgtcaccctttcttgccttgggtctgtccactgagtagcacacaagaccgggtgggcagggtccgttctgctccgggaatcacagactgtgtgtacccaggtggtgggcatgcagcgatcagtggcgtgggaccacagagggggcccgcggtacctaaaacagcttcacatggcttaaaataggggaccaatgtcttttccaatctaagtcccatttataataaagtccatgttccatttttaaaggacaatcctttcggtttaaaaccaggcacgattacccaaacaactcacaacggtaaagcactgtgaatcttctctgttctgcaatcccaacttggtttctgctcagaaaccctccctctttccaatcggtaattaaataacaaaaggaaaaaacttaagatgcttcaaccccgtttcgtgacactttgaaaaaagaatcacctcttgcaaacacccgctcccgacccccgccgctgaagcccggcgtccagaggcctaagcgcgggtgcccgcccccacccgggagcgcgggcctcgtggtcagcgcatccgcggggagaaacaaaggccgcggcacgggggctcaagggcactgcgccacaccgcacgcgcctacccccgcgcggccacgttaactggcggtcgccgcagcctcgggacagccggccgcgcgccgccaggctcgcggacgcgggaccacgcgccgccctccgggaggcccaagtctcgacccagccccgcgtggcgctgggggagggggcgcctccgccggaacgcgggtgggggaggggagggggaaatgcgctttgtctcgaaatggggcaaccgtcgccacagctccctaccccctcgagggcagagcagtccccccactaactaccgggctggccgcgcgccaggccagccgcgaggccaccgcccgaccctccactccttcccgcagctcccggcgcggggtccggcgagaaggggaggggaggggagcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcctcggcgagcgtttcggcgggcagcaggtcctcgtgagcgaggctgcggagcttcccctccccctctctcccgggaaccgatttggcggccgccattttcatggctcgccttcctctcagcgttttccttataactcttttattttcttagtgtgctttctctatcaagaagtagaagtggttaactattttttttttcttctcgggctgttttcatatcgtttcgaggtggatttggagtgttttgtgagcttggatctttagagtcctgcgcacctcattaaaggcgctcagccttcccctcgatgaaatggcgccattgcgttcggaagccacaccgaagagcggggagggggggtgctccgggtttgcgggcccggtttcagagaagatatcaccacccagggcgtcgggccgggttcaatgcgagccgtaggacaaagaaaccattttatgtttttcctgtcttttttttcctttgagtaacggttttatctgggtctgcagtcagtaaaacgacagatgaaccgcggcaaaataaacataaattggaagccatcggccacgaggggcagggacgaaggtggttttctgggcgggggagggatattcgcgtcagaatcctttactgttcttaaggattccgtttaagttgtagagctgactcattttaagtaatgttgttactgagaagtttaacccttacgggacagatccatggacctttatagatgattacgaggaaagtgaaataacgattttgtccttagttatacttcgattaaaacatggcttcagaggctccttcctgtaatgcgtatggattgatgtgcaaaactgttttgggcctgggccgctctgtatttgaactttgttacttttctcattttgtttgcaatcttggttgaacattacattgataagcataaggtctcaagcgaagggggtctacctggttatttttctttgaccctaagcacgtttataaaataacattgtttaaaatcgatagtggacatcgggtaagtttggataaattgtgaggtaagtaatgagtttttgctttttgttagtgatttgtaaaacttgttataaatgtacattatccgtaatttcagtttagagataacctatgtgctgacgacaattaagaataaaaactagctgaaaaaatgaaaataactatcgtgacaagtaaccatttcaaaagactgctttgtgtctcataggagctagtttgatcatttcagttaattttttctttaatttttacgagtcatgaaaactacaggaaaaaaaatctgaactgggttttaccactactttttaggagttgggagcatgcgaatggagggagagctccgtagaactgggatgagagcagcaattaatgctgcttgctaggaacaaaaaataattgattgaaaattacgtgtgactttttagtttgcattatgcgtttgtagcagttggtcctggatatcactttctctcgtttgaggttttttaacctagttaacttttaagacaggtttccttaacattcataagtgcccagaatacagctgtgtagtacagcatataaagatttcagctctgaggtttttcctattgacttggaaaattgttttgtgcctgtcgcttgccacatggccaatcaagtaagcttcgaattcgagctcgcccaactccgcccgttttatgactagaaccaatagtttttaatgccaaatgcactgaaatcccctaatttgcaaagccaaacgccccctatgtgagtaatacggggactttttacccaatttcccaagcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcagcccgggtctagaggatccgcctgagaaaggaagtgagctgtaaaggctgagctctctctctgacgtatgtagcctctggttagcttcgtcactcactgttcttgactcagcatggcaatctgatgaaatcccagctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctccagatccactagtaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcstggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgagactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagggggtacctaggccgggcaacaattggcggccggccgcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccctaggccgggcaacaattggcggccggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactc

SEQ ID NO: 3 - DNA, 12680 bp, Artificial Sequence
  Artificial Sequence containing human UCOE elements and vector sequence
acgttgtaaaacgacggccagtgaattgtaatacgactcactatagggcgaattgggtaccgggccccccctcgaggtcgagttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtcgacgatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactcgacggtatcatggtggcgaccggcatggtgagctgcgagaatagccgggcgcgctgtgagccgaagtcgcccccgccctggccacttccggcgcgccgagtccttaggccgccagggggcgccggcgcgcgcccagattggggacaaaggaagccgggccggccgcgttattaccataaaaggcaaacactggtcggaggcgtccccgcggcgcgcggcaggaagccaggccccaaccccctcccaaccgggcgccagccccgcctccgcccggttcaaacagcgaccgggtcgcgcgcgcgcacgcagcggccacaccctcgggcgccagcggctcgggcaggaagtggcgcaagcgcccgggccccagaacgcacgcgcgattagcgccattgagtcccagcgcgcacgcgcaattagcgccaattcccagcgcgcacgcagttagcgcccaaaggaccagcgcgcacgcgcatggcgccccagcccccaccgggcctgacgggggctacgccgcgcccaccgtgcgatccccattggcaagagcccggctcagacaaagaccccgccggttgcccccgccccgagagcggcacccccggagcgcgcccgcccgagcgcggcctcgcgcctgcgaactggcgtggggtgtcccccatctccggaggcccaggggcttctcccgcgccccccacggcggtccggttccgccccatgcgccccccgctgcggcccagacggcggctctgcacgggcgaagggccgcggccgcatgccccggtcggctggccgggcttacctggcggcgggtgtggacgggcggcggatcggcaaaggcgaggctctgtgctcgcgggcggacgcggtctcggcggtggtggcgcgtcgcgccgctgggttttatagggcgccgccgcggccgctcgagccataaaaggcaactttcggaacggcgcacgctgattggccccgcgccgctcactcaccggcttcgccgcacagtgcagcatttttttaccccctctcccctccttttgcgaaaaaaaaaaagagcgagagcgagattgaggaagaggaggagggagagttttggcgttggccgccttggggtgctgggcccgggggctgggggcgcgcgccgtggcccccgcgccccacgctgggcagtgcccggttcggccccgcatggccaggcctgcccccggcctgcccgtctctcgggccccccacccaccgcgggacatcctaggtgtggacatctcttgggcactgagcgcccaggtggggtgggccagggtctgcacgggtgccagggccctgggttctgtacgctcctgcagaaggagctcttggagggcatggagtggccaggcagtcactcccccttgccgacttcagagcaactgccctgaaagcagggcctgaggacctctggctgtggggctcagctagctaaatgtgctgggtgggtcactagggagagacctgggcttgagaggtagagtgtggtgttgggggagtcaggtggcttgcggccattagagtcgcaggaccacactccccaggacagggcaggggccagcggtccagtggctggaggtggcccgtgatgaaggctacaaacctacccagccgcagccctgggaaggaagtgggctctacagggcagggcaccttttaccctggagctgcctgcttttgagggtaacagtcacgcccagccaagaccaggcctggggcgttagtgggtgacctaggcactgcggggcgggggggctgggtctacacagcctgggtctgggcccaccgtccgttgtatgtctgctatgcgcagccacagctgaactgccctcccagaccatctggaggccgctgggggactctggggaccaagactccatgtgccacagaggattgggggcggggcggtgctaggaactcaaagccagcctgggaagaccctgtccttgtcaccctttcttgccttgggtctgtccactgagtagcacacaagaccgggtgggcagggtccgttctgctccgggaatcacagactgtgtgtacccaggtggtgggcatgcagcgatcagtggcgtgggaccacagagggggcccgcggtacctaaaacagcttcacatggcttaaaataggggaccaatgtcttttccaatctaagtcccatttataataaagtccatgttccatttttaaaggacaatcctttcggtttaaaaccaggcacgattacccaaacaactcacaacggtaaagcactgtgaatcttctctgttctgcaatcccaacttggtttctgctcagaaaccctccctctttccaatcggtaattaaataacaaaaggaaaaaacttaagatgcttcaaccccgtttcgtgacactttgaaaaaagaatcacctcttgcaaacacccgctcccgacccccgccgctgaagcccggcgtccagaggcctaagcgcgggtgcccgcccccacccgggagcgcgggcctcgtggtcagcgcatccgcggggagaaacaaaggccgcggcacgggggctcaagggcactgcgccacaccgcacgcgcctacccccgcgcggccacgttaactggcggtcgccgcagcctcgggacagccggccgcgcgccgccaggctcgcggacgcgggaccacgcgccgccctccgggaggcccaagtctcgacccagccccgcgtggcgctgggggagggggcgcctccgccggaacgcgggtgggggaggggagggggaaatgcgctttgtctcgaaatggggcaaccgtcgccacagctccctaccccctcgagggcagagcagtccccccactaactaccgggctggccgcgcgccaggccagccgcgaggccaccgcccgaccctccactccttcccgcagctcccggcgcggggtccggcgagaaggggaggggaggggagcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcctcggcgagcgtttcggcgggcagcaggtcctcgtgagcgaggctgcggagcttcccctccccctctctcccgggaaccgatttggcggccgccattttcatggctcgccttcctctcagcgttttccttataactcttttattttcttagtgtgctttctctatcaagaagtagaagtggttaactattttttttttcttctcgggctgttttcatatcgtttcgaggtggatttggagtgttttgtgagcttggatctttagagtcctgcgcacctcattaaaggcgctcagccttcccctcgatgaaatggcgccattgcgttcggaagccacaccgaagagcggggagggggggtgctccgggtttgcgggcccggtttcagagaagatatcaccacccagggcgtcgggccgggttcaatgcgagccgtaggacaaagaaaccattttatgtttttcctgtcttttttttcctttgagtaacggttttatctgggtctgcagtcagtaaaacgacagatgaaccgcggcaaaataaacataaattggaagccatcggccacgaggggcagggacgaaggtggttttctgggcgggggagggatattcgcgtcagaatcctttactgttcttaaggattccgtttaagttgtagagctgactcattttaagtaatgttgttactgagaagtttaacccttacgggacagatccatggacctttatagatgattacgaggaaagtgaaataacgattttgtccttagttatacttcgattaaaacatggcttcagaggctccttcctgtaatgcgtatggattgatgtgcaaaactgttttgggcctgggccgctctgtatttgaactttgttacttttctcattttgtttgcaatcttggttgaacattacattgataagcataaggtctcaagcgaagggggtctacctggttatttttctttgaccctaagcacgtttataaaataacattgtttaaaatcgatagtggacatcgggtaagtttggataaattgtgaggtaagtaatgagtttttgctttttgttagtgatttgtaaaacttgttataaatgtacattatccgtaatttcagtttagagataacctatgtgctgacgacaattaagaataaaaactagctgaaaaaatgaaaataactatcgtgacaagtaaccatttcaaaagactgctttgtgtctcataggagctagtttgatcatttcagttaattttttctttaatttttacgagtcatgaaaactacaggaaaaaaaatctgaactgggttttaccactactttttaggagttgggagcatgcgaatggagggagagctccgtagaactgggatgagagcagcaattaatgctgcttgctaggaacaaaaaataattgattgaaaattacgtgtgactttttagtttgcattatgcgtttgtagcagttggtcctggatatcactttctctcgtttgaggttttttaacctagttaacttttaagacaggtttccttaacattcataagtgcccagaatacagctgtgtagtacagcatataaagatttcagctctgaggtttttcctattgacttggaaaattgttttgtgcctgtcgcttgccacatggccaatcaagtaagcttcgaattcgagctcgcccaactccgcccgttttatgactagaaccaatagtttttaatgccaaatgcactgaaatcccctaatttgcaaagccaaacgccccctatgtgagtaatacggggactttttacccaatttcccaagcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcagcccgggtctagaggatccgcctgagaaaggaagtgagctgtaaaggctgagctctctctctgacgtatgtagcctctggttagcttcgtcactcactgttcttgactcagcatggcaatctgatgaaatcccagctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctccagatccactagttctagagcaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaccagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgtagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaaggggggggattgggragacaatagcaggcatgctgggggggcggtgggggctatggcttctgaggcggaaagaaccagctggggctcgagatccactagttctagcctcgaggctagagcggcctgctctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagggggtacctaggccgggcaacaattggcggccggccgcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccctaggccgggcaacaattggcggccggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactc

SEQ ID NO: 4 - DNA, 12088 bp, Artificial Sequence
  Artificial Sequence containing human UCOE elements and vector sequence
acgttgtaaaacgacggccagtgaattgtaatacgactcactatagggcgaattgggtaccgggccccccctcgaggtcgagttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtcgacggtatcaaggtggcgaccggaatggtgagctgcgagaatagccgggcgcgctgtgagccgaagtcgcccccgccctggccacttccggcgcgccgagtccttaggccgccagggggcgccggcgcgcgcccagattggggacaaaggaagccgggccggccgcgttattaccataaaaggcaaacactggtcggaggcgtccccgcggcgcgcggcaggaagccaggccccaaccccctcccaaccgggcgccagccccgcctccgcccggttcaaacagcgaccgggtcgcgcgcgcgcacgcagcggccacaccctcgggcgccagcggctcgggcaggaagtggcgcaagcgcccgggccccagaacgcacgcgcgattagcgccattgagtcccagcgcgcacgcgcaattagcgccaattcccagcgcgcacgcagttagcgcccaaaggaccagcgcgcacgcgcatggcgccccagcccccaccgggcctgacgggggctacgccgcgcccaccgtgcgatccccattggcaagagcccggctcagacaaagaccccgccggttgcccccgccccgagagcggcacccccggagcgcgcccgcccgagcgcggcctcgcgcctgcgaactggcgtggggtgtcccccatctccggaggcccaggggcttctcccgcgccccccacggcggtccggttccgccccatgcgccccccgctgcggcccagacggcggctctgcacgggcgaagggccgcggccgcatgccccggtcggctggccgggcttacctggcggcgggtgtggacgggcggcggatcggcaaaggcgaggctctgtgctcgcgggcggacgcggtctcggcggtggtggcgcgtcgcgccgctgggttttatagggcgccgccgcggccgctcgagccataaaaggcaactttcggaacggcgcacgctgattggccccgcgccgctcactcaccggcttcgccgcacagtgcagcatttttttaccccctctcccctccttttgcgaaaaaaaaaaagagcgagagcgagattgaggaagaggaggagggagagttttggcgttggccgccttggggtgctgggcccgggggctgggggcgcgcgccgtggcccccgcgccccacgctgggcagtgcccggttcggccccgcatggccaggcctgcccccggcctgcccgtctctcgggccccccacccaccgcgggacatcctaggtgtggacatctcttgggcactgagcgcccaggtggggtgggccagggtctgcacgggtgccagggccctgggttctgtacgctcctgcagaaggagctcttggagggcatggagtggccaggcagtcactcccccttgccgacttcagagcaactgccctgaaagcagggcctgaggacctctggctgtggggctcagctagctaaatgtgctgggtgggtcactagggagagacctgggcttgagaggtagagtgtggtgttgggggagtcaggtggcttgcggccattagagtcgcaggaccacactccccaggacagggcaggggccagcggtccagtggctggaggtggcccgtgatgaaggctacaaacctacccagccgcagccctgggaaggaagtgggctctacagggcagggcaccttttaccctggagctgcctgcttttgagggtaacagtcacgcccagccaagaccaggcctggggcgttagtgggtgacctaggcactgcggggcgggggggctgggtctacacagcctgggtctgggcccaccgtccgttgtatgtctgctatgcgcagccacagctgaactgccctcccagaccatctggaggccgctgggggactctggggaccaagactccatgtgccacagaggattgggggcggggcggtgctaggaactcaaagccagcctgggaagaccctgtccttgtcaccctttcttgccttgggtctgtccactgagtagcacacaagaccgggtgggcagggtccgttctgctccgggaatcacagactgtgtgtacccaggtggtgggcatgcagcgatcagtggcgtgggaccacagagggggcccgcggtacctaaaacagcttcacatggcttaaaataggggaccaatgtcttttccaatctaagtcccatttataataaagtccatgttccatttttaaaggacaatcctttcggtttaaaaccaggcacgattacccaaacaactcacaacggtaaagcactgtgaatcttctctgttctgcaatcccaacttggtttctgctcagaaaccctccctctttccaatcggtaattaaataacaaaaggaaaaaacttaagatgcttcaaccccgtttcgtgacactttgaaaaaagaatcacctcttgcaaacacccgctcccgacccccgccgctgaagcccggcgtccagaggcctaagcgcgggtgcccgcccccacccgggagcgcgggcctcgtggtcagcgcatccgcggggagaaacaaaggccgcggcacgggggctcaagggcactgcgccacaccgcacgcgcctacccccgcgcggccacgttaactggcggtcgccgcagcctcgggacagccggccgcgcgccgccaggctcgcggacgcgggaccacgcgccgccctccgggaggcccaagtctcgacccagccccgcgtggcgctgggggagggggcgcctccgccggaacgcgggtgggggaggggagggggaaatgcgctttgtctcgaaatggggcaaccgtcgccacagctccctaccccctcgagggcagagcagtccccccactaactaccgggctggccgcgcgccaggccagccgcgaggccaccgcccgaccctccactccttcccgcagctcccggcgcggggtccggcgagaaggggaggggaggggagcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcctcggcgagcgtttcggcgggcagcaggtcctcgtgagcgaggctgcggagcttcccctccccctctctcccgggaaccgatttggcggccgccattttcatggctcgccttcctctcagcgttttccttataactcttttattttcttagtgtgctttctctatcaagaagtagaagtggttaactattttttttttcttctcgggctgttttcatatcgtttcgaggtggatttggagtgttttgtgagcttggatctttagagtcctgcgcacctcattaaaggcgctcagccttcccctcgatgaaatggcgccattgcgttcggaagccacaccgaagagcggggagggggggtgctccgggtttgcgggcccggtttcagagaagatatcaccacccagggcgtcgggccgggttcaatgcgagccgtaggacaaagaaaccattttatgtttttcctgtcttttttttcctttgagtaacggttttatctgggtctgcagtcagtaaaacgacagatgaaccgcggcaaaataaacataaattggaagccatcggccacgaggggcagggacgaaggtggttttctgggcgggggagggatattcgcgtcagaatcctttactgttcttaaggattccgtttaagttgtagagctgactcattttaagtaatgttgttactgagaagtttaacccttacgggacagatccatggacctttatagatgattacgaggaaagtgaaataacgattttgtccttagttatacttcgattaaaacatggcttcagaggctccttcctgtaatgcgtatggattgatgtgcaaaactgttttgggcctgggccgctctgtatttgaactttgttacttttctcattttgtttgcaatcttggttgaacattacattgataagcataaggtctcaagcgaagggggtctacctggttatttttctttgaccctaagcacgtttataaaataacattgtttaaaatcgatagtggacatcgggtaagtttggataaattgtgaggtaagtaatgagtttttgctttttgttagtgatttgtaaaacttgttataaatgtacattatccgtaatttcagtttagagataacctatgtgctgacgacaattaagaataaaaactagctgaaaaaatgaaaataactatcgtgacaagtaaccatttcaaaagactgctttgtgtctcataggagctagtttgatcatttcagttaattttttctttaatttttacgagtcatgaaaactacaggaaaaaaaatctgaactgggttttaccactactttttaggagttgggagcatgcgaatggagggagagctccgtagaactgggatgagagcagcaattaatgctgcttgctaggaacaaaaaataattgattgaaaattacgtgtgactttttagtttgcattatgcgtttgtagcagttggtcctggatatcactttctctcgtttgaggttttttaacctagttaacttttaagacaggtttccttaacattcataagtgcccagaatacagctgtgtagtacagcatataaagatttcagctctgaggtttttcctattgacttggaaaattgttttgtgcctgtcgcttgccacatggccaatcaagtaagcttcgaattcgagctcgcccaactccgcccgttttatgactagaaccaatagtttttaatgccaaatgcactgaaatcccctaatttgcaaagccaaacgccccctatgtgagtaatacggggactttttacccaatttcccaagcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcagcccgggtctagaggatccgcctgagaaaggaagtgagctgtaaaggctgagctctctctctgacgtatgtagcctctggttagcttcgtcactcactgttcttgactcagcatggcaatctgatgaaatcccagctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctccagatccactagttctagagcaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaccagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgtagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaaggggggggattgggragacaatagcaggcatgctgggggggcggtgggggctatggcttctgaggcggaaagaaccagctggggctcgagatccactagttctagcctcgaggctagagcggcctgctctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagggggtacctaggccgggcaacaattggcggccggccgcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccctaggccgggcaacaattggcggccggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactc

SEQ ID NO: 5 - DNA, 12704 bp, Artificial Sequence
  Artificial Sequence containing human UCOE elements and vector sequence
acgttgtaaaacgacggccagtgaattgtaatacgactcactatagggcgaattgggtaccgggccccccctcgaggtcgagttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtcgactctagacccgggctgcagcgaggagctctgcgttctacggtggtcagaccgaagactgcgacggtaccgacgctggtcgcgcctcttatacccacgtagaacgcagctcagccaatagaatgcgtgccaatatggaatttccaggggaaaaccggggcggtgttacgttttggctgccctttcacttcccattgacgtgtattggctcgagaacggtactttcccattaatcagctatgggaaagtaccgtttaaaggtcacgttgcattagtttcaatagcccattgacgtcaatggtgggaaagtacatggcgttttaattaaattggctggaaaaacccaatgactcacccctattgaccttatgtacgtgccaataatgggaaaaacccattgactcaccccctattgaccttttgtactgggcaaaacccaatggaaagtccctattgactcagtgtacttggctccaatgggactttcctgttgattcacccctattgaccttatgtactgggcaaaacccattggaaagtccctaatgactcagtatacgtgccagtaatgggaaaaacccattggcttacctcccattgaccttatgtactgggcaaaacccattggaaagtccctattgactcaatgtacttggctccaatgggactttcctgttgactcaccccctattgaccttatgtactgggcaaaacccaatggaaagtccctattgagtcagtgtacttggctccaatgggtttttcccattgactcatcccctattgaccttatgtactgggcaaaacccaatggaaagtccctattgacgcagtgtacttggctccaatgggactttcctgttgattcaccccctattgaccttatgtactgggcagaatacaatggaaagtccctattgactcacccacattgaccttatatgcttgccaacaatggaaaaacccattggaaagtccctattgagtcagtgtacttggcagcaatgggtttttcccattggctcacctcccattgacccaatgtacttgggcaaaacccagtggaaagtcccatttgactcagtgtgcttgccagtaatgggaaaaacccattggcttacctcccattgacccaatgtacttgggtaaaggccattgagtcaccacccctatgctgggaaatggtgaacgccccctatgtggaaagtccctatgggccgccattagagtgcatgaccgtgctgattcatatgccatatgagtgtattagggggctttccgcttgggaaattgggtaaaaagtccccgtattactcacatagggggcgtttggctttgcaaattaggggatttcagtgcatttggcattaaaaactattggttctagtcataaaacgggcggagttgggcgagctcgaattcaaacgactcgacggtatcaaggtggcgaccggaatggtgagctgcgagaatagccgggcgcgctgtgagccgaagtcgcccccgccctggccacttccggcgcgccgagtccttaggccgccagggggcgccggcgcgcgcccagattggggacaaaggaagccgggccggccgcgttattaccataaaaggcaaacactggtcggaggcgtccccgcggcgcgcggcaggaagccaggccccaaccccctcccaaccgggcgccagccccgcctccgcccggttcaaacagcgaccgggtcgcgcgcgcgcacgcagcggccacaccctcgggcgccagcggctcgggcaggaagtggcgcaagcgcccgggccccagaacgcacgcgcgattagcgccattgagtcccagcgcgcacgcgcaattagcgccaattcccagcgcgcacgcagttagcgcccaaaggaccagcgcgcacgcgcatggcgccccagcccccaccgggcctgacgggggctacgccgcgcccaccgtgcgatccccattggcaagagcccggctcagacaaagaccccgccggttgcccccgccccgagagcggcacccccggagcgcgcccgcccgagcgcggcctcgcgcctgcgaactggcgtggggtgtcccccatctccggaggcccaggggcttctcccgcgccccccacggcggtccggttccgccccatgcgccccccgctgcggcccagacggcggctctgcacgggcgaagggccgcggccgcatgccccggtcggctggccgggcttacctggcggcgggtgtggacgggcggcggatcggcaaaggcgaggctctgtgctcgcgggcggacgcggtctcggcggtggtggcgcgtcgcgccgctgggttttatagggcgccgccgcggccgctcgagccataaaaggcaactttcggaacggcgcacgctgattggccccgcgccgctcactcaccggcttcgccgcacagtgcagcatttttttaccccctctcccctccttttgcgaaaaaaaaaaagagcgagagcgagattgaggaagaggaggagggagagttttggcgttggccgccttggggtgctgggcccgggggctgggggcgcgcgccgtggcccccgcgccccacgctgggcagtgcccggttcggccccgcatggccaggcctgcccccggcctgcccgtctctcgggccccccacccaccgcgggacatcctaggtgtggacatctcttgggcactgagcgcccaggtggggtgggccagggtctgcacgggtgccagggccctgggttctgtacgctcctgcagaaggagctcttggagggcatggagtggccaggcagtcactcccccttgccgacttcagagcaactgccctgaaagcagggcctgaggacctctggctgtggggctcagctagctaaatgtgctgggtgggtcactagggagagacctgggcttgagaggtagagtgtggtgttgggggagtcaggtggcttgcggccattagagtcgcaggaccacactccccaggacagggcaggggccagcggtccagtggctggaggtggcccgtgatgaaggctacaaacctacccagccgcagccctgggaaggaagtgggctctacagggcagggcaccttttaccctggagctgcctgcttttgagggtaacagtcacgcccagccaagaccaggcctggggcgttagtgggtgacctaggcactgcggggcgggggggctgggtctacacagcctgggtctgggcccaccgtccgttgtatgtctgctatgcgcagccacagctgaactgccctcccagaccatctggaggccgctgggggactctggggaccaagactccatgtgccacagaggattgggggcggggcggtgctaggaactcaaagccagcctgggaagaccctgtccttgtcaccctttcttgccttgggtctgtccactgagtagcacacaagaccgggtgggcagggtccgttctgctccgggaatcacagactgtgtgtacccaggtggtgggcatgcagcgatcagtggcgtgggaccacagagggggcccgcggtacctaaaacagcttcacatggcttaaaataggggaccaatgtcttttccaatctaagtcccatttataataaagtccatgttccatttttaaaggacaatcctttcggtttaaaaccaggcacgattacccaaacaactcacaacggtaaagcactgtgaatcttctctgttctgcaatcccaacttggtttctgctcagaaaccctccctctttccaatcggtaattaaataacaaaaggaaaaaacttaagatgcttcaaccccgtttcgtgacactttgaaaaaagaatcacctcttgcaaacacccgctcccgacccccgccgctgaagcccggcgtccagaggcctaagcgcgggtgcccgcccccacccgggagcgcgggcctcgtggtcagcgcatccgcggggagaaacaaaggccgcggcacgggggctcaagggcactgcgccacaccgcacgcgcctacccccgcgcggccacgttaactggcggtcgccgcagcctcgggacagccggccgcgcgccgccaggctcgcggacgcgggaccacgcgccgccctccgggaggcccaagtctcgacccagccccgcgtggcgctgggggagggggcgcctccgccggaacgcgggtgggggaggggagggggaaatgcgctttgtctcgaaatggggcaaccgtcgccacagctccctaccccctcgagggcagagcagtccccccactaactaccgggctggccgcgcgccaggccagccgcgaggccaccgcccgaccctccactccttcccgcagctcccggcgcggggtccggcgagaaggggaggggaggggagcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcctcggcgagcgtttcggcgggcagcaggtcctcgtgagcgaggctgcggagcttcccctccccctctctcccgggaaccgatttggcggccgccattttcatggctcgccttcctctcagcgttttccttataactcttttattttcttagtgtgctttctctatcaagaagtagaagtggttaactattttttttttcttctcgggctgttttcatatcgtttcgaggtggatttggagtgttttgtgagcttggatctttagagtcctgcgcacctcattaaaggcgctcagccttcccctcgatgaaatggcgccattgcgttcggaagccacaccgaagagcggggagggggggtgctccgggtttgcgggcccggtttcagagaagatatcaccacccagggcgtcgggccgggttcaatgcgagccgtaggacaaagaaaccattttatgtttttcctgtcttttttttcctttgagtaacggttttatctgggtctgcagtcagtaaaacgacagatgaaccgcggcaaaataaacataaattggaagccatcggccacgaggggcagggacgaaggtggttttctgggcgggggagggatattcgcgtcagaatcctttactgttcttaaggattccgtttaagttgtagagctgactcattttaagtaatgttgttactgagaagtttaacccttacgggacagatccatggacctttatagatgattacgaggaaagtgaaataacgattttgtccttagttatacttcgattaaaacatggcttcagaggctccttcctgtaatgcgtatggattgatgtgcaaaactgttttgggcctgggccgctctgtatttgaactttgttacttttctcattttgtttgcaatcttggttgaacattacattgataagcataaggtctcaagcgaagggggtctacctggttatttttctttgaccctaagcacgtttataaaataacattgtttaaaatcgatagtggacatcgggtaagtttggataaattgtgaggtaagtaatgagtttttgctttttgttagtgatttgtaaaacttgttataaatgtacattatccgtaatttcagtttagagataacctatgtgctgacgacaattaagaataaaaactagctgaaaaaatgaaaataactatcgtgacaagtaaccatttcaaaagactgctttgtgtctcataggagctagtttgatcatttcagttaattttttctttaatttttacgagtcatgaaaactacaggaaaaaaaatctgaactgggttttaccactactttttaggagttgggagcatgcgaatggagggagagctccgtagaactgggatgagagcagcaattaatgctgcttgctaggaacaaaaaataattgattgaaaattacgtgtgactttttagtttgcattatgcgtttgtagcagttggtcctggatatcactttctctcgtttgaggttttttaacctagttaacttttaagacaggtttccttaacattcataagtgcccagaatacagctgtgtagtacagcatataaagatttcagctctgaggtttttcctattgacttggaaaattgttttgtgcctgtcgcttgccacatggccaatcaagtaagcttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcggatccgcctgagaaaggaagtgagctgtaaaggctgagctctctctctgacgtatgtagcctctggttagcttcgtcactcactgttcttgactcagcatggcaatctgatgaaatcccagctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctccagatccactagtaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcstggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgagactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagggggtacctaggccgggcaacaattggcggccggccgcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccctaggccgggcaacaattggcggccggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactc

SEQ ID NO: 6 - DNA, 11273 bp, Artificial Sequence
  Artificial Sequence containing human UCOE elements and vector sequence
acgttgtaaaacgacggccagtgaattgtaatacgactcactatagggcgaattgggtaccgggccccccctcgaggtcgagttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtcgacggtatcaaggtggcgaccggaatggtgagctgcgagaatagccgggcgcgctgtgagccgaagtcgcccccgccctggccacttccggcgcgccgagtccttaggccgccagggggcgccggcgcgcgcccagattggggacaaaggaagccgggccggccgcgttattaccataaaaggcaaacactggtcggaggcgtccccgcggcgcgcggcaggaagccaggccccaaccccctcccaaccgggcgccagccccgcctccgcccggttcaaacagcgaccgggtcgcgcgcgcgcacgcagcggccacaccctcgggcgccagcggctcgggcaggaagtggcgcaagcgcccgggccccagaacgcacgcgcgattagcgccattgagtcccagcgcgcacgcgcaattagcgccaattcccagcgcgcacgcagttagcgcccaaaggaccagcgcgcacgcgcatggcgccccagcccccaccgggcctgacgggggctacgccgcgcccaccgtgcgatccccattggcaagagcccggctcagacaaagaccccgccggttgcccccgccccgagagcggcacccccggagcgcgcccgcccgagcgcggcctcgcgcctgcgaactggcgtggggtgtcccccatctccggaggcccaggggcttctcccgcgccccccacggcggtccggttccgccccatgcgccccccgctgcggcccagacggcggctctgcacgggcgaagggccgcggccgcatgccccggtcggctggccgggcttacctggcggcgggtgtggacgggcggcggatcggcaaaggcgaggctctgtgctcgcgggcggacgcggtctcggcggtggtggcgcgtcgcgccgctgggttttatagggcgccgccgcggccgctcgagccataaaaggcaactttcggaacggcgcacgctgattggccccgcgccgctcactcaccggcttcgccgcacagtgcagcatttttttaccccctctcccctccttttgcgaaaaaaaaaaagagcgagagcgagattgaggaagaggaggagggagagttttggcgttggccgccttggggtgctgggcccgggggctgggggcgcgcgccgtggcccccgcgccccacgctgggcagtgcccggttcggccccgcatggccaggcctgcccccggcctgcccgtctctcgggccccccacccaccgcgggacatcctaggtgtggacatctcttgggcactgagcgcccaggtggggtgggccagggtctgcacgggtgccagggccctgggttctgtacgctcctgcagaaggagctcttggagggcatggagtggccaggcagtcactcccccttgccgacttcagagcaactgccctgaaagcagggcctgaggacctctggctgtggggctcagctagctaaatgtgctgggtgggtcactagggagagacctgggcttgagaggtagagtgtggtgttgggggagtcaggtggcttgcggccattagagtcgcaggaccacactccccaggacagggcaggggccagcggtccagtggctggaggtggcccgtgatgaaggctacaaacctacccagccgcagccctgggaaggaagtgggctctacagggcagggcaccttttaccctggagctgcctgcttttgagggtaacagtcacgcccagccaagaccaggcctggggcgttagtgggtgacctaggcactgcggggcgggggggctgggtctacacagcctgggtctgggcccaccgtccgttgtatgtctgctatgcgcagccacagctgaactgccctcccagaccatctggaggccgctgggggactctggggaccaagactccatgtgccacagaggattgggggcggggcggtgctaggaactcaaagccagcctgggaagaccctgtccttgtcaccctttcttgccttgggtctgtccactgagtagcacacaagaccgggtgggcagggtccgttctgctccgggaatcacagactgtgtgtacccaggtggtgggcatgcagcgatcagtggcgtgggaccacagagggggcccgcggtacctaaaacagcttcacatggcttaaaataggggaccaatgtcttttccaatctaagtcccatttataataaagtccatgttccatttttaaaggacaatcctttcggtttaaaaccaggcacgattacccaaacaactcacaacggtaaagcactgtgaatcttctctgttctgcaatcccaacttggtttctgctcagaaaccctccctctttccaatcggtaattaaataacaaaaggaaaaaacttaagatgcttcaaccccgtttcgtgacactttgaaaaaagaatcacctcttgcaaacacccgctcccgacccccgccgctgaagcccggcgtccagaggcctaagcgcgggtgcccgcccccacccgggagcgcgggcctcgtggtcagcgcatccgcggggagaaacaaaggccgcggcacgggggctcaagggcactgcgccacaccgcacgcgcctacccccgcgcggccacgttaactggcggtcgccgcagcctcgggacagccggccgcgcgccgccaggctcgcggacgcgggaccacgcgccgccctccgggaggcccaagtctcgacccagccccgcgtggcgctgggggagggggcgcctccgccggaacgcgggtgggggaggggagggggaaatgcgctttgtctcgaaatggggcaaccgtcgccacagctccctaccccctcgagggcagagcagtccccccactaactaccgggctggccgcgcgccaggccagccgcgaggccaccgcccgaccctccactccttcccgcagctcccggcgcggggtccggcgagaaggggaggggaggggagcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcctcggcgagcgtttcggcgggcagcaggtcctcgtgagcgaggctgcggagcttcccctccccctctctcccgggaaccgatttggcggccgccattttcatggctcgccttcctctcagcgttttccttataactcttttattttcttagtgtgctttctctatcaagaagtagaagtggttaactattttttttttcttctcgggctgttttcatatcgtttcgaggtggatttggagtgttttgtgagcttggatctttagagtcctgcgcacctcattaaaggcgctcagccttcccctcgatgaaatggcgccattgcgttcggaagccacaccgaagagcggggagggggggtgctccgggtttgcgggcccggtttcagagaagatatcaccacccagggcgtcgggccgggttcaatgcgagccgtaggacaaagaaaccattttatgtttttcctgtcttttttttcctttgagtaacggttttatctgggtctgcagtcagtaaaacgacagatgaaccgcggcaaaataaacataaattggaagccatcggccacgaggggcagggacgaaggtggttttctgggcgggggagggatattcgcgtcagaatcctttactgttcttaaggattccgtttaagttgtagagctgactcattttaagtaatgttgttactgagaagtttaacccttacgggacagatccatggacctttatagatgattacgaggaaagtgaaataacgattttgtccttagttatacttcgattaaaacatggcttcagaggctccttcctgtaatgcgtatggattgatgtgcaaaactgttttgggcctgggccgctctgtatttgaactttgttacttttctcattttgtttgcaatcttggttgaacattacattgataagcataaggtctcaagcgaagggggtctacctggttatttttctttgaccctaagcacgtttataaaataacattgtttaaaatcgatagtggacatcgggtaagtttggataaattgtgaggtaagtaatgagtttttgctttttgttagtgatttgtaaaacttgttataaatgtacattatccgtaatttcagtttagagataacctatgtgctgacgacaattaagaataaaaactagctgaaaaaatgaaaataactatcgtgacaagtaaccatttcaaaagactgctttgtgtctcataggagctagtttgatcatttcagttaattttttctttaatttttacgagtcatgaaaactacaggaaaaaaaatctgaactgggttttaccactactttttaggagttgggagcatgcgaatggagggagagctccgtagaactgggatgagagcagcaattaatgctgcttgctaggaacaaaaaataattgattgaaaattacgtgtgactttttagtttgcattatgcgtttgtagcagttggtcctggatatcactttctctcgtttgaggttttttaacctagttaacttttaagacaggtttccttaacattcataagtgcccagaatacagctgtgtagtacagcatataaagatttcagctctgaggtttttcctattgacttggaaaattgttttgtgcctgtcgcttgccacatggccaatcaagtaagcttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcggatccgcctgagaaaggaagtgagctgtaaaggctgagctctctctctgacgtatgtagcctctggttagcttcgtcactcactgttcttgactcagcatggcaatctgatgaaatcccagctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctccagatccactagtaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcstggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgagactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagggggtacctaggccgggcaacaattggcggccggccgcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccctaggccgggcaacaattggcggccggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactc

SEQ ID NO: 7 - DNA, 12591 bp, Artificial Sequence
  Artificial Sequence containing human UCOE elements and vector sequence
acgttgtaaaacgacggccagtgaattgtaatacgactcactatagggcgaattgggtaccgggccccccctcgaggtcgagttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtcgactctagacccgggctgcagcgaggagctctgcgttctacggtggtcagaccgaagactgcgacggtaccgacgctggtcgcgcctcttatacccacgtagaacgcagctcagccaatagaatgcgtgccaatatggaatttccaggggaaaaccggggcggtgttacgttttggctgccctttcacttcccattgacgtgtattggctcgagaacggtactttcccattaatcagctatgggaaagtaccgtttaaaggtcacgttgcattagtttcaatagcccattgacgtcaatggtgggaaagtacatggcgttttaattaaattggctggaaaaacccaatgactcacccctattgaccttatgtacgtgccaataatgggaaaaacccattgactcaccccctattgaccttttgtactgggcaaaacccaatggaaagtccctattgactcagtgtacttggctccaatgggactttcctgttgattcacccctattgaccttatgtactgggcaaaacccattggaaagtccctaatgactcagtatacgtgccagtaatgggaaaaacccattggcttacctcccattgaccttatgtactgggcaaaacccattggaaagtccctattgactcaatgtacttggctccaatgggactttcctgttgactcaccccctattgaccttatgtactgggcaaaacccaatggaaagtccctattgagtcagtgtacttggctccaatgggtttttcccattgactcatcccctattgaccttatgtactgggcaaaacccaatggaaagtccctattgacgcagtgtacttggctccaatgggactttcctgttgattcaccccctattgaccttatgtactgggcagaatacaatggaaagtccctattgactcacccacattgaccttatatgcttgccaacaatggaaaaacccattggaaagtccctattgagtcagtgtacttggcagcaatgggtttttcccattggctcacctcccattgacccaatgtacttgggcaaaacccagtggaaagtcccatttgactcagtgtgcttgccagtaatgggaaaaacccattggcttacctcccattgacccaatgtacttgggtaaaggccattgagtcaccacccctatgctgggaaatggtgaacgccccctatgtggaaagtccctatgggccgccattagagtgcatgaccgtgctgattcatatgccatatgagtgtattagggggctttccgcttgggaaattgggtaaaaagtccccgtattactcacatagggggcgtttggctttgcaaattaggggatttcagtgcatttggcattaaaaactattggttctagtcataaaacgggcggagttgggcgagctcgaattcaaacgactcgacggtatcaaggtggcgaccggaatggtgagctgcgagaatagccgggcgcgctgtgagccgaagtcgcccccgccctggccacttccggcgcgccgagtccttaggccgccagggggcgccggcgcgcgcccagattggggacaaaggaagccgggccggccgcgttattaccataaaaggcaaacactggtcggaggcgtccccgcggcgcgcggcaggaagccaggccccaaccccctcccaaccgggcgccagccccgcctccgcccggttcaaacagcgaccgggtcgcgcgcgcgcacgcagcggccacaccctcgggcgccagcggctcgggcaggaagtggcgcaagcgcccgggccccagaacgcacgcgcgattagcgccattgagtcccagcgcgcacgcgcaattagcgccaattcccagcgcgcacgcagttagcgcccaaaggaccagcgcgcacgcgcatggcgccccagcccccaccgggcctgacgggggctacgccgcgcccaccgtgcgatccccattggcaagagcccggctcagacaaagaccccgccggttgcccccgccccgagagcggcacccccggagcgcgcccgcccgagcgcggcctcgcgcctgcgaactggcgtggggtgtcccccatctccggaggcccaggggcttctcccgcgccccccacggcggtccggttccgccccatgcgccccccgctgcggcccagacggcggctctgcacgggcgaagggccgcggccgcatgccccggtcggctggccgggcttacctggcggcgggtgtggacgggcggcggatcggcaaaggcgaggctctgtgctcgcgggcggacgcggtctcggcggtggtggcgcgtcgcgccgctgggttttatagggcgccgccgcggccgctcgagccataaaaggcaactttcggaacggcgcacgctgattggccccgcgccgctcactcaccggcttcgccgcacagtgcagcatttttttaccccctctcccctccttttgcgaaaaaaaaaaagagcgagagcgagattgaggaagaggaggagggagagttttggcgttggccgccttggggtgctgggcccgggggctgggggcgcgcgccgtggcccccgcgccccacgctgggcagtgcccggttcggccccgcatggccaggcctgcccccggcctgcccgtctctcgggccccccacccaccgcgggacatcctaggtgtggacatctcttgggcactgagcgcccaggtggggtgggccagggtctgcacgggtgccagggccctgggttctgtacgctcctgcagaaggagctcttggagggcatggagtggccaggcagtcactcccccttgccgacttcagagcaactgccctgaaagcagggcctgaggacctctggctgtggggctcagctagctaaatgtgctgggtgggtcactagggagagacctgggcttgagaggtagagtgtggtgttgggggagtcaggtggcttgcggccattagagtcgcaggaccacactccccaggacagggcaggggccagcggtccagtggctggaggtggcccgtgatgaaggctacaaacctacccagccgcagccctgggaaggaagtgggctctacagggcagggcaccttttaccctggagctgcctgcttttgagggtaacagtcacgcccagccaagaccaggcctggggcgttagtgggtgacctaggcactgcggggcgggggggctgggtctacacagcctgggtctgggcccaccgtccgttgtatgtctgctatgcgcagccacagctgaactgccctcccagaccatctggaggccgctgggggactctggggaccaagactccatgtgccacagaggattgggggcggggcggtgctaggaactcaaagccagcctgggaagaccctgtccttgtcaccctttcttgccttgggtctgtccactgagtagcacacaagaccgggtgggcagggtccgttctgctccgggaatcacagactgtgtgtacccaggtggtgggcatgcagcgatcagtggcgtgggaccacagagggggcccgcggtacctaaaacagcttcacatggcttaaaataggggaccaatgtcttttccaatctaagtcccatttataataaagtccatgttccatttttaaaggacaatcctttcggtttaaaaccaggcacgattacccaaacaactcacaacggtaaagcactgtgaatcttctctgttctgcaatcccaacttggtttctgctcagaaaccctccctctttccaatcggtaattaaataacaaaaggaaaaaacttaagatgcttcaaccccgtttcgtgacactttgaaaaaagaatcacctcttgcaaacacccgctcccgacccccgccgctgaagcccggcgtccagaggcctaagcgcgggtgcccgcccccacccgggagcgcgggcctcgtggtcagcgcatccgcggggagaaacaaaggccgcggcacgggggctcaagggcactgcgccacaccgcacgcgcctacccccgcgcggccacgttaactggcggtcgccgcagcctcgggacagccggccgcgcgccgccaggctcgcggacgcgggaccacgcgccgccctccgggaggcccaagtctcgacccagccccgcgtggcgctgggggagggggcgcctccgccggaacgcgggtgggggaggggagggggaaatgcgctttgtctcgaaatggggcaaccgtcgccacagctccctaccccctcgagggcagagcagtccccccactaactaccgggctggccgcgcgccaggccagccgcgaggccaccgcccgaccctccactccttcccgcagctcccggcgcggggtccggcgagaaggggaggggaggggagcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcctcggcgagcgtttcggcgggcagcaggtcctcgtgagcgaggctgcggagcttcccctccccctctctcccgggaaccgatttggcggccgccattttcatggctcgccttcctctcagcgttttccttataactcttttattttcttagtgtgctttctctatcaagaagtagaagtggttaactattttttttttcttctcgggctgttttcatatcgtttcgaggtggatttggagtgttttgtgagcttggatctttagagtcctgcgcacctcattaaaggcgctcagccttcccctcgatgaaatggcgccattgcgttcggaagccacaccgaagagcggggagggggggtgctccgggtttgcgggcccggtttcagagaagatatcaccacccagggcgtcgggccgggttcaatgcgagccgtaggacaaagaaaccattttatgtttttcctgtcttttttttcctttgagtaacggttttatctgggtctgcagtcagtaaaacgacagatgaaccgcggcaaaataaacataaattggaagccatcggccacgaggggcagggacgaaggtggttttctgggcgggggagggatattcgcgtcagaatcctttactgttcttaaggattccgtttaagttgtagagctgactcattttaagtaatgttgttactgagaagtttaacccttacgggacagatccatggacctttatagatgattacgaggaaagtgaaataacgattttgtccttagttatacttcgattaaaacatggcttcagaggctccttcctgtaatgcgtatggattgatgtgcaaaactgttttgggcctgggccgctctgtatttgaactttgttacttttctcattttgtttgcaatcttggttgaacattacattgataagcataaggtctcaagcgaagggggtctacctggttatttttctttgaccctaagcacgtttataaaataacattgtttaaaatcgatagtggacatcgggtaagtttggataaattgtgaggtaagtaatgagtttttgctttttgttagtgatttgtaaaacttgttataaatgtacattatccgtaatttcagtttagagataacctatgtgctgacgacaattaagaataaaaactagctgaaaaaatgaaaataactatcgtgacaagtaaccatttcaaaagactgctttgtgtctcataggagctagtttgatcatttcagttaattttttctttaatttttacgagtcatgaaaactacaggaaaaaaaatctgaactgggttttaccactactttttaggagttgggagcatgcgaatggagggagagctccgtagaactgggatgagagcagcaattaatgctgcttgctaggaacaaaaaataattgattgaaaattacgtgtgactttttagtttgcattatgcgtttgtagcagttggtcctggatatcactttctctcgtttgaggttttttaacctagttaacttttaagacaggtttccttaacattcataagtgcccagaatacagctgtgtagtacagcatataaagatttcagctctgaggtttttcctattgacttggaaaattgttttgtgcctgtcgcttgccacatggccaatcaagtaagcttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcggatccgcctgagaaaggaagtgagctgtaaaggctgagctctctctctgacgtatgtagcctctggttagcttcgtcactcactgttcttgactcagcatggcaatctgatgaaatcccagctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctccagatccactagttctagagcaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaccagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagaacagatggaaggcctcctggcgccgcaccggcccaaggagcccgcgtggttcctggccaccgtcgcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcataggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgagatctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagggggtacctaggccgggcaacaattggcggccggccgcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccctaggccgggcaacaattggcggccggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactc

SEQ ID NO: 8 - DNA, 11160 bp, Artificial Sequence
  Artificial Sequence containing human UCOE elements and vector sequence
acgttgtaaaacgacggccagtgaattgtaatacgactcactatagggcgaattgggtaccgggccccccctcgaggtcgagttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtcgacggtatcaaggtggcgaccggaatggtgagctgcgagaatagccgggcgcgctgtgagccgaagtcgcccccgccctggccacttccggcgcgccgagtccttaggccgccagggggcgccggcgcgcgcccagattggggacaaaggaagccgggccggccgcgttattaccataaaaggcaaacactggtcggaggcgtccccgcggcgcgcggcaggaagccaggccccaaccccctcccaaccgggcgccagccccgcctccgcccggttcaaacagcgaccgggtcgcgcgcgcgcacgcagcggccacaccctcgggcgccagcggctcgggcaggaagtggcgcaagcgcccgggccccagaacgcacgcgcgattagcgccattgagtcccagcgcgcacgcgcaattagcgccaattcccagcgcgcacgcagttagcgcccaaaggaccagcgcgcacgcgcatggcgccccagcccccaccgggcctgacgggggctacgccgcgcccaccgtgcgatccccattggcaagagcccggctcagacaaagaccccgccggttgcccccgccccgagagcggcacccccggagcgcgcccgcccgagcgcggcctcgcgcctgcgaactggcgtggggtgtcccccatctccggaggcccaggggcttctcccgcgccccccacggcggtccggttccgccccatgcgccccccgctgcggcccagacggcggctctgcacgggcgaagggccgcggccgcatgccccggtcggctggccgggcttacctggcggcgggtgtggacgggcggcggatcggcaaaggcgaggctctgtgctcgcgggcggacgcggtctcggcggtggtggcgcgtcgcgccgctgggttttatagggcgccgccgcggccgctcgagccataaaaggcaactttcggaacggcgcacgctgattggccccgcgccgctcactcaccggcttcgccgcacagtgcagcatttttttaccccctctcccctccttttgcgaaaaaaaaaaagagcgagagcgagattgaggaagaggaggagggagagttttggcgttggccgccttggggtgctgggcccgggggctgggggcgcgcgccgtggcccccgcgccccacgctgggcagtgcccggttcggccccgcatggccaggcctgcccccggcctgcccgtctctcgggccccccacccaccgcgggacatcctaggtgtggacatctcttgggcactgagcgcccaggtggggtgggccagggtctgcacgggtgccagggccctgggttctgtacgctcctgcagaaggagctcttggagggcatggagtggccaggcagtcactcccccttgccgacttcagagcaactgccctgaaagcagggcctgaggacctctggctgtggggctcagctagctaaatgtgctgggtgggtcactagggagagacctgggcttgagaggtagagtgtggtgttgggggagtcaggtggcttgcggccattagagtcgcaggaccacactccccaggacagggcaggggccagcggtccagtggctggaggtggcccgtgatgaaggctacaaacctacccagccgcagccctgggaaggaagtgggctctacagggcagggcaccttttaccctggagctgcctgcttttgagggtaacagtcacgcccagccaagaccaggcctggggcgttagtgggtgacctaggcactgcggggcgggggggctgggtctacacagcctgggtctgggcccaccgtccgttgtatgtctgctatgcgcagccacagctgaactgccctcccagaccatctggaggccgctgggggactctggggaccaagactccatgtgccacagaggattgggggcggggcggtgctaggaactcaaagccagcctgggaagaccctgtccttgtcaccctttcttgccttgggtctgtccactgagtagcacacaagaccgggtgggcagggtccgttctgctccgggaatcacagactgtgtgtacccaggtggtgggcatgcagcgatcagtggcgtgggaccacagagggggcccgcggtacctaaaacagcttcacatggcttaaaataggggaccaatgtcttttccaatctaagtcccatttataataaagtccatgttccatttttaaaggacaatcctttcggtttaaaaccaggcacgattacccaaacaactcacaacggtaaagcactgtgaatcttctctgttctgcaatcccaacttggtttctgctcagaaaccctccctctttccaatcggtaattaaataacaaaaggaaaaaacttaagatgcttcaaccccgtttcgtgacactttgaaaaaagaatcacctcttgcaaacacccgctcccgacccccgccgctgaagcccggcgtccagaggcctaagcgcgggtgcccgcccccacccgggagcgcgggcctcgtggtcagcgcatccgcggggagaaacaaaggccgcggcacgggggctcaagggcactgcgccacaccgcacgcgcctacccccgcgcggccacgttaactggcggtcgccgcagcctcgggacagccggccgcgcgccgccaggctcgcggacgcgggaccacgcgccgccctccgggaggcccaagtctcgacccagccccgcgtggcgctgggggagggggcgcctccgccggaacgcgggtgggggaggggagggggaaatgcgctttgtctcgaaatggggcaaccgtcgccacagctccctaccccctcgagggcagagcagtccccccactaactaccgggctggccgcgcgccaggccagccgcgaggccaccgcccgaccctccactccttcccgcagctcccggcgcggggtccggcgagaaggggaggggaggggagcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcctcggcgagcgtttcggcgggcagcaggtcctcgtgagcgaggctgcggagcttcccctccccctctctcccgggaaccgatttggcggccgccattttcatggctcgccttcctctcagcgttttccttataactcttttattttcttagtgtgctttctctatcaagaagtagaagtggttaactattttttttttcttctcgggctgttttcatatcgtttcgaggtggatttggagtgttttgtgagcttggatctttagagtcctgcgcacctcattaaaggcgctcagccttcccctcgatgaaatggcgccattgcgttcggaagccacaccgaagagcggggagggggggtgctccgggtttgcgggcccggtttcagagaagatatcaccacccagggcgtcgggccgggttcaatgcgagccgtaggacaaagaaaccattttatgtttttcctgtcttttttttcctttgagtaacggttttatctgggtctgcagtcagtaaaacgacagatgaaccgcggcaaaataaacataaattggaagccatcggccacgaggggcagggacgaaggtggttttctgggcgggggagggatattcgcgtcagaatcctttactgttcttaaggattccgtttaagttgtagagctgactcattttaagtaatgttgttactgagaagtttaacccttacgggacagatccatggacctttatagatgattacgaggaaagtgaaataacgattttgtccttagttatacttcgattaaaacatggcttcagaggctccttcctgtaatgcgtatggattgatgtgcaaaactgttttgggcctgggccgctctgtatttgaactttgttacttttctcattttgtttgcaatcttggttgaacattacattgataagcataaggtctcaagcgaagggggtctacctggttatttttctttgaccctaagcacgtttataaaataacattgtttaaaatcgatagtggacatcgggtaagtttggataaattgtgaggtaagtaatgagtttttgctttttgttagtgatttgtaaaacttgttataaatgtacattatccgtaatttcagtttagagataacctatgtgctgacgacaattaagaataaaaactagctgaaaaaatgaaaataactatcgtgacaagtaaccatttcaaaagactgctttgtgtctcataggagctagtttgatcatttcagttaattttttctttaatttttacgagtcatgaaaactacaggaaaaaaaatctgaactgggttttaccactactttttaggagttgggagcatgcgaatggagggagagctccgtagaactgggatgagagcagcaattaatgctgcttgctaggaacaaaaaataattgattgaaaattacgtgtgactttttagtttgcattatgcgtttgtagcagttggtcctggatatcactttctctcgtttgaggttttttaacctagttaacttttaagacaggtttccttaacattcataagtgcccagaatacagctgtgtagtacagcatataaagatttcagctctgaggtttttcctattgacttggaaaattgttttgtgcctgtcgcttgccacatggccaatcaagtaagcttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcggatccgcctgagaaaggaagtgagctgtaaaggctgagctctctctctgacgtatgtagcctctggttagcttcgtcactcactgttcttgactcagcatggcaatctgatgaaatcccagctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctccagatccactagttctagagcaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaccagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagaacagatggaaggcctcctggcgccgcaccggcccaaggagcccgcgtggttcctggccaccgtcgcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcataggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgagatctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagggggtacctaggccgggcaacaattggcggccggccgcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccctaggccgggcaacaattggcggccggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactc

SEQ ID NO: 9 - DNA, 14262 bp, Artificial Sequence
  Artificial Sequence containing human UCOE elements and vector sequence
ggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgaggtcgacggtatcgataagcttcaatgtttttagcaccctctgtgtggaggaaaataatgcagattattctaattagtgtaatatctaaccacattaaaatatattacatagtaaactacactccataattttataaatttgactccccagggtaataaactagtctctagtctgctcaccttcaactgtacaataaagtcttggttcttttgaaatagacctcaaatgagacacctaaaattcaaagtgtctttacatttaaagacacctacaggaaagcaggtaaaagagccaggttaaaaacaaattctaaaaccacttagctgcagttaaacatatagtaaagatgcactaaagtttcttactctgtaaatcccttccacttcaggaaatattccactttcccattcactacacgtcgatctagtactttttccacgacaaattcttcaggctctgcctcttcaacttttttactctttccattctgtttttttcccattttttgctaaaataaaacaaaagagaaattaagaaatattcctcttgaattttgagcacattttcaaggctcaattgcttatattattatcacattcgacataaatttttacttctatatcccagggcagacaccttctggaaagattaaaagtcaacagacaataaaataaaagaatgctttatcttgttcatttagttcaaacttacaacccaccaccaaaataatacaataaaaaaacactatctggaaacagttatttttttccagtctttttttttgagacagggtctcacactcttgtcgcccaggctggagtgcagtggcgtgatctcagctcactgcaacctccgcctccccaggttcaagcagttctcatgcctcagcctccagagtagctgggattataggcggatgccaccatgccgggctaattttttttgtgtttttattagaaacagggtttcaccatgttgaccaggctggtctcaaactcctgacctgaagtgattcaccagcctgggcctcccaaagtgctggcattacaggcgtgagccactgcgcccggccctgtagtcttaaaagaccaagtttactaattttcactcattttaacaacactgcaacaaacaactatgcaggaagtacctaaagggtgatccagagaagcaagtagtagtgacaggtcttaggtgaacctatgacagaccttgtatccacccccagatggtaaaagccccagcccccttctcaattcaaatattaatgtcaaaagcatcaatgatacagagaaaagataaatgcagaatgaaaacatggttcaaaatcctgataccaactgcagggtcaactatagagaccactaggaggttcaattaaaggacaagattatttttccataatctctgtagataatatttcctaccacttagaacaaaactataaagctatcacttcaagagaccaacattacaaatttattttaattccctaaggtgaaaaaaatccttccttcctggtttctcaagagaaagtctatactggtaaccaaattcactttaaacaggcattttctttggtatgacactatttaagagaagcaggaaaccaacgtgaaccagctctttccaatggctcaagatttcctatgagaggactaaaaatggggaaaatttttatgagaggattaaaaatgggggaaaaaaaaccctgaaatggttaatcagaagatcctatgggctgagaaggaatccatcttaacatttcatcttaaagcaaatgctattgccgggggcagtggctcatgcctgtaatcccagcactttgggaggccgaggtgggcagatcatctgaggtcaggagtttgagaccagcctgaccaacatggagaaaccccgtttctactaaaaatacaaaattagccaggcatagtggtgcatgcctgtaatcccagctacttgggaggctgaggcaggagaactgcttgaacccaggaggcttaagttgcggtgagccaagatcacgccattgcactctagcctggacaacaagagaaaaactctgtctcaaaaaaacacaaaaacaaaaaacccaaatactatttaaaaaagataaaccttaattgctcaatcattaaagccatcccacaagtaaagcagcaagcagaaaaaagttaagaacacctcaaggctacagaaggacatttcaagctatgcaggcatatgaagtgtgcagacagatatgtaagaaaggcctcaagactgcaaaagggcatttcaagctatgcaagcatataggtaacacatacacacacacaaaataaaatcccctgaaatacaaaaacatgcagcaaacacctgacgtttttggataccatttctaagtcaggtgttatgattctcattagtcaagatacttgagtactgggcccaaacagctttctgccactgtacagtacaagaaggtaggaataatggtgggaggagcaaagacaaactgtaatagacagaagtgtatcagatacctatactacatgaaaaacaaaacagctactgccacaaagggagaaggctaacaaaataaagtcaacaataaatacagaaaatgaaaaggatacacactaaggtttacaaaaaaaaaaaggcagacaaaatgccatacagtattcattcactactatggcattcataagctagtttcaaatgctcactattttcttttatagtatatatttgccttaacccagcacttttttccaaaagtggatgagtcaaaataaatttcccattatttaagtgaaattaacagcacacatatctcacaacactaatgaatttttaaaatggaaagttaagaacttttaaagtggccaacctgtgatccttcacaaaataaactaaatacaataacagaccccaaaggctatcaattgcgtgcaaaaacaacttctgttttccagggtaaacagaatctaatgcagaatctaatgcagggtaaacagacttaatgcagaatctaatgatggcacaaattaaaaatcactaacgtgccctttttagtgtgaaacccagagagagcacatacaagccaaaaacaaatgctttattttacctaggagacattaacattcacctttacgtgtttaagattaatgcaatgttaaatattgtgaaaactgtaactttgaatttcatgatttttatgtgaatattccagggtttaaaaaaacttgtaacatgacatggctgaataagataaaaaaaaaatctagccttttctcccttctggctcatatttgcgatttcgatcattttgtttaaaaaacaaaacactgcaatgaattaaacttaatattcttctatgttttagagtaagttaaaacaagataaagtgaccaaagtaatttgaaagattcaatgacttttgctccaacctaggtgcacaaggtaccttgttctttaaattgggctttaatgaaaatacttctccagaattctggggatttaagaaaaattatgccaaccaacaagggctttaccattttatgtaacatttttcaacgctgcaaaaatgtgtgtatttctatttgaagataaaaatcctcagcaaaatccacattgcactgtccttcaaagattagccttctttgaactagttaagacactattaagccaagccagtatctccctgtaatgaattcgtttttctcttaattttcccctgtaatttacactgggagagctgggaaatatgtggatgtaaatttctcagccacagagatgcaaagttatactgtggggaaaaaaaacttgagttaaatccttacatattttaggttttcattaacttaccaatgtagttttgttggaggccattttttttattgcagacttgaagagctattactagaaaaatgcatgacagttaaggtaagtttgcatgacacaaaaaaggtaactaaatacaaattctgtttggattccaacccccaagtagagagcgcacactttcaaacgtgaatacaaatccagagtagatctgcgctcctacctacattgcttatgatgtacttaagtacgtgtcctaaccatgtgagtctagaaagactttactggggatcctggtacctaaaacagcttcacatggcttaaaataggggaccaatgtcttttccaatctaagtcccatttataataaagtccatgttccatttttaaaggacaatcctttcggtttaaaaccaggcacgattacccaaacaactcacaacggtaaagcactgtgaatcttctctgttctgcaatcccaacttggtttctgctcagaaaccctccctctttccaatcggtaattaaataacaaaaggaaaaaacttaagatgcttcaaccccgtttcgtgacactttgaaaaaagaatcacctcttgcaaacacccgctcccgacccccgccgctgaagcccggcgtccagaggcctaagcgcgggtgcccgcccccacccgggagcgcgggcctcgtggtcagcgcatccgcggggagaaacaaaggccgcggcacgggggctcaagggcactgcgccacaccgcacgcgcctacccccgcgcggccacgttaactggcggtcgccgcagcctcgggacagccggccgcgcgccgccaggctcgcggacgcgggaccacgcgccgccctccgggaggcccaagtctcgacccagccccgcgtggcgctgggggagggggcgcctccgccggaacgcgggtgggggaggggagggggaaatgcgctttgtctcgaaatggggcaaccgtcgccacagctccctaccccctcgagggcagagcagtccccccactaactaccgggctggccgcgcgccaggccagccgcgaggccaccgcccgaccctccactccttcccgcagctcccggcgcggggtccggcgagaaggggaggggaggggagcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcctcggcgagcgtttcggcgggcagcaggtcctcgtgagcgaggctgcggagcttcccctccccctctctcccgggaaccgatttggcggccgccattttcatggctcgccttcctctcagcgttttccttataactcttttattttcttagtgtgctttctctatcaagaagtagaagtggttaactattttttttttcttctcgggctgttttcatatcgtttcgaggtggatttggagtgttttgtgagcttggatctttagagtcctgcgcacctcattaaaggcgctcagccttcccctcgatgaaatggcgccattgcgttcggaagccacaccgaagagcggggagggggggtgctccgggtttgcgggcccggtttcagagaagatatcaccacccagggcgtcgggccgggttcaatgcgagccgtaggacaaagaaaccattttatgtttttcctgtcttttttttcctttgagtaacggttttatctgggtctgcagtcagtaaaacgacagatgaaccgcggcaaaataaacataaattggaagccatcggccacgaggggcagggacgaaggtggttttctgggcgggggagggatattcgcgtcagaatcctttactgttcttaaggattccgtttaagttgtagagctgactcattttaagtaatgttgttactgagaagtttaacccttacgggacagatccatggacctttatagatgattacgaggaaagtgaaataacgattttgtccttagttatacttcgattaaaacatggcttcagaggctccttcctgtaatgcgtatggattgatgtgcaaaactgttttgggcctgggccgctctgtatttgaactttgttacttttctcattttgtttgcaatcttggttgaacattacattgataagcataaggtctcaagcgaagggggtctacctggttatttttctttgaccctaagcacgtttataaaataacattgtttaaaatcgatagtggacatcgggtaagtttggataaattgtgaggtaagtaatgagtttttgctttttgttagtgatttgtaaaacttgttataaatgtacattatccgtaatttcagtttagagataacctatgtgctgacgacaattaagaataaaaactagctgaaaaaatgaaaataactatcgtgacaagtaaccatttcaaaagactgctttgtgtctcataggagctagtttgatcatttcagttaattttttctttaatttttacgagtcatgaaaactacaggaaaaaaaatctgaactgggttttaccactactttttaggagttgggagcatgcgaatggagggagagctccgtagaactgggatgagagcagcaattaatgctgcttgctaggaacaaaaaataattgattgaaaattacgtgtgactttttagtttgcattatgcgtttgtagcagttggtcctggatatcactttctctcgtttgaggttttttaacctagttaacttttaagacaggtttccttaacattcataagtgcccagaatacagctgtgtagtacagcatataaagatttcagctctgaggtttttcctattgacttggaaaattgttttgtgcctgtcgcttgccacatggccaatcaagtaagcttgattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaaatcgaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaccagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagaacagatggaaggcctcctggcgccgcaccggcccaaggagcccgcgtggttcctggccaccgtcgcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcatcgtagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaaggggggggattgggragacaatagcaggcatgctgggggggcggtgggggctatggcttctgaggcggaaagaaccagctggggctcgagatccactagttctagcctcgaggctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcag

SEQ ID NO: 10 - DNA, 13 bp, Artificial Sequence
  PCR primer
aacaattggcggc

SEQ ID NO: 11 - DNA, 13 bp, Artificial Sequence
  PCR primer
gccaattgttgcc

SEQ ID NO: 12 - DNA, 31 bp, Artificial Sequence
  PCR primer
acgcgtcgacggaaggagacaataccggaag

SEQ ID NO: 13 - DNA, 28 bp, Artificial Sequence
  PCR primer
ccgctcgagttggggtggggaaaaggaa

SEQ ID NO: 14 - DNA, 30 bp, Artificial Sequence
  PCR primer
cgggatccgcctgagaaaggaagtgagctg

SEQ ID NO: 15 - DNA, 29 bp, Artificial Sequence
  PCR primer
gaagatctggaggaatgagctggccctta

SEQ ID NO: 16 - DNA, 8 bp, Artificial Sequence
  PCR primer
gactagtc

SEQ ID NO: 17 - DNA, 35 bp, Artificial Sequence
  PCR primer
ctcgagttattaatagtaatcaattacggggtcat

SEQ ID NO: 18 - DNA, 33 bp, Artificial Sequence
  PCR primer
gtcgacgatctgacggttcactaaaccagctct

SEQ ID NO: 19 - DNA, 30 bp, Artificial Sequence
  PCR primer
ccaatgcataggttgggcttcgggaatcgt

SEQ ID NO: 20 - DNA, 31 bp, Artificial Sequence
  PCR primer
gctctagatctcgacggtatacagacatgat

SEQ ID NO: 21 - DNA, 36 bp, Artificial Sequence
  PCR primer
cccaagcttattaatagtaatcaattacggggtcat

SEQ ID NO: 22 - DNA, 36 bp, Artificial Sequence
  PCR primer
caaggatccgatctgacggttcactaaaccagctct

SEQ ID NO: 23 - DNA, 20 bp, Artificial Sequence
  PCR primer
tcgagtcgtttaaactctag

SEQ ID NO: 24 - DNA, 20 bp, Artificial Sequence
  PCR primer
tcgactagagtttaaacgac

SEQ ID NO: 25 - DNA, 33 bp, Artificial Sequence
  PCR primer
gaattcgagctcgcccaactccgcccgttttat

SEQ ID NO: 26 - DNA, 39 bp, Artificial Sequence
  PCR primer
atttgtcgactctagacccgggctgcagcgaggagctct

SEQ ID NO: 27 - DNA, 12588 bp, Artificial Sequence
  Artificial Sequence containing human UCOE elements and vector sequence
acgttgtaaaacgacggccagtgaattgtaatacgactcactatagggcgaattgggtaccgggccccccctcgaggtcgagttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtcgacgatctgacggttcactaaaccagctctgcttatatagacctcccaccgtacacgcctaccgcccatttgcgtcaatggggcggagttgttacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataactcgacggtatcatggtggcgaccggcatggtgagctgcgagaatagccgggcgcgctgtgagccgaagtcgcccccgccctggccacttccggcgcgccgagtccttaggccgccagggggcgccggcgcgcgcccagattggggacaaaggaagccgggccggccgcgttattaccataaaaggcaaacactggtcggaggcgtccccgcggcgcgcggcaggaagccaggccccaaccccctcccaaccgggcgccagccccgcctccgcccggttcaaacagcgaccgggtcgcgcgcgcgcacgcagcggccacaccctcgggcgccagcggctcgggcaggaagtggcgcaagcgcccgggccccagaacgcacgcgcgattagcgccattgagtcccagcgcgcacgcgcaattagcgccaattcccagcgcgcacgcagttagcgcccaaaggaccagcgcgcacgcgcatggcgccccagcccccaccgggcctgacgggggctacgccgcgcccaccgtgcgatccccattggcaagagcccggctcagacaaagaccccgccggttgcccccgccccgagagcggcacccccggagcgcgcccgcccgagcgcggcctcgcgcctgcgaactggcgtggggtgtcccccatctccggaggcccaggggcttctcccgcgccccccacggcggtccggttccgccccatgcgccccccgctgcggcccagacggcggctctgcacgggcgaagggccgcggccgcatgccccggtcggctggccgggcttacctggcggcgggtgtggacgggcggcggatcggcaaaggcgaggctctgtgctcgcgggcggacgcggtctcggcggtggtggcgcgtcgcgccgctgggttttatagggcgccgccgcggccgctcgagccataaaaggcaactttcggaacggcgcacgctgattggccccgcgccgctcactcaccggcttcgccgcacagtgcagcatttttttaccccctctcccctccttttgcgaaaaaaaaaaagagcgagagcgagattgaggaagaggaggagggagagttttggcgttggccgccttggggtgctgggcccgggggctgggggcgcgcgccgtggcccccgcgccccacgctgggcagtgcccggttcggccccgcatggccaggcctgcccccggcctgcccgtctctcgggccccccacccaccgcgggacatcctaggtgtggacatctcttgggcactgagcgcccaggtggggtgggccagggtctgcacgggtgccagggccctgggttctgtacgctcctgcagaaggagctcttggagggcatggagtggccaggcagtcactcccccttgccgacttcagagcaactgccctgaaagcagggcctgaggacctctggctgtggggctcagctagctaaatgtgctgggtgggtcactagggagagacctgggcttgagaggtagagtgtggtgttgggggagtcaggtggcttgcggccattagagtcgcaggaccacactccccaggacagggcaggggccagcggtccagtggctggaggtggcccgtgatgaaggctacaaacctacccagccgcagccctgggaaggaagtgggctctacagggcagggcaccttttaccctggagctgcctgcttttgagggtaacagtcacgcccagccaagaccaggcctggggcgttagtgggtgacctaggcactgcggggcgggggggctgggtctacacagcctgggtctgggcccaccgtccgttgtatgtctgctatgcgcagccacagctgaactgccctcccagaccatctggaggccgctgggggactctggggaccaagactccatgtgccacagaggattgggggcggggcggtgctaggaactcaaagccagcctgggaagaccctgtccttgtcaccctttcttgccttgggtctgtccactgagtagcacacaagaccgggtgggcagggtccgttctgctccgggaatcacagactgtgtgtacccaggtggtgggcatgcagcgatcagtggcgtgggaccacagagggggcccgcggtacctaaaacagcttcacatggcttaaaataggggaccaatgtcttttccaatctaagtcccatttataataaagtccatgttccatttttaaaggacaatcctttcggtttaaaaccaggcacgattacccaaacaactcacaacggtaaagcactgtgaatcttctctgttctgcaatcccaacttggtttctgctcagaaaccctccctctttccaatcggtaattaaataacaaaaggaaaaaacttaagatgcttcaaccccgtttcgtgacactttgaaaaaagaatcacctcttgcaaacacccgctcccgacccccgccgctgaagcccggcgtccagaggcctaagcgcgggtgcccgcccccacccgggagcgcgggcctcgtggtcagcgcatccgcggggagaaacaaaggccgcggcacgggggctcaagggcactgcgccacaccgcacgcgcctacccccgcgcggccacgttaactggcggtcgccgcagcctcgggacagccggccgcgcgccgccaggctcgcggacgcgggaccacgcgccgccctccgggaggcccaagtctcgacccagccccgcgtggcgctgggggagggggcgcctccgccggaacgcgggtgggggaggggagggggaaatgcgctttgtctcgaaatggggcaaccgtcgccacagctccctaccccctcgagggcagagcagtccccccactaactaccgggctggccgcgcgccaggccagccgcgaggccaccgcccgaccctccactccttcccgcagctcccggcgcggggtccggcgagaaggggaggggaggggagcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcctcggcgagcgtttcggcgggcagcaggtcctcgtgagcgaggctgcggagcttcccctccccctctctcccgggaaccgatttggcggccgccattttcatggctcgccttcctctcagcgttttccttataactcttttattttcttagtgtgctttctctatcaagaagtagaagtggttaactattttttttttcttctcgggctgttttcatatcgtttcgaggtggatttggagtgttttgtgagcttggatctttagagtcctgcgcacctcattaaaggcgctcagccttcccctcgatgaaatggcgccattgcgttcggaagccacaccgaagagcggggagggggggtgctccgggtttgcgggcccggtttcagagaagatatcaccacccagggcgtcgggccgggttcaatgcgagccgtaggacaaagaaaccattttatgtttttcctgtcttttttttcctttgagtaacggttttatctgggtctgcagtcagtaaaacgacagatgaaccgcggcaaaataaacataaattggaagccatcggccacgaggggcagggacgaaggtggttttctgggcgggggagggatattcgcgtcagaatcctttactgttcttaaggattccgtttaagttgtagagctgactcattttaagtaatgttgttactgagaagtttaacccttacgggacagatccatggacctttatagatgattacgaggaaagtgaaataacgattttgtccttagttatacttcgattaaaacatggcttcagaggctccttcctgtaatgcgtatggattgatgtgcaaaactgttttgggcctgggccgctctgtatttgaactttgttacttttctcattttgtttgcaatcttggttgaacattacattgataagcataaggtctcaagcgaagggggtctacctggttatttttctttgaccctaagcacgtttataaaataacattgtttaaaatcgatagtggacatcgggtaagtttggataaattgtgaggtaagtaatgagtttttgctttttgttagtgatttgtaaaacttgttataaatgtacattatccgtaatttcagtttagagataacctatgtgctgacgacaattaagaataaaaactagctgaaaaaatgaaaataactatcgtgacaagtaaccatttcaaaagactgctttgtgtctcataggagctagtttgatcatttcagttaattttttctttaatttttacgagtcatgaaaactacaggaaaaaaaatctgaactgggttttaccactactttttaggagttgggagcatgcgaatggagggagagctccgtagaactgggatgagagcagcaattaatgctgcttgctaggaacaaaaaataattgattgaaaattacgtgtgactttttagtttgcattatgcgtttgtagcagttggtcctggatatcactttctctcgtttgaggttttttaacctagttaacttttaagacaggtttccttaacattcataagtgcccagaatacagctgtgtagtacagcatataaagatttcagctctgaggtttttcctattgacttggaaaattgttttgtgcctgtcgcttgccacatggccaatcaagtaagcttcgaattcgagctcgcccaactccgcccgttttatgactagaaccaatagtttttaatgccaaatgcactgaaatcccctaatttgcaaagccaaacgccccctatgtgagtaatacggggactttttacccaatttcccaagcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcagcccgggtctagaggatccgcctgagaaaggaagtgagctgtaaaggctgagctctctctctgacgtatgtagcctctggttagcttcgtcactcactgttcttgactcagcatggcaatctgatgaaatcccagctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctccagatccactagttctagagcaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaccagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagaacagatggaaggcctcctggcgccgcaccggcccaaggagcccgcgtggttcctggccaccgtcgcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcataggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgagatctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagggggtacctaggccgggcaacaattggcggccggccgcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccctaggccgggcaacaattggcggccggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactc

SEQ ID NO: 28 - DNA, 11998 bp, Artificial Sequence
  Artificial Sequence containing human UCOE elements and vector sequence
acgttgtaaaacgacggccagtgaattgtaatacgactcactatagggcgaattgggtaccgggccccccctcgaggtcgagttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtcgacggtatcaaggtggcgaccggaatggtgagctgcgagaatagccgggcgcgctgtgagccgaagtcgcccccgccctggccacttccggcgcgccgagtccttaggccgccagggggcgccggcgcgcgcccagattggggacaaaggaagccgggccggccgcgttattaccataaaaggcaaacactggtcggaggcgtccccgcggcgcgcggcaggaagccaggccccaaccccctcccaaccgggcgccagccccgcctccgcccggttcaaacagcgaccgggtcgcgcgcgcgcacgcagcggccacaccctcgggcgccagcggctcgggcaggaagtggcgcaagcgcccgggccccagaacgcacgcgcgattagcgccattgagtcccagcgcgcacgcgcaattagcgccaattcccagcgcgcacgcagttagcgcccaaaggaccagcgcgcacgcgcatggcgccccagcccccaccgggcctgacgggggctacgccgcgcccaccgtgcgatccccattggcaagagcccggctcagacaaagaccccgccggttgcccccgccccgagagcggcacccccggagcgcgcccgcccgagcgcggcctcgcgcctgcgaactggcgtggggtgtcccccatctccggaggcccaggggcttctcccgcgccccccacggcggtccggttccgccccatgcgccccccgctgcggcccagacggcggctctgcacgggcgaagggccgcggccgcatgccccggtcggctggccgggcttacctggcggcgggtgtggacgggcggcggatcggcaaaggcgaggctctgtgctcgcgggcggacgcggtctcggcggtggtggcgcgtcgcgccgctgggttttatagggcgccgccgcggccgctcgagccataaaaggcaactttcggaacggcgcacgctgattggccccgcgccgctcactcaccggcttcgccgcacagtgcagcatttttttaccccctctcccctccttttgcgaaaaaaaaaaagagcgagagcgagattgaggaagaggaggagggagagttttggcgttggccgccttggggtgctgggcccgggggctgggggcgcgcgccgtggcccccgcgccccacgctgggcagtgcccggttcggccccgcatggccaggcctgcccccggcctgcccgtctctcgggccccccacccaccgcgggacatcctaggtgtggacatctcttgggcactgagcgcccaggtggggtgggccagggtctgcacgggtgccagggccctgggttctgtacgctcctgcagaaggagctcttggagggcatggagtggccaggcagtcactcccccttgccgacttcagagcaactgccctgaaagcagggcctgaggacctctggctgtggggctcagctagctaaatgtgctgggtgggtcactagggagagacctgggcttgagaggtagagtgtggtgttgggggagtcaggtggcttgcggccattagagtcgcaggaccacactccccaggacagggcaggggccagcggtccagtggctggaggtggcccgtgatgaaggctacaaacctacccagccgcagccctgggaaggaagtgggctctacagggcagggcaccttttaccctggagctgcctgcttttgagggtaacagtcacgcccagccaagaccaggcctggggcgttagtgggtgacctaggcactgcggggcgggggggctgggtctacacagcctgggtctgggcccaccgtccgttgtatgtctgctatgcgcagccacagctgaactgccctcccagaccatctggaggccgctgggggactctggggaccaagactccatgtgccacagaggattgggggcggggcggtgctaggaactcaaagccagcctgggaagaccctgtccttgtcaccctttcttgccttgggtctgtccactgagtagcacacaagaccgggtgggcagggtccgttctgctccgggaatcacagactgtgtgtacccaggtggtgggcatgcagcgatcagtggcgtgggaccacagagggggcccgcggtacctaaaacagcttcacatggcttaaaataggggaccaatgtcttttccaatctaagtcccatttataataaagtccatgttccatttttaaaggacaatcctttcggtttaaaaccaggcacgattacccaaacaactcacaacggtaaagcactgtgaatcttctctgttctgcaatcccaacttggtttctgctcagaaaccctccctctttccaatcggtaattaaataacaaaaggaaaaaacttaagatgcttcaaccccgtttcgtgacactttgaaaaaagaatcacctcttgcaaacacccgctcccgacccccgccgctgaagcccggcgtccagaggcctaagcgcgggtgcccgcccccacccgggagcgcgggcctcgtggtcagcgcatccgcggggagaaacaaaggccgcggcacgggggctcaagggcactgcgccacaccgcacgcgcctacccccgcgcggccacgttaactggcggtcgccgcagcctcgggacagccggccgcgcgccgccaggctcgcggacgcgggaccacgcgccgccctccgggaggcccaagtctcgacccagccccgcgtggcgctgggggagggggcgcctccgccggaacgcgggtgggggaggggagggggaaatgcgctttgtctcgaaatggggcaaccgtcgccacagctccctaccccctcgagggcagagcagtccccccactaactaccgggctggccgcgcgccaggccagccgcgaggccaccgcccgaccctccactccttcccgcagctcccggcgcggggtccggcgagaaggggaggggaggggagcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcctcggcgagcgtttcggcgggcagcaggtcctcgtgagcgaggctgcggagcttcccctccccctctctcccgggaaccgatttggcggccgccattttcatggctcgccttcctctcagcgttttccttataactcttttattttcttagtgtgctttctctatcaagaagtagaagtggttaactattttttttttcttctcgggctgttttcatatcgtttcgaggtggatttggagtgttttgtgagcttggatctttagagtcctgcgcacctcattaaaggcgctcagccttcccctcgatgaaatggcgccattgcgttcggaagccacaccgaagagcggggagggggggtgctccgggtttgcgggcccggtttcagagaagatatcaccacccagggcgtcgggccgggttcaatgcgagccgtaggacaaagaaaccattttatgtttttcctgtcttttttttcctttgagtaacggttttatctgggtctgcagtcagtaaaacgacagatgaaccgcggcaaaataaacataaattggaagccatcggccacgaggggcagggacgaaggtggttttctgggcgggggagggatattcgcgtcagaatcctttactgttcttaaggattccgtttaagttgtagagctgactcattttaagtaatgttgttactgagaagtttaacccttacgggacagatccatggacctttatagatgattacgaggaaagtgaaataacgattttgtccttagttatacttcgattaaaacatggcttcagaggctccttcctgtaatgcgtatggattgatgtgcaaaactgttttgggcctgggccgctctgtatttgaactttgttacttttctcattttgtttgcaatcttggttgaacattacattgataagcataaggtctcaagcgaagggggtctacctggttatttttctttgaccctaagcacgtttataaaataacattgtttaaaatcgatagtggacatcgggtaagtttggataaattgtgaggtaagtaatgagtttttgctttttgttagtgatttgtaaaacttgttataaatgtacattatccgtaatttcagtttagagataacctatgtgctgacgacaattaagaataaaaactagctgaaaaaatgaaaataactatcgtgacaagtaaccatttcaaaagactgctttgtgtctcataggagctagtttgatcatttcagttaattttttctttaatttttacgagtcatgaaaactacaggaaaaaaaatctgaactgggttttaccactactttttaggagttgggagcatgcgaatggagggagagctccgtagaactgggatgagagcagcaattaatgctgcttgctaggaacaaaaaataattgattgaaaattacgtgtgactttttagtttgcattatgcgtttgtagcagttggtcctggatatcactttctctcgtttgaggttttttaacctagttaacttttaagacaggtttccttaacattcataagtgcccagaatacagctgtgtagtacagcatataaagatttcagctctgaggtttttcctattgacttggaaaattgttttgtgcctgtcgcttgccacatggccaatcaagtaagcttcgaattcgagctcgcccaactccgcccgttttatgactagaaccaatagtttttaatgccaaatgcactgaaatcccctaatttgcaaagccaaacgccccctatgtgagtaatacggggactttttacccaatttcccaagcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcagcccgggtctagaggatccgcctgagaaaggaagtgagctgtaaaggctgagctctctctctgacgtatgtagcctctggttagcttcgtcactcactgttcttgactcagcatggcaatctgatgaaatcccagctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctccagatccactagttctagagcaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaccagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcataggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgagatctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagggggtacctaggccgggcaacaattggcggccggccgcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccctaggccgggcaacaattggcggccggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactc

SEQ ID NO: 29 - DNA, 12052 bp, Artificial Sequence
  Artificial Sequence containing human UCOE elements and vector sequence
acgttgtaaaacgacggccagtgaattgtaatacgactcactatagggcgaattgggtaccgggccccccctcgaggtcgagttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtcgacggtatcaaggtggcgaccggaatggtgagctgcgagaatagccgggcgcgctgtgagccgaagtcgcccccgccctggccacttccggcgcgccgagtccttaggccgccagggggcgccggcgcgcgcccagattggggacaaaggaagccgggccggccgcgttattaccataaaaggcaaacactggtcggaggcgtccccgcggcgcgcggcaggaagccaggccccaaccccctcccaaccgggcgccagccccgcctccgcccggttcaaacagcgaccgggtcgcgcgcgcgcacgcagcggccacaccctcgggcgccagcggctcgggcaggaagtggcgcaagcgcccgggccccagaacgcacgcgcgattagcgccattgagtcccagcgcgcacgcgcaattagcgccaattcccagcgcgcacgcagttagcgcccaaaggaccagcgcgcacgcgcatggcgccccagcccccaccgggcctgacgggggctacgccgcgcccaccgtgcgatccccattggcaagagcccggctcagacaaagaccccgccggttgcccccgccccgagagcggcacccccggagcgcgcccgcccgagcgcggcctcgcgcctgcgaactggcgtggggtgtcccccatctccggaggcccaggggcttctcccgcgccccccacggcggtccggttccgccccatgcgccccccgctgcggcccagacggcggctctgcacgggcgaagggccgcggccgcatgccccggtcggctggccgggcttacctggcggcgggtgtggacgggcggcggatcggcaaaggcgaggctctgtgctcgcgggcggacgcggtctcggcggtggtggcgcgtcgcgccgctgggttttatagggcgccgccgcggccgctcgagccataaaaggcaactttcggaacggcgcacgctgattggccccgcgccgctcactcaccggcttcgccgcacagtgcagcatttttttaccccctctcccctccttttgcgaaaaaaaaaaagagcgagagcgagattgaggaagaggaggagggagagttttggcgttggccgccttggggtgctgggcccgggggctgggggcgcgcgccgtggcccccgcgccccacgctgggcagtgcccggttcggccccgcatggccaggcctgcccccggcctgcccgtctctcgggccccccacccaccgcgggacatcctaggtgtggacatctcttgggcactgagcgcccaggtggggtgggccagggtctgcacgggtgccagggccctgggttctgtacgctcctgcagaaggagctcttggagggcatggagtggccaggcagtcactcccccttgccgacttcagagcaactgccctgaaagcagggcctgaggacctctggctgtggggctcagctagctaaatgtgctgggtgggtcactagggagagacctgggcttgagaggtagagtgtggtgttgggggagtcaggtggcttgcggccattagagtcgcaggaccacactccccaggacagggcaggggccagcggtccagtggctggaggtggcccgtgatgaaggctacaaacctacccagccgcagccctgggaaggaagtgggctctacagggcagggcaccttttaccctggagctgcctgcttttgagggtaacagtcacgcccagccaagaccaggcctggggcgttagtgggtgacctaggcactgcggggcgggggggctgggtctacacagcctgggtctgggcccaccgtccgttgtatgtctgctatgcgcagccacagctgaactgccctcccagaccatctggaggccgctgggggactctggggaccaagactccatgtgccacagaggattgggggcggggcggtgctaggaactcaaagccagcctgggaagaccctgtccttgtcaccctttcttgccttgggtctgtccactgagtagcacacaagaccgggtgggcagggtccgttctgctccgggaatcacagactgtgtgtacccaggtggtgggcatgcagcgatcagtggcgtgggaccacagagggggcccgcggtaccaagcttgggaattgcgtgcaaaaacaacttctgttttccagggtaaacagaatctaatgcagaatctaatgcagggtaaacagacttaatgcagaatctaatgatggcacaaattaaaaatcactaacgtgccctttttagtgtgaaacccagagagagcacatacaagccaaaaacaaatgctttattttacctaggagacattaacattcacctttacgtgtttaagattaatgcaatgttaaatattgtgaaaactgtaactttgaatttcatgatttttatgtgaatattccagggtttaaaaaaacttgtaacatgacatggctgaataagataaaaaaaaaatctagccttttctcccttctggctcatatttgcgatttcgatcattttgtttaaaaaacaaaacactgcaatgaattaaacttaatattcttctatgttttagagtaagttaaaacaagataaagtgaccaaagtaatttgaaagattcaatgacttttgctccaacctaggtgcacaaggtaccttgttctttaaattgggctttaatgaaaatacttctccagaattctggggatttaagaaaaattatgccaaccaacaagggctttaccattttatgtaacatttttcaacgctgcaaaaatgtgtgtatttctatttgaagataaaaatcctcagcaaaatccacattgcactgtccttcaaagattagccttctttgaactagttaagacactattaagccaagccagtatctccctgtaatgaattcgtttttctcttaattttcccctgtaatttacactgggagagctgggaaatatgtggatgtaaatttctcagccacagagatgcaaagttatactgtggggaaaaaaaacttgagttaaatccttacatattttaggttttcattaacttaccaatgtagttttgttggaggccattttttttattgcagacttgaagagctattactagaaaaatgcatgacagttaaggtaagtttgcatgacacaaaaaaggtaactaaatacaaattctgtttggattccaacccccaagtagagagcgcacactttcaaacgtgaatacaaatccagagtagatctgcgctcctacctacattgcttatgatgtacttaagtacgtgtcctaaccatgtgagtctagaaagactttactggggatcctggtacctaaaacagcttcacatggcttaaaataggggaccaatgtcttttccaatctaagtcccatttataataaagtccatgttccatttttaaaggacaatcctttcggtttaaaaccaggcacgattacccaaacaactcacaacggtaaagcactgtgaatcttctctgttctgcaatcccaacttggtttctgctcagaaaccctccctctttccaatcggtaattaaataacaaaaggaaaaaacttaagatgcttcaaccccgtttcgtgacactttgaaaaaagaatcacctcttgcaaacacccgctcccgacccccgccgctgaagcccggcgtccagaggcctaagcgcgggtgcccgcccccacccgggagcgcgggcctcgtggtcagcgcatccgcggggagaaacaaaggccgcggcacgggggctcaagggcactgcgccacaccgcacgcgcctacccccgcgcggccacgttaactggcggtcgccgcagcctcgggacagccggccgcgcgccgccaggctcgcggacgcgggaccacgcgccgccctccgggaggcccaagtctcgacccagccccgcgtggcgctgggggagggggcgcctccgccggaacgcgggtgggggaggggagggggaaatgcgctttgtctcgaaatggggcaaccgtcgccacagctccctaccccctcgagggcagagcagtccccccactaactaccgggctggccgcgcgccaggccagccgcgaggccaccgcccgaccctccactccttcccgcagctcccggcgcggggtccggcgagaaggggaggggaggggagcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcctcggcgagcgtttcggcgggcagcaggtcctcgtgagcgaggctgcggagcttcccctccccctctctcccgggaaccgatttggcggccgccattttcatggctcgccttcctctcagcgttttccttataactcttttattttcttagtgtgctttctctatcaagaagtagaagtggttaactattttttttttcttctcgggctgttttcatatcgtttcgaggtggatttggagtgttttgtgagcttggatctttagagtcctgcgcacctcattaaaggcgctcagccttcccctcgatgaaatggcgccattgcgttcggaagccacaccgaagagcggggagggggggtgctccgggtttgcgggcccggtttcagagaagatcccaagcttcgaattcgagctcgcccaactccgcccgttttatgactagaaccaatagtttttaatgccaaatgcactgaaatcccctaatttgcaaagccaaacgccccctatgtgagtaatacggggactttttacccaatttcccaagcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcagcccgggtctagaggatccgcctgagaaaggaagtgagctgtaaaggctgagctctctctctgacgtatgtagcctctggttagcttcgtcactcactgttcttgactcagcatggcaatctgatgaaatcccagctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctccagatccactagtaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcstggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgagactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagggggtacctaggccgggcaacaattggcggccggccgcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccctaggccgggcaacaattggcggccggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactc

SEQ ID NO: 30 - DNA, 11941 bp, Artificial Sequence
  Artificial Sequence containing human UCOE elements and vector sequence
acgttgtaaaacgacggccagtgaattgtaatacgactcactatagggcgaattgggtaccgggccccccctcgaggtcgagttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtcgacggtatcaaggtggcgaccggaatggtgagctgcgagaatagccgggcgcgctgtgagccgaagtcgcccccgccctggccacttccggcgcgccgagtccttaggccgccagggggcgccggcgcgcgcccagattggggacaaaggaagccgggccggccgcgttattaccataaaaggcaaacactggtcggaggcgtccccgcggcgcgcggcaggaagccaggccccaaccccctcccaaccgggcgccagccccgcctccgcccggttcaaacagcgaccgggtcgcgcgcgcgcacgcagcggccacaccctcgggcgccagcggctcgggcaggaagtggcgcaagcgcccgggccccagaacgcacgcgcgattagcgccattgagtcccagcgcgcacgcgcaattagcgccaattcccagcgcgcacgcagttagcgcccaaaggaccagcgcgcacgcgcatggcgccccagcccccaccgggcctgacgggggctacgccgcgcccaccgtgcgatccccattggcaagagcccggctcagacaaagaccccgccggttgcccccgccccgagagcggcacccccggagcgcgcccgcccgagcgcggcctcgcgcctgcgaactggcgtggggtgtcccccatctccggaggcccaggggcttctcccgcgccccccacggcggtccggttccgccccatgcgccccccgctgcggcccagacggcggctctgcacgggcgaagggccgcggccgcatgccccggtcggctggccgggcttacctggcggcgggtgtggacgggcggcggatcggcaaaggcgaggctctgtgctcgcgggcggacgcggtctcggcggtggtggcgcgtcgcgccgctgggttttatagggcgccgccgcggccgctcgagccataaaaggcaactttcggaacggcgcacgctgattggccccgcgccgctcactcaccggcttcgccgcacagtgcagcatttttttaccccctctcccctccttttgcgaaaaaaaaaaagagcgagagcgagattgaggaagaggaggagggagagttttggcgttggccgccttggggtgctgggcccgggggctgggggcgcgcgccgtggcccccgcgccccacgctgggcagtgcccggttcggccccgcatggccaggcctgcccccggcctgcccgtctctcgggccccccacccaccgcgggacatcctaggtgtggacatctcttgggcactgagcgcccaggtggggtgggccagggtctgcacgggtgccagggccctgggttctgtacgctcctgcagaaggagctcttggagggcatggagtggccaggcagtcactcccccttgccgacttcagagcaactgccctgaaagcagggcctgaggacctctggctgtggggctcagctagctaaatgtgctgggtgggtcactagggagagacctgggcttgagaggtagagtgtggtgttgggggagtcaggtggcttgcggccattagagtcgcaggaccacactccccaggacagggcaggggccagcggtccagtggctggaggtggcccgtgatgaaggctacaaacctacccagccgcagccctgggaaggaagtgggctctacagggcagggcaccttttaccctggagctgcctgcttttgagggtaacagtcacgcccagccaagaccaggcctggggcgttagtgggtgacctaggcactgcggggcgggggggctgggtctacacagcctgggtctgggcccaccgtccgttgtatgtctgctatgcgcagccacagctgaactgccctcccagaccatctggaggccgctgggggactctggggaccaagactccatgtgccacagaggattgggggcggggcggtgctaggaactcaaagccagcctgggaagaccctgtccttgtcaccctttcttgccttgggtctgtccactgagtagcacacaagaccgggtgggcagggtccgttctgctccgggaatcacagactgtgtgtacccaggtggtgggcatgcagcgatcagtggcgtgggaccacagagggggcccgcggtaccaagcttgggaattgcgtgcaaaaacaacttctgttttccagggtaaacagaatctaatgcagaatctaatgcagggtaaacagacttaatgcagaatctaatgatggcacaaattaaaaatcactaacgtgccctttttagtgtgaaacccagagagagcacatacaagccaaaaacaaatgctttattttacctaggagacattaacattcacctttacgtgtttaagattaatgcaatgttaaatattgtgaaaactgtaactttgaatttcatgatttttatgtgaatattccagggtttaaaaaaacttgtaacatgacatggctgaataagataaaaaaaaaatctagccttttctcccttctggctcatatttgcgatttcgatcattttgtttaaaaaacaaaacactgcaatgaattaaacttaatattcttctatgttttagagtaagttaaaacaagataaagtgaccaaagtaatttgaaagattcaatgacttttgctccaacctaggtgcacaaggtaccttgttctttaaattgggctttaatgaaaatacttctccagaattctggggatttaagaaaaattatgccaaccaacaagggctttaccattttatgtaacatttttcaacgctgcaaaaatgtgtgtatttctatttgaagataaaaatcctcagcaaaatccacattgcactgtccttcaaagattagccttctttgaactagttaagacactattaagccaagccagtatctccctgtaatgaattcgtttttctcttaattttcccctgtaatttacactgggagagctgggaaatatgtggatgtaaatttctcagccacagagatgcaaagttatactgtggggaaaaaaaacttgagttaaatccttacatattttaggttttcattaacttaccaatgtagttttgttggaggccattttttttattgcagacttgaagagctattactagaaaaatgcatgacagttaaggtaagtttgcatgacacaaaaaaggtaactaaatacaaattctgtttggattccaacccccaagtagagagcgcacactttcaaacgtgaatacaaatccagagtagatctgcgctcctacctacattgcttatgatgtacttaagtacgtgtcctaaccatgtgagtctagaaagactttactggggatcctggtacctaaaacagcttcacatggcttaaaataggggaccaatgtcttttccaatctaagtcccatttataataaagtccatgttccatttttaaaggacaatcctttcggtttaaaaccaggcacgattacccaaacaactcacaacggtaaagcactgtgaatcttctctgttctgcaatcccaacttggtttctgctcagaaaccctccctctttccaatcggtaattaaataacaaaaggaaaaaacttaagatgcttcaaccccgtttcgtgacactttgaaaaaagaatcacctcttgcaaacacccgctcccgacccccgccgctgaagcccggcgtccagaggcctaagcgcgggtgcccgcccccacccgggagcgcgggcctcgtggtcagcgcatccgcggggagaaacaaaggccgcggcacgggggctcaagggcactgcgccacaccgcacgcgcctacccccgcgcggccacgttaactggcggtcgccgcagcctcgggacagccggccgcgcgccgccaggctcgcggacgcgggaccacgcgccgccctccgggaggcccaagtctcgacccagccccgcgtggcgctgggggagggggcgcctccgccggaacgcgggtgggggaggggagggggaaatgcgctttgtctcgaaatggggcaaccgtcgccacagctccctaccccctcgagggcagagcagtccccccactaactaccgggctggccgcgcgccaggccagccgcgaggccaccgcccgaccctccactccttcccgcagctcccggcgcggggtccggcgagaaggggaggggaggggagcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcctcggcgagcgtttcggcgggcagcaggtcctcgtgagcgaggctgcggagcttcccctccccctctctcccgggaaccgatttggcggccgccattttcatggctcgccttcctctcagcgttttccttataactcttttattttcttagtgtgctttctctatcaagaagtagaagtggttaactattttttttttcttctcgggctgttttcatatcgtttcgaggtggatttggagtgttttgtgagcttggatctttagagtcctgcgcacctcattaaaggcgctcagccttcccctcgatgaaatggcgccattgcgttcggaagccacaccgaagagcggggagggggggtgctccgggtttgcgggcccggtttcagagaagatcccaagcttcgaattcgagctcgcccaactccgcccgttttatgactagaaccaatagtttttaatgccaaatgcactgaaatcccctaatttgcaaagccaaacgccccctatgtgagtaatacggggactttttacccaatttcccaagcggaaagccccctaatacactcatatggcatatgaatcagcacggtcatgcactctaatggcggcccatagggactttccacatagggggcgttcaccatttcccagcataggggtggtgactcaatggcctttacccaagtacattgggtcaatgggaggtaagccaatgggtttttcccattactggcaagcacactgagtcaaatgggactttccactgggttttgcccaagtacattgggtcaatgggaggtgagccaatgggaaaaacccattgctgccaagtacactgactcaatagggactttccaatgggtttttccattgttggcaagcatataaggtcaatgtgggtgagtcaatagggactttccattgtattctgcccagtacataaggtcaatagggggtgaatcaacaggaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagtcccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttcccctggaaattccatattggcacgcattctattggctgagctgcgttctacgtgggtataagaggcgcgaccagcgtcggtaccgtcgcagtcttcggtctgaccaccgtagaacgcagagctcctcgctgcagcccgggtctagaggatccgcctgagaaaggaagtgagctgtaaaggctgagctctctctctgacgtatgtagcctctggttagcttcgtcactcactgttcttgactcagcatggcaatctgatgaaatcccagctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctccagatccactagttctagagcaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaccagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcataggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgagatctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagggggtacctaggccgggcaacaattggcggccggccgcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccctaggccgggcaacaattggcggccggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactc

SEQ ID NO: 31 - DNA, 11216 bp, Artificial Sequence
  Artificial Sequence containing human UCOE elements and vector sequence
acgttgtaaaacgacggccagtgaattgtaatacgactcactatagggcgaattgggtaccgggccccccctcgaggtcgagttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtcgacggtatcaaggtggcgaccggaatggtgagctgcgagaatagccgggcgcgctgtgagccgaagtcgcccccgccctggccacttccggcgcgccgagtccttaggccgccagggggcgccggcgcgcgcccagattggggacaaaggaagccgggccggccgcgttattaccataaaaggcaaacactggtcggaggcgtccccgcggcgcgcggcaggaagccaggccccaaccccctcccaaccgggcgccagccccgcctccgcccggttcaaacagcgaccgggtcgcgcgcgcgcacgcagcggccacaccctcgggcgccagcggctcgggcaggaagtggcgcaagcgcccgggccccagaacgcacgcgcgattagcgccattgagtcccagcgcgcacgcgcaattagcgccaattcccagcgcgcacgcagttagcgcccaaaggaccagcgcgcacgcgcatggcgccccagcccccaccgggcctgacgggggctacgccgcgcccaccgtgcgatccccattggcaagagcccggctcagacaaagaccccgccggttgcccccgccccgagagcggcacccccggagcgcgcccgcccgagcgcggcctcgcgcctgcgaactggcgtggggtgtcccccatctccggaggcccaggggcttctcccgcgccccccacggcggtccggttccgccccatgcgccccccgctgcggcccagacggcggctctgcacgggcgaagggccgcggccgcatgccccggtcggctggccgggcttacctggcggcgggtgtggacgggcggcggatcggcaaaggcgaggctctgtgctcgcgggcggacgcggtctcggcggtggtggcgcgtcgcgccgctgggttttatagggcgccgccgcggccgctcgagccataaaaggcaactttcggaacggcgcacgctgattggccccgcgccgctcactcaccggcttcgccgcacagtgcagcatttttttaccccctctcccctccttttgcgaaaaaaaaaaagagcgagagcgagattgaggaagaggaggagggagagttttggcgttggccgccttggggtgctgggcccgggggctgggggcgcgcgccgtggcccccgcgccccacgctgggcagtgcccggttcggccccgcatggccaggcctgcccccggcctgcccgtctctcgggccccccacccaccgcgggacatcctaggtgtggacatctcttgggcactgagcgcccaggtggggtgggccagggtctgcacgggtgccagggccctgggttctgtacgctcctgcagaaggagctcttggagggcatggagtggccaggcagtcactcccccttgccgacttcagagcaactgccctgaaagcagggcctgaggacctctggctgtggggctcagctagctaaatgtgctgggtgggtcactagggagagacctgggcttgagaggtagagtgtggtgttgggggagtcaggtggcttgcggccattagagtcgcaggaccacactccccaggacagggcaggggccagcggtccagtggctggaggtggcccgtgatgaaggctacaaacctacccagccgcagccctgggaaggaagtgggctctacagggcagggcaccttttaccctggagctgcctgcttttgagggtaacagtcacgcccagccaagaccaggcctggggcgttagtgggtgacctaggcactgcggggcgggggggctgggtctacacagcctgggtctgggcccaccgtccgttgtatgtctgctatgcgcagccacagctgaactgccctcccagaccatctggaggccgctgggggactctggggaccaagactccatgtgccacagaggattgggggcggggcggtgctaggaactcaaagccagcctgggaagaccctgtccttgtcaccctttcttgccttgggtctgtccactgagtagcacacaagaccgggtgggcagggtccgttctgctccgggaatcacagactgtgtgtacccaggtggtgggcatgcagcgatcagtggcgtgggaccacagagggggcccgcggtaccaagcttgggaattgcgtgcaaaaacaacttctgttttccagggtaaacagaatctaatgcagaatctaatgcagggtaaacagacttaatgcagaatctaatgatggcacaaattaaaaatcactaacgtgccctttttagtgtgaaacccagagagagcacatacaagccaaaaacaaatgctttattttacctaggagacattaacattcacctttacgtgtttaagattaatgcaatgttaaatattgtgaaaactgtaactttgaatttcatgatttttatgtgaatattccagggtttaaaaaaacttgtaacatgacatggctgaataagataaaaaaaaaatctagccttttctcccttctggctcatatttgcgatttcgatcattttgtttaaaaaacaaaacactgcaatgaattaaacttaatattcttctatgttttagagtaagttaaaacaagataaagtgaccaaagtaatttgaaagattcaatgacttttgctccaacctaggtgcacaaggtaccttgttctttaaattgggctttaatgaaaatacttctccagaattctggggatttaagaaaaattatgccaaccaacaagggctttaccattttatgtaacatttttcaacgctgcaaaaatgtgtgtatttctatttgaagataaaaatcctcagcaaaatccacattgcactgtccttcaaagattagccttctttgaactagttaagacactattaagccaagccagtatctccctgtaatgaattcgtttttctcttaattttcccctgtaatttacactgggagagctgggaaatatgtggatgtaaatttctcagccacagagatgcaaagttatactgtggggaaaaaaaacttgagttaaatccttacatattttaggttttcattaacttaccaatgtagttttgttggaggccattttttttattgcagacttgaagagctattactagaaaaatgcatgacagttaaggtaagtttgcatgacacaaaaaaggtaactaaatacaaattctgtttggattccaacccccaagtagagagcgcacactttcaaacgtgaatacaaatccagagtagatctgcgctcctacctacattgcttatgatgtacttaagtacgtgtcctaaccatgtgagtctagaaagactttactggggatcctggtacctaaaacagcttcacatggcttaaaataggggaccaatgtcttttccaatctaagtcccatttataataaagtccatgttccatttttaaaggacaatcctttcggtttaaaaccaggcacgattacccaaacaactcacaacggtaaagcactgtgaatcttctctgttctgcaatcccaacttggtttctgctcagaaaccctccctctttccaatcggtaattaaataacaaaaggaaaaaacttaagatgcttcaaccccgtttcgtgacactttgaaaaaagaatcacctcttgcaaacacccgctcccgacccccgccgctgaagcccggcgtccagaggcctaagcgcgggtgcccgcccccacccgggagcgcgggcctcgtggtcagcgcatccgcggggagaaacaaaggccgcggcacgggggctcaagggcactgcgccacaccgcacgcgcctacccccgcgcggccacgttaactggcggtcgccgcagcctcgggacagccggccgcgcgccgccaggctcgcggacgcgggaccacgcgccgccctccgggaggcccaagtctcgacccagccccgcgtggcgctgggggagggggcgcctccgccggaacgcgggtgggggaggggagggggaaatgcgctttgtctcgaaatggggcaaccgtcgccacagctccctaccccctcgagggcagagcagtccccccactaactaccgggctggccgcgcgccaggccagccgcgaggccaccgcccgaccctccactccttcccgcagctcccggcgcggggtccggcgagaaggggaggggaggggagcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcctcggcgagcgtttcggcgggcagcaggtcctcgtgagcgaggctgcggagcttcccctccccctctctcccgggaaccgatttggcggccgccattttcatggctcgccttcctctcagcgttttccttataactcttttattttcttagtgtgctttctctatcaagaagtagaagtggttaactattttttttttcttctcgggctgttttcatatcgtttcgaggtggatttggagtgttttgtgagcttggatctttagagtcctgcgcacctcattaaaggcgctcagccttcccctcgatgaaatggcgccattgcgttcggaagccacaccgaagagcggggagggggggtgctccgggtttgcgggcccggtttcagagaagatcccaagcttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcggatccgcctgagaaaggaagtgagctgtaaaggctgagctctctctctgacgtatgtagcctctggttagcttcgtcactcactgttcttgactcagcatggcaatctgatgaaatcccagctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctccagatccactagtaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcstggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgagactagttctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagggggtacctaggccgggcaacaattggcggccggccgcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccctaggccgggcaacaattggcggccggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactc

SEQ ID NO: 32 - DNA, 11105 bp, Artificial Sequence
  Artificial Sequence containing human UCOE elements and vector sequence
acgttgtaaaacgacggccagtgaattgtaatacgactcactatagggcgaattgggtaccgggccccccctcgaggtcgagttggggtggggaaaaggaagaaacgcgggcgtattggccccaatggggtctcggtggggtatcgacagagtgccagccctgggaccgaaccccgcgtttatgaacaaacgacccaacacccgtgcgttttattctgtctttttattgccgtcatagcgcgggttccttccggtattgtctccttccgtcgacggtatcaaggtggcgaccggaatggtgagctgcgagaatagccgggcgcgctgtgagccgaagtcgcccccgccctggccacttccggcgcgccgagtccttaggccgccagggggcgccggcgcgcgcccagattggggacaaaggaagccgggccggccgcgttattaccataaaaggcaaacactggtcggaggcgtccccgcggcgcgcggcaggaagccaggccccaaccccctcccaaccgggcgccagccccgcctccgcccggttcaaacagcgaccgggtcgcgcgcgcgcacgcagcggccacaccctcgggcgccagcggctcgggcaggaagtggcgcaagcgcccgggccccagaacgcacgcgcgattagcgccattgagtcccagcgcgcacgcgcaattagcgccaattcccagcgcgcacgcagttagcgcccaaaggaccagcgcgcacgcgcatggcgccccagcccccaccgggcctgacgggggctacgccgcgcccaccgtgcgatccccattggcaagagcccggctcagacaaagaccccgccggttgcccccgccccgagagcggcacccccggagcgcgcccgcccgagcgcggcctcgcgcctgcgaactggcgtggggtgtcccccatctccggaggcccaggggcttctcccgcgccccccacggcggtccggttccgccccatgcgccccccgctgcggcccagacggcggctctgcacgggcgaagggccgcggccgcatgccccggtcggctggccgggcttacctggcggcgggtgtggacgggcggcggatcggcaaaggcgaggctctgtgctcgcgggcggacgcggtctcggcggtggtggcgcgtcgcgccgctgggttttatagggcgccgccgcggccgctcgagccataaaaggcaactttcggaacggcgcacgctgattggccccgcgccgctcactcaccggcttcgccgcacagtgcagcatttttttaccccctctcccctccttttgcgaaaaaaaaaaagagcgagagcgagattgaggaagaggaggagggagagttttggcgttggccgccttggggtgctgggcccgggggctgggggcgcgcgccgtggcccccgcgccccacgctgggcagtgcccggttcggccccgcatggccaggcctgcccccggcctgcccgtctctcgggccccccacccaccgcgggacatcctaggtgtggacatctcttgggcactgagcgcccaggtggggtgggccagggtctgcacgggtgccagggccctgggttctgtacgctcctgcagaaggagctcttggagggcatggagtggccaggcagtcactcccccttgccgacttcagagcaactgccctgaaagcagggcctgaggacctctggctgtggggctcagctagctaaatgtgctgggtgggtcactagggagagacctgggcttgagaggtagagtgtggtgttgggggagtcaggtggcttgcggccattagagtcgcaggaccacactccccaggacagggcaggggccagcggtccagtggctggaggtggcccgtgatgaaggctacaaacctacccagccgcagccctgggaaggaagtgggctctacagggcagggcaccttttaccctggagctgcctgcttttgagggtaacagtcacgcccagccaagaccaggcctggggcgttagtgggtgacctaggcactgcggggcgggggggctgggtctacacagcctgggtctgggcccaccgtccgttgtatgtctgctatgcgcagccacagctgaactgccctcccagaccatctggaggccgctgggggactctggggaccaagactccatgtgccacagaggattgggggcggggcggtgctaggaactcaaagccagcctgggaagaccctgtccttgtcaccctttcttgccttgggtctgtccactgagtagcacacaagaccgggtgggcagggtccgttctgctccgggaatcacagactgtgtgtacccaggtggtgggcatgcagcgatcagtggcgtgggaccacagagggggcccgcggtaccaagcttgggaattgcgtgcaaaaacaacttctgttttccagggtaaacagaatctaatgcagaatctaatgcagggtaaacagacttaatgcagaatctaatgatggcacaaattaaaaatcactaacgtgccctttttagtgtgaaacccagagagagcacatacaagccaaaaacaaatgctttattttacctaggagacattaacattcacctttacgtgtttaagattaatgcaatgttaaatattgtgaaaactgtaactttgaatttcatgatttttatgtgaatattccagggtttaaaaaaacttgtaacatgacatggctgaataagataaaaaaaaaatctagccttttctcccttctggctcatatttgcgatttcgatcattttgtttaaaaaacaaaacactgcaatgaattaaacttaatattcttctatgttttagagtaagttaaaacaagataaagtgaccaaagtaatttgaaagattcaatgacttttgctccaacctaggtgcacaaggtaccttgttctttaaattgggctttaatgaaaatacttctccagaattctggggatttaagaaaaattatgccaaccaacaagggctttaccattttatgtaacatttttcaacgctgcaaaaatgtgtgtatttctatttgaagataaaaatcctcagcaaaatccacattgcactgtccttcaaagattagccttctttgaactagttaagacactattaagccaagccagtatctccctgtaatgaattcgtttttctcttaattttcccctgtaatttacactgggagagctgggaaatatgtggatgtaaatttctcagccacagagatgcaaagttatactgtggggaaaaaaaacttgagttaaatccttacatattttaggttttcattaacttaccaatgtagttttgttggaggccattttttttattgcagacttgaagagctattactagaaaaatgcatgacagttaaggtaagtttgcatgacacaaaaaaggtaactaaatacaaattctgtttggattccaacccccaagtagagagcgcacactttcaaacgtgaatacaaatccagagtagatctgcgctcctacctacattgcttatgatgtacttaagtacgtgtcctaaccatgtgagtctagaaagactttactggggatcctggtacctaaaacagcttcacatggcttaaaataggggaccaatgtcttttccaatctaagtcccatttataataaagtccatgttccatttttaaaggacaatcctttcggtttaaaaccaggcacgattacccaaacaactcacaacggtaaagcactgtgaatcttctctgttctgcaatcccaacttggtttctgctcagaaaccctccctctttccaatcggtaattaaataacaaaaggaaaaaacttaagatgcttcaaccccgtttcgtgacactttgaaaaaagaatcacctcttgcaaacacccgctcccgacccccgccgctgaagcccggcgtccagaggcctaagcgcgggtgcccgcccccacccgggagcgcgggcctcgtggtcagcgcatccgcggggagaaacaaaggccgcggcacgggggctcaagggcactgcgccacaccgcacgcgcctacccccgcgcggccacgttaactggcggtcgccgcagcctcgggacagccggccgcgcgccgccaggctcgcggacgcgggaccacgcgccgccctccgggaggcccaagtctcgacccagccccgcgtggcgctgggggagggggcgcctccgccggaacgcgggtgggggaggggagggggaaatgcgctttgtctcgaaatggggcaaccgtcgccacagctccctaccccctcgagggcagagcagtccccccactaactaccgggctggccgcgcgccaggccagccgcgaggccaccgcccgaccctccactccttcccgcagctcccggcgcggggtccggcgagaaggggaggggaggggagcggagaaccgggcccccgggacgcgtgtggcatctgaagcaccaccagcgagcgagagctagagagaaggaaagccaccgacttcaccgcctccgagctgctccgggtcgcgggtctgcagcgtctccggccctccgcgcctacagctcaagccacatccgaagggggagggagccgggagctgcgcgcggggccgccggggggaggggtggcaccgcccacgccgggcggccacgaagggcggggcagcgggcgcgcgcgcggcggggggaggggccggcgccgcgcccgctgggaattggggccctagggggagggcggaggcgccgacgaccgcggcacttaccgttcgcggcgtggcgcccggtggtccccaaggggagggaagggggaggcggggcgaggacagtgaccggagtctcctcagcggtggcttttctgcttggcagcctcagcggctggcgccaaaaccggactccgcccacttcctcgcccgccggtgcgagggtgtggaatcctccagacgctgggggagggggagttgggagcttaaaaactagtacccctttgggaccactttcagcagcgaactctcctgtacaccaggggtcagttccacagacgcgggccaggggtgggtcattgcggcgtgaacaataatttgactagaagttgattcgggtgtttccggaaggggccgagtcaatccgccgagttggggcacggaaaacaaaaagggaaggctactaagatttttctggcgggggttatcattggcgtaactgcagggaccacctcccgggttgagggggctggatctccaggctgcggattaagcccctcccgtcggcgttaatttcaaactgcgcgacgtttctcacctgccttcgccaaggcaggggccgggaccctattccaagaggtagtaactagcaggactctagccttccgcaattcattgagcgcatttacggaagtaacgtcgggtactgtctctggccgcaagggtgggaggagtacgcatttggcgtaaggtggggcgtagagccttcccgccattggcggcggatagggcgtttacgcgacggcctgacgtagcggaagacgcgttagtgggggggaaggttctagaaaagcggcggcagcggctctagcggcagtagcagcagcgccgggtcccgtgcggaggtgctcctcgcagagttgtttctcgagcagcggcagttctcactacagcgccaggacgagtccggttcgtgttcgtccgcggagatctctctcatctcgctcggctgcgggaaatcgggctgaagcgactgagtccgcgatggaggtaacgggtttgaaatcaatgagttattgaaaagggcatggcgaggccgttggcgcctcagtggaagtcggccagccgcctccgtgggagagaggcaggaaatcggaccaattcagtagcagtggggcttaaggtttatgaacggggtcttgagcggaggcctgagcgtacaaacagcttccccaccctcagcctcccggcgccatttcccttcactgggggtgggggatggggagctttcacatggcggacgctgccccgctggggtgaaagtggggcgcggaggcgggaattcttattccctttctaaagcacgctgcttcgggggccacggcgtctcctcggcgagcgtttcggcgggcagcaggtcctcgtgagcgaggctgcggagcttcccctccccctctctcccgggaaccgatttggcggccgccattttcatggctcgccttcctctcagcgttttccttataactcttttattttcttagtgtgctttctctatcaagaagtagaagtggttaactattttttttttcttctcgggctgttttcatatcgtttcgaggtggatttggagtgttttgtgagcttggatctttagagtcctgcgcacctcattaaaggcgctcagccttcccctcgatgaaatggcgccattgcgttcggaagccacaccgaagagcggggagggggggtgctccgggtttgcgggcccggtttcagagaagatcccaagcttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcggatccgcctgagaaaggaagtgagctgtaaaggctgagctctctctctgacgtatgtagcctctggttagcttcgtcactcactgttcttgactcagcatggcaatctgatgaaatcccagctgtaagtctgcagaaattgatgatctattaaacaataaagatgtccactaaaatggaagtttttcctgtcatactttgttaagaagggtgagaacagagtacctacattttgaatggaaggattggagctacgggggtgggggtggggtgggattagataaatgcctgctctttactgaaggctctttactattgctttatgataatgtttcatagttggatatcataatttaaacaagcaaaaccaaattaagggccagctcattcctccagatccactagttctagagcaaattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgaccagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcctgacgcccgccccacgacccgcagcgcccgaccgaaaggagcgcacgaccccatgcataggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgagatctagagcggccgccaccgcggtggagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagggggtacctaggccgggcaacaattggcggccggccgcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccctaggccgggcaacaattggcggccggccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactc